(19) United States
(12) Patent Application Publication
(10) Pub. No.: US 2021/0269825 A1
(43) Pub. Date: Sep. 2, 2021

(54) COMPOSITIONS AND METHODS FOR REDUCING SPLICEOPATHY AND TREATING RNA DOMINANCE DISORDERS

(71) Applicant: University of Washington, Seattle, WA (US)

(72) Inventor: Joel Chamberlain, Seattle, WA (US)

(73) Assignee: University of Washington, Seattle, WA (US)

(21) Appl. No.: 17/054,474

(22) PCT Filed: May 15, 2019
(86) PCT No.: PCT/US19/32423

§ 371 Date: Nov. 10, 2020

Related U.S. Application Data
(60) Provisional application No. 62/671,769, filed on May 15, 2018.

(51) Int. Cl. C12N 15/86; C12N 15/113; C12N 9/12; A61P 21/00

(52) U.S. Cl. CPC C12N 15/86; C12N 15/1137; C12N 9/12; C12Y 207/11001; A61P 21/00; C12N 2320/32; C12N 2310/14; C12N 2750/14171; C12N 2310/122; C12N 2310/531; C12N 2310/141; C12N 2750/14143

(57) ABSTRACT

The disclosure features compositions and methods for the treatment of disorders associated with improper ribonucleic acid (RNA) splicing, including disorders characterized by nuclear retention of RNA transcripts containing aberrantly expanded repeat regions that bind and sequester splicing factor proteins. Disclosed herein are interfering RNA constructs that suppress the expression of RNA transcripts containing expanded repeat regions, as well as viral vectors, such as adeno-associated viral vectors, encoding such interfering RNA molecules. For example, the disclosure features interfering RNA molecules, such as siRNA, miRNA, and shRNA constructs, that anneal to dystrophia myotonica protein kinase (DMPK) RNA transcripts and attenuate the expression of DMPK RNA containing expanded CUG trinucleotide repeats. Using the compositions and methods described herein, a patient having an RNA dominance disorder, such as a human patient having myotonic dystrophy, among other conditions described herein, may be administered an interfering RNA construct or vector containing the same so as to reduce the occurrence of spliceopathy in the patient, thereby treating an underlying etiology of the disease.

### GOVERNMENT LICENSE RIGHTS

This invention was made with government support under Grant No. R03 AR056107, awarded by the National Institutes of Health. The government has certain rights in the invention.

### FIELD OF THE INVENTION

The invention relates to the field of nucleic acid biotechnology and provides compositions and methods for treating genetic disorders associated with improper ribonucleic acid splicing.

### BACKGROUND OF THE INVENTION

The expression and nuclear retention of endogenous ribonucleic acid (RNA) transcripts containing aberrantly expanded repeat regions leads to the onset of RNA dominance, a pathology that underlies various heritable genetic disorders, including myotonic dystrophy type 1, among others. Myotonic dystrophy is the most common form of muscular dystrophy, and occurs with an estimate frequency of about 1 in 7,500 human adults. RNA dominance results from a gain-of-function mutation in RNA transcripts that imparts these molecules with undesired biological activity. In myotonic dystrophy, RNA dominance is effectuated by the presence of expanded CUG trinucleotide repeats in RNA transcript that encode dystrophia myotonica protein kinase (DMPK), which sequester RNA proteins that control RNA splicing, such as muscleblind-like protein, by virtue of the elevated avidity of these expansion regions for such splicing factor proteins. There is a paucity of strategies available for successfully treating and ameliorating the symptoms of myotonic dystrophy, among other disorders associated with RNA dominance, and there remains a need for effective therapeutics for these diseases.

### SUMMARY OF THE INVENTION

Described herein are compositions and methods useful for reducing the occurrence of spliceopathy and for treating disorders associated with ribonucleic acid (RNA) dominance, a pathology that is induced by the expression and nuclear retention of messenger RNA (mRNA) transcripts containing expanded repeat regions that bind and sequester splicing factor proteins, thereby interfering with the proper splicing of various mRNA transcripts. The compositions described herein that may be used to treat such disorders include nucleic acids containing interfering RNA constructs that suppress the expression of RNA transcripts containing aberrantly expanded repeat regions, such as siRNA, miRNA, and shRNA constructs that anneal to portions of nuclear-retained, repeat-expanded RNA transcripts and promote the degradation of these pathological transcripts by way of various cellular processes. The present disclosure additionally features vectors, such as viral vectors, encoding such interfering RNA constructs. Exemplary viral vectors described herein that encode interfering RNA constructs (e.g., siRNA, miRNA, or shRNA) for suppressing the expression of RNA transcripts containing aberrantly expanded repeat regions are adeno-associated viral (AAV) vectors, such as pseudotyped AAV2/8 and AAV2/9 vectors.

Using the compositions and methods described herein, a patient experiencing a spliceopathy and/or having a disease associated with RNA dominance, such as myotonic dystrophy, among others, can be administered a nucleic acid containing an interfering RNA construct, or a vector encoding the same, so as to reduce the expression of RNA transcripts containing expanded repeat regions and release splicing factor proteins that are sequestered by repeat-expanded RNA. For example, the compositions and methods described herein can be used to treat patients having myotonic dystrophy, as such patients may be administered an interfering RNA construct or a viral vector, such as an AAV vector, encoding such a construct, thereby reducing the expression of RNA transcripts encoding dystrophia myotonica protein kinase (DMPK). Wild-type DMPK RNA constructs typically contain from about 5 to about 37 CUG trinucleotide repeats in the 3′ untranslated region (UTR) of such transcripts. Patients having myotonic dystrophy, however, express DMPK RNA transcripts that contain 50 or more CUG repeats. The compositions and methods described herein can be used to treat patients expressing this mutant DMPK RNA, thereby releasing splicing factors to orchestrate the proper splicing of proteins associated with muscle function and treating an underlying cause of myotonic dystrophy. Similarly, the compositions and methods described herein can be used to reduce spliceopathy in, and treat one or more underlying causes of, various other disorders associated with RNA dominance and the expression of repeat-expanded RNA transcripts.

The present disclosure is based, in part, on the surprising discovery that interfering RNA constructs that anneal to repeat-expanded RNA targets at sites distal from the expanded repeat region can be used to suppress the expression of such RNA transcripts and effectively release splicing factor proteins that would otherwise be sequestered by these molecules. The compositions and methods described herein can thus attenuate the expression and nuclear retention of pathological RNA transcripts without the need to contain complementary nucleotide repeat motifs. This property provides an important clinical benefit. Nucleotide repeats are ubiquitous in mammalian genomes, such as in the genomes of human patients. The use of interfering RNA constructs that do not have nucleotide repeats, but rather anneal to other regions of a target RNA transcript, enables the selective suppression of transcripts that give rise to RNA dominance disorders without disrupting the expression of other transcripts, such as those encoding other genes that happen to contain nucleotide repeat regions but that do not aberrantly sequester splicing factor proteins. Using the compositions and methods described herein, the expression of RNA transcripts that contain pathological nucleotide repeat expansions can be diminished, while preserving the expression of important healthy RNA transcripts (for example, an RNA transcript encoding a non-target gene that happens to contain a nucleotide repeat), as well as their downstream protein products.

In a first aspect, the invention features a viral vector containing one or more transgenes encoding an interfering RNA. For example, the viral vector may contain from one to five such transgenes, from one to 10 such transgenes, from one to 15 such transgenes, from one to 20 such transgenes, from one to 50 such transgenes, from one to 100 such transgenes, from one to 1,000 such transgenes, or more (e.g., 1, 2, 3, 4, 5, 6, 7, 8, 9, 10, 20, 30, 40, 50, 100, 1,000, or more, such transgenes). The interfering RNA(s) may be at least 5, at least 10, at least 17, at least 19, or more, nucleotides in length, (e.g., at least 5, 6, 7, 8, 9, 10, 11, 12, 13, 14, 15, 16, 17, 18, 19, or more, nucleotides in length, such as from 17 to 24, 18 to 23, or 19 to 22 nucleotides in length).

The interfering RNA(s) may, e.g., each be, independently, from 10-35 nucleotides in length. In some embodiments, the interfering RNA(s) are 10 nucleotides in length. In some embodiments, the interfering RNA(s) are 11 nucleotides in length. In some embodiments, the interfering RNA(s) are 12 nucleotides in length. In some embodiments, the interfering RNA(s) are 13 nucleotides in length. In some embodiments, the interfering RNA(s) are 14 nucleotides in length. In some embodiments, the interfering RNA(s) are 15 nucleotides in length. In some embodiments, the interfering RNA(s) are 16 nucleotides in length. In some embodiments, the interfering RNA(s) are 17 nucleotides in length. In some embodiments, the interfering RNA(s) are 18 nucleotides in length. In some embodiments, the interfering RNA(s) are 19 nucleotides in length. In some embodiments, the interfering RNA(s) are 20 nucleotides in length. In some embodiments, the interfering RNA(s) are 21 nucleotides in length. In some embodiments, the interfering RNA(s) are 22 nucleotides in length. In some embodiments, the interfering RNA(s) are 23 nucleotides in length. In some embodiments, the interfering RNA(s) are 24 nucleotides in length. In some embodiments, the interfering RNA(s) are 25 nucleotides in length. In some embodiments, the interfering RNA(s) are 26 nucleotides in length. In some embodiments, the interfering RNA(s) are 27 nucleotides in length. In some embodiments, the interfering RNA(s) are 28 nucleotides in length. In some embodiments, the interfering RNA(s) are 29 nucleotides in length. In some embodiments, the interfering RNA(s) are 30 nucleotides in length. In some embodiments, the interfering RNA(s) are 31 nucleotides in length. In some embodiments, the interfering RNA(s) are 32 nucleotides in length. In some embodiments, the interfering RNA(s) are 33 nucleotides in length. In some embodiments, the interfering RNA(s) are 34 nucleotides in length. In some embodiments, the interfering RNA(s) are 35 nucleotides in length.

In some embodiments, the interfering RNA(s) contain a portion that anneals to an endogenous RNA transcript containing an expanded repeat region. The portion of each interfering RNA(s) may anneal to a segment of the endogenous RNA transcript that does not overlap with the expanded repeat region.

In some embodiments, the endogenous RNA transcript encodes human DMPK and contains an expanded repeat region. The expanded repeat region may contain, for example, 50 or more CUG trinucleotide repeats, such as from about 50 to about 4,000 CUG trinucleotide repeats (e.g., about 50 CUG trinucleotide repeats, about 60 CUG trinucleotide repeats, about 70 trinucleotide repeats, 80 trinucleotide repeats, 90 trinucleotide repeats, 100 trinucleotide repeats, 110 trinucleotide repeats, 120 trinucleotide repeats, 130 trinucleotide repeats, 140 trinucleotide repeats, 150 trinucleotide repeats, 160 trinucleotide repeats, 170 trinucleotide repeats, 180 trinucleotide repeats, 190 trinucleotide repeats, 200 trinucleotide repeats, 210 trinucleotide repeats, 220 trinucleotide repeats, 230 trinucleotide repeats, 240 trinucleotide repeats, 250 trinucleotide repeats, 260 trinucleotide repeats, 270 trinucleotide repeats, 280 trinucleotide repeats, 290 trinucleotide repeats, 300 trinucleotide repeats, 310 trinucleotide repeats, 320 trinucleotide repeats, 330 trinucleotide repeats, 340 trinucleotide repeats, 350 trinucleotide repeats, 360 trinucleotide repeats, 370 trinucleotide repeats, 380 trinucleotide repeats, 390 trinucleotide repeats, 400 trinucleotide repeats, 410 trinucleotide repeats, 420 trinucleotide repeats, 430 trinucleotide repeats, 440 trinucleotide repeats, 450 trinucleotide repeats, 460 trinucleotide repeats, 470 trinucleotide repeats, 480 trinucleotide repeats, 490 trinucleotide repeats, 500 trinucleotide repeats, 510 trinucleotide repeats, 520 trinucleotide repeats, 530 trinucleotide repeats, 540 trinucleotide repeats, 550 trinucleotide repeats, 560 trinucleotide repeats, 570 trinucleotide repeats, 580 trinucleotide repeats, 590 trinucleotide repeats, 600 trinucleotide repeats, 610 trinucleotide repeats, 620 trinucleotide repeats, 630 trinucleotide repeats, 640 trinucleotide repeats, 650 trinucleotide repeats, 660 trinucleotide repeats, 670 trinucleotide repeats, 680 trinucleotide repeats, 690 trinucleotide repeats, 700 trinucleotide repeats, 710 trinucleotide repeats, 720 trinucleotide repeats, 730 trinucleotide repeats, 740 trinucleotide repeats, 750 trinucleotide repeats, 760 trinucleotide repeats, 770 trinucleotide repeats, 780 trinucleotide repeats, 790 trinucleotide repeats, 800 trinucleotide repeats, 810 trinucleotide repeats, 820 trinucleotide repeats, 830 trinucleotide repeats, 840 trinucleotide repeats, 850 trinucleotide repeats, 860 trinucleotide repeats, 870 trinucleotide repeats, 880 trinucleotide repeats, 890 trinucleotide repeats, 900 trinucleotide repeats, 910 trinucleotide repeats, 920 trinucleotide repeats, 930 trinucleotide repeats, 940 trinucleotide repeats, 950 trinucleotide repeats, 960 trinucleotide repeats, 970 trinucleotide repeats, 980 trinucleotide repeats, 990 trinucleotide repeats, 1,000 trinucleotide repeats, 1,100 trinucleotide repeats, 1,200 trinucleotide repeats, 1,300 trinucleotide repeats, 1,400 trinucleotide repeats, 1,500 trinucleotide repeats, 1,600 trinucleotide repeats, 1,700 trinucleotide repeats, 1,800 trinucleotide repeats, 1,900 trinucleotide repeats, 2,000 trinucleotide repeats, 2,100 trinucleotide repeats, 2,200 trinucleotide repeats, 2,300 trinucleotide repeats, 2,400 trinucleotide repeats, 2,500 trinucleotide repeats, 2,600 trinucleotide repeats, 2,700 trinucleotide repeats, 2,800 trinucleotide repeats, 2,900 trinucleotide repeats, 3,000 trinucleotide repeats, 3,100 trinucleotide repeats, 3,200 trinucleotide repeats, 3,300 trinucleotide repeats, 3,400 trinucleotide repeats, 3,500 trinucleotide repeats, 3,600 trinucleotide repeats, 3,700 trinucleotide repeats, 3,800 trinucleotide repeats, 3,900 trinucleotide repeats, or 4,000 trinucleotide repeats, among others). In some embodiments, the endogenous RNA transcript contains a portion having at least 85% sequence identity (e.g., 85%, 85%, 87%, 88%, 89%, 90%, 91%, 92%, 93%, 94%, 95%, 96%, 97%, 98%, 99%, 99.9%, or 100% sequence identity) to the nucleic acid sequence of SEQ ID NO: 1 or SEQ ID NO: 2. In some embodiments, the endogenous RNA transcript contains a portion having at least 90% sequence identity (e.g., 90%, 91%, 92%, 93%, 94%, 95%, 96%, 97%, 98%, 99%, 99.9%, or 100% sequence identity) to the nucleic acid sequence of SEQ ID NO: 1 or SEQ ID NO: 2. In some embodiments, the endogenous RNA transcript contains a portion having at least 95% sequence identity (e.g., 95%, 96%, 97%, 98%, 99%, 99.9%, or 100% sequence identity) to the nucleic acid sequence of SEQ ID NO: 1 or SEQ ID NO: 2. The endogenous RNA transcript may contain, for example, a portion having the nucleic acid sequence of SEQ ID NO: 1 or SEQ ID NO: 2.

In some embodiments, the viral vector further comprises a transgene encoding human DMPK, such as a codon-optimized human DMPK that, upon transcription, does not anneal to the interfering RNA. For example, the DMPK transcript expressed by the transgene encoding human DMPK may be less than 85% complementary to the interfering RNA (e.g., less than 85%, 80%, 75%, 70%, 65%, 60%, 55%, 50%, 45%, 40%, 35%, 30%, 25%, 20%, 15%, 10%, 5%, 4%, 3%, 2%, or 1% complementary, or less). The transgene encoding human DMPK may be operably linked to the transgene(s) encoding the interfering RNA, for example, such that the interfering RNA(s) and the DMPK are expressed from the same promoter. This may be effectuated, for instance, by the placement of an internal ribosomal entry site (IRES) between the transgene(s) encoding the interfering RNA(s) and the transgene encoding human DMPK. In some embodiments, the transgene(s) encoding the interfering RNA(s) and the transgene encoding human DMPK are each operably linked to separate promoters.

In some embodiments, the portion of each interfering RNA anneals to a segment of the endogenous RNA transcript having the nucleic acid sequence of any one of SEQ ID NOs: 3-39.

In some embodiments, the portion of each interfering RNA anneals to a segment of the endogenous RNA transcript within any one of exons 1-15 of human DMPK RNA (e.g., to a segment within exon 1, exon 2, exon 3, exon 4, exon 5, exon 6, exon 7, exon 8, exon 9, exon 10, exon 11, exon 12, exon 13, exon 14, or exon 15 of human DMPK RNA). The portion of each interfering RNA may have, for example, a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment within any one of exons 1-15 of human DMPK. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment within any one of exons 1-15 of human DMPK. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment within any one of exons 1-15 of human DMPK. In some embodiments, the portion of each interfering RNA has a nucleic acid sequence that is completely complementary to the nucleic acid sequence of a segment within any one of exons 1-15 of human DMPK.

In some embodiments, the portion of each interfering RNA anneals to a segment of the endogenous RNA transcript within any one of introns 1-14 of human DMPK RNA (e.g., to a segment within intron 1, intron 2, intron 3, intron 4, intron 5, intron 6, intron 7, intron 8, intron 9, intron 10, intron 11, intron 12, intron 13, or intron 14 of human DMPK RNA). The portion of each interfering RNA may have, for example, a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment within any one of introns 1-14 human DMPK. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment within any one of introns 1-14 of human DMPK. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment within any one of introns 1-14 of human DMPK. In some embodiments, the portion of each interfering RNA has a nucleic acid sequence that is completely complementary to the nucleic acid sequence of a segment within any one of introns 1-14 of human DMPK.

In some embodiments, the portion of each interfering RNA anneals to a segment of the endogenous RNA transcript containing an exon-intron boundary within human DMPK (e.g., to a segment containing the boundary between exon 1 and intron 1, between intron 1 and exon 2, between exon 2 and intron 2, between intron 2 and exon 3, between exon 3 and intron 3, between intron 3 and exon 4, between exon 4 and intron 4, between intron 4 and exon 5, between exon 5 and intron 5, between intron 5 and exon 6, between exon 6 and intron 6, between intron 6 and exon 7, between exon 7 and intron 7, between intron 7 and exon 8, between exon 8 and intron 8, between intron 8 and exon 9, between exon 9 and intron 9, between intron 9 and exon 10, between exon 10 and intron 10, between intron 10 and exon 11, between exon 11 and intron 11, between intron 11 and exon 12, between exon 12 and intron 12, between intron 12 and exon 13, between exon 13 and intron 13, between intron 13 and exon 14, between exon 14 and intron 14, or between intron 14 and exon 15). The portion of each interfering RNA may have, for example, a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment containing an exon-intron boundary within human DMPK. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment containing an exon-intron boundary within human DMPK. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment containing an exon-intron boundary within human DMPK. In some embodiments, the portion of each interfering RNA has a nucleic acid sequence that is completely complementary to the nucleic acid sequence of a segment containing an exon-intron boundary within human DMPK.

In some embodiments, the portion of each interfering RNA anneals to a segment of the endogenous RNA transcript within the 5′ UTR or 3′ UTR of human DMPK. The portion of each interfering RNA may have, for example, a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment within the 5′ UTR or 3′ UTR of human DMPK. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment within the 5′ UTR or 3′ UTR of human DMPK. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment within the 5′ UTR or 3′ UTR of human DMPK. In some embodiments, the portion of each interfering RNA has a nucleic acid sequence that is completely complementary to the nucleic acid sequence of a segment within the 5′ UTR or 3′ UTR of human DMPK.

In some embodiments, the segment within human DMPK is from about 10 to about 80 nucleotides in length. For example, the segment may be 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, or 80 nucleotides in length. In some embodiments, the segment within human DMPK is from about 15 to about 50 nucleotides in length, such as a segment of 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides in length. In some embodiments, the segment within human DMPK is from about 17 to about 23 nucleotides in length, such as 17 nucleotides, 18 nucleotides, 19 nucleotides, 20 nucleotides, 21 nucleotides, 22 nucleotides, or 23 nucleotides in length. In some embodiments, the segment is 18 nucleotides in length. In some embodiments, the segment is 19 nucleotides in length. In some embodiments, the segment is 20 nucleotides in length. In some embodiments, the segment is 21 nucleotides in length.

In some embodiments, the interfering RNA anneals to the endogenous RNA transcript encoding human DMPK with from one to eight nucleotide mismatches (e.g., with one nucleotide mismatch, two nucleotide mismatches, three nucleotide mismatches, four nucleotide mismatches, five nucleotide mismatches, six nucleotide mismatches, seven nucleotide mismatches, or eight nucleotide mismatches). In some embodiments, the interfering RNA anneals to the endogenous RNA transcript encoding human DMPK with from one to five nucleotide mismatches, such as with one nucleotide mismatch, two nucleotide mismatches, three nucleotide mismatches, four nucleotide mismatches, or five nucleotide mismatches. In some embodiments, the interfering RNA anneals to the endogenous RNA transcript encoding human DMPK with from one to three nucleotide mismatches, such as with one nucleotide mismatch, two nucleotide mismatches, or three nucleotide mismatches. In some embodiments, the interfering RNA anneals to the endogenous RNA transcript encoding human DMPK with no more than two nucleotide mismatches. For example, interfering RNA may anneal to the endogenous RNA transcript encoding human DMKPK with no nucleotide mismatches, one nucleotide mismatch, or two nucleotide mismatches.

In some embodiments, the interfering RNA contains a portion having at least 85% sequence identity (e.g., 85%, 85%, 87%, 88%, 89%, 90%, 91%, 92%, 93%, 94%, 95%, 96%, 97%, 98%, 99%, 99.9%, or 100% sequence identity) to the nucleic acid sequence of any one of SEQ ID NOs: 3-161. The interfering RNA may contain, for example, a portion having at least 90% sequence identity (e.g., 90%, 91%, 92%, 93%, 94%, 95%, 96%, 97%, 98%, 99%, 99.9%, or 100% sequence identity) to the nucleic acid sequence of ay one of SEQ ID NOs: 3-161. In some embodiments, the interfering RNA contains a portion having at least 95% sequence identity (e.g., 95%, 96%, 97%, 98%, 99%, 99.9%, or 100% sequence identity) to the nucleic acid sequence of ay one of SEQ ID NOs: 3-161. In some embodiments, the interfering RNA contains a portion having the nucleic acid sequence of any one of SEQ ID NOs: 3-161. In some embodiments, the interfering RNA is a miRNA having a combination of passenger and guide strands shown in Table 5, herein.

In some embodiments, the endogenous RNA transcript contains human chromosome 9 open reading frame 72 (C9ORF72) and an expanded repeat region. The expanded repeat region may contain, for example, greater than 25 GGGGCC (SEQ ID NO: 162) hexanucleotide repeats, such as from about 700 to about 1,600 GGGGCC (SEQ ID NO: 162) hexanucleotide repeats, For example, the expanded repeat region may contain 700 hexanucleotide repeats, 710 hexanucleotide repeats, 720 hexanucleotide repeats, 730 hexanucleotide repeats, 740 hexanucleotide repeats, 750 hexanucleotide repeats, 760 hexanucleotide repeats, 770 hexanucleotide repeats, 780 hexanucleotide repeats, 790 hexanucleotide repeats, 800 hexanucleotide repeats, 810 hexanucleotide repeats, 820 hexanucleotide repeats, 830 hexanucleotide repeats, 840 hexanucleotide repeats, 850 hexanucleotide repeats, 860 hexanucleotide repeats, 870 hexanucleotide repeats, 880 hexanucleotide repeats, 890 hexanucleotide repeats, 900 hexanucleotide repeats, 910 hexanucleotide repeats, 920 hexanucleotide repeats, 930 hexanucleotide repeats, 940 hexanucleotide repeats, 950 hexanucleotide repeats, 960 hexanucleotide repeats, 970 hexanucleotide repeats, 980 hexanucleotide repeats, 990 hexanucleotide repeats, or 1,000 hexanucleotide repeats, among others. The expanded repeat region may contain greater than 30 hexanucleotide repeats, such as 30 hexanucleotide repeats, 40 hexanucleotide repeats, 50 hexanucleotide repeats, 60 CUG hexanucleotide repeats, 70 hexanucleotide repeats, 80 hexanucleotide repeats, 90 hexanucleotide repeats, 100 hexanucleotide repeats, 110 hexanucleotide repeats, 120 hexanucleotide repeats, 130 hexanucleotide repeats, 140 hexanucleotide repeats, 150 hexanucleotide repeats, 160 hexanucleotide repeats, 170 hexanucleotide repeats, 180 hexanucleotide repeats, 190 hexanucleotide repeats, 200 hexanucleotide repeats, 210 hexanucleotide repeats, 220 hexanucleotide repeats, 230 hexanucleotide repeats, 240 hexanucleotide repeats, 250 hexanucleotide repeats, 260 hexanucleotide repeats, 270 hexanucleotide repeats, 280 hexanucleotide repeats, 290 hexanucleotide repeats, 300 hexanucleotide repeats, 310 hexanucleotide repeats, 320 hexanucleotide repeats, 330 hexanucleotide repeats, 340 hexanucleotide repeats, 350 hexanucleotide repeats, 360 hexanucleotide repeats, 370 hexanucleotide repeats, 380 hexanucleotide repeats, 390 hexanucleotide repeats, 400 hexanucleotide repeats, 410 hexanucleotide repeats, 420 hexanucleotide repeats, 430 hexanucleotide repeats, 440 hexanucleotide repeats, 450 hexanucleotide repeats, 460 hexanucleotide repeats, 470 hexanucleotide repeats, 480 hexanucleotide repeats, 490 hexanucleotide repeats, 500 hexanucleotide repeats, 510 hexanucleotide repeats, 520 hexanucleotide repeats, 530 hexanucleotide repeats, 540 hexanucleotide repeats, 550 hexanucleotide repeats, 560 hexanucleotide repeats, 570 hexanucleotide repeats, 580 hexanucleotide repeats, 590 hexanucleotide repeats, 600 hexanucleotide repeats, 610 hexanucleotide repeats, 620 hexanucleotide repeats, 630 hexanucleotide repeats, 640 hexanucleotide repeats, 650 hexanucleotide repeats, 660 hexanucleotide repeats, 670 hexanucleotide repeats, 680 hexanucleotide repeats, 690 hexanucleotide repeats, 700 hexanucleotide repeats, 710 hexanucleotide repeats, 720 hexanucleotide repeats, 730 hexanucleotide repeats, 740 hexanucleotide repeats, 750 hexanucleotide repeats, 760 hexanucleotide repeats, 770 hexanucleotide repeats, 780 hexanucleotide repeats, 790 hexanucleotide repeats, 800 hexanucleotide repeats, 810 hexanucleotide repeats, 820 hexanucleotide repeats, 830 hexanucleotide repeats, 840 hexanucleotide repeats, 850 hexanucleotide repeats, 860 hexanucleotide repeats, 870 hexanucleotide repeats, 880 hexanucleotide repeats, 890 hexanucleotide repeats, 900 hexanucleotide repeats, 910 hexanucleotide repeats, 920 hexanucleotide repeats, 930 hexanucleotide repeats, 940 hexanucleotide repeats, 950 hexanucleotide repeats, 960 hexanucleotide repeats, 970 hexanucleotide repeats, 980 hexanucleotide repeats, 990 hexanucleotide repeats, 1,000 hexanucleotide repeats, 1,100 hexanucleotide repeats, 1,200 hexanucleotide repeats, 1,300 hexanucleotide repeats, 1,400 hexanucleotide repeats, 1,500 hexanucleotide repeats, 1,600 hexanucleotide repeats, or more.

In some embodiments, the endogenous RNA transcript contains a portion having at least 85% sequence identity (e.g., 85%, 85%, 87%, 88%, 89%, 90%, 91%, 92%, 93%, 94%, 95%, 96%, 97%, 98%, 99%, 99.9%, or 100% sequence identity) to the nucleic acid sequence of SEQ ID NO: 163. The endogenous RNA transcript may contain, for example, a portion having at least 90% sequence identity (e.g., 90%, 91%, 92%, 93%, 94%, 95%, 96%, 97%, 98%, 99%, 99.9%, or 100% sequence identity) to the nucleic acid sequence of SEQ ID NO: 163. In some embodiments, the endogenous RNA transcript contains a portion having at least 95% sequence identity (e.g., 95%, 96%, 97%, 98%, 99%, 99.9%, or 100% sequence identity) to the nucleic acid sequence of SEQ ID NO: 163. In some embodiments, the RNA transcript contains a portion having the nucleic acid sequence of SEQ ID NO: 163.

In some embodiments, the endogenous RNA transcript contains a portion having at least 85% sequence identity (e.g., 85%, 85%, 87%, 88%, 89%, 90%, 91%, 92%, 93%, 94%, 95%, 96%, 97%, 98%, 99%, 99.9%, or 100% sequence identity) to the nucleic acid sequence of SEQ ID NO: 165. The endogenous RNA transcript may contain, for example, a portion having at least 90% sequence identity (e.g., 90%, 91%, 92%, 93%, 94%, 95%, 96%, 97%, 98%, 99%, 99.9%, or 100% sequence identity) to the nucleic acid sequence of SEQ ID NO: 165. In some embodiments, the endogenous RNA transcript contains a portion having at least 95% sequence identity (e.g., 95%, 96%, 97%, 98%, 99%, 99.9%, or 100% sequence identity) to the nucleic acid sequence of SEQ ID NO: 165. In some embodiments, the RNA transcript contains a portion having the nucleic acid sequence of SEQ ID NO: 165.

In some embodiments, the endogenous RNA transcript contains a portion having at least 85% sequence identity (e.g., 85%, 85%, 87%, 88%, 89%, 90%, 91%, 92%, 93%, 94%, 95%, 96%, 97%, 98%, 99%, 99.9%, or 100% sequence identity) to the nucleic acid sequence of SEQ ID NO: 166. The endogenous RNA transcript may contain, for example, a portion having at least 90% sequence identity (e.g., 90%, 91%, 92%, 93%, 94%, 95%, 96%, 97%, 98%, 99%, 99.9%, or 100% sequence identity) to the nucleic acid sequence of SEQ ID NO: 166. In some embodiments, the endogenous RNA transcript contains a portion having at least 95% sequence identity (e.g., 95%, 96%, 97%, 98%, 99%, 99.9%, or 100% sequence identity) to the nucleic acid sequence of SEQ ID NO: 166. In some embodiments, the RNA transcript contains a portion having the nucleic acid sequence of SEQ ID NO: 166.

In some embodiments, the portion of each interfering RNA has a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment within human C9ORF72, such as to the nucleic acid sequence of a segment within SEQ ID NO: 163, 165, or 166. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment within human C9ORF72, such as to the nucleic acid sequence of a segment within SEQ ID NO: 163, 165, or 166. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment within human C9ORF72, such as to the nucleic acid sequence of a segment within SEQ ID NO: 163, 165, or 166. In some embodiments, the portion of each interfering RNA has a nucleic acid sequence that is completely complementary to the nucleic acid sequence of a segment within human C9ORF72, such as to the nucleic acid sequence of a segment within SEQ ID NO: 163, 165, or 166.

In some embodiments, the segment within human C9ORF72 is from about 10 to about 80 nucleotides in length. For example, the segment may be 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, or 80 nucleotides in length. In some embodiments, the segment within human C9ORF72 is from about 15 to about 50 nucleotides in length, such as a segment of 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides in length. In some embodiments, the segment within human C9ORF72 is from about 17 to about 23 nucleotides in length, such as 17 nucleotides, 18 nucleotides, 19 nucleotides, 20 nucleotides, 21 nucleotides, 22 nucleotides, or 23 nucleotides in length. In some embodiments, the segment is 18 nucleotides in length. In some embodiments, the segment is 19 nucleotides in length. In some embodiments, the segment is 20 nucleotides in length. In some embodiments, the segment is 21 nucleotides in length.

In some embodiments, the interfering RNA anneals to the endogenous RNA transcript encoding human C9ORF72 with from one to eight nucleotide mismatches (e.g., with one nucleotide mismatch, two nucleotide mismatches, three nucleotide mismatches, four nucleotide mismatches, five nucleotide mismatches, six nucleotide mismatches, seven nucleotide mismatches, or eight nucleotide mismatches). In some embodiments, the interfering RNA anneals to the endogenous RNA transcript encoding human C9ORF72 with from one to five nucleotide mismatches, such as with one nucleotide mismatch, two nucleotide mismatches, three nucleotide mismatches, four nucleotide mismatches, or five nucleotide mismatches. In some embodiments, the interfering RNA anneals to the endogenous RNA transcript encoding human C9ORF72 with from one to three nucleotide mismatches, such as with one nucleotide mismatch, two nucleotide mismatches, or three nucleotide mismatches. In some embodiments, the interfering RNA anneals to the endogenous RNA transcript encoding human C9ORF72 with no more than two nucleotide mismatches. For example, interfering RNA may anneal to the endogenous RNA transcript encoding human C9ORF72 with no nucleotide mismatches, one nucleotide mismatch, or two nucleotide mismatches.

In some embodiments, the interfering RNA is a short interfering RNA (siRNA), a short hairpin RNA (shRNA), or a micro RNA (miRNA), such as a U6 miRNA. The miRNA may be based, for example, on the endogenous human miR30a nucleic acid sequence, having one or more nucleic acid substitutions as needed for complementarity to a target mRNA (e.g., a target mRNA described herein). In the case of a miRNA, the viral vector may contain, for example, a primary miRNA (pri-miRNA) transcript encoding a mature miRNA. In some embodiments, the viral vector contains a pre-miRNA transcript encoding a mature miRNA.

In some embodiments, the interfering RNA is operably linked to a promoter that induces expression of the interfering RNA in a muscle cell or neuron. The promoter may be, for example, a desmin promoter, a phosphoglycerate kinase (PGK) promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3 (PITX3).

In some embodiments, the viral vector is an AAV, adenovirus, lentivirus, retrovirus, poxvirus, baculovirus, herpes simplex virus, vaccinia virus, or a synthetic virus. The viral vector may be, for example, an AAV1, AAV2, AAV3, AAV4, AAV5, AAV6, AAV7, AAV8, AAV9, AAVrh10, or AAVrh74 serotype. In some embodiments, the viral vector is a pseudotyped AAV, such as an AAV2/8 or AAV/29 vector. The viral vector may contain a recombinant capsid protein. In some embodiments, the viral vector is a synthetic virus, such as a chimeric virus, mosaic virus, or pseudotyped virus, and/or a synthetic virus that contains a foreign protein, synthetic polymer, nanoparticle, or small molecule.

In another aspect, the invention features a nucleic acid encoding or containing an interfering RNA that contains a portion having at least 85% sequence identity (e.g., 85%, 85%, 87%, 88%, 89%, 90%, 91%, 92%, 93%, 94%, 95%, 96%, 97%, 98%, 99%, 99.9%, or 100% sequence identity) to the nucleic acid sequence of any one of SEQ ID NOs: 3-161. In some embodiments, the interfering RNA contains a portion having at least 90% sequence identity (e.g., 90%, 91%, 92%, 93%, 94%, 95%, 96%, 97%, 98%, 99%, 99.9%, or 100% sequence identity) to the nucleic acid sequence of any one of SEQ ID NOs: 3-161. The interfering RNA may contain, for example, a portion having at least 95% sequence identity (e.g., 95%, 96%, 97%, 98%, 99%, 99.9%, or 100% sequence identity) to the nucleic acid sequence of any one of SEQ ID NOs: 3-161. In some embodiments, the interfering RNA contains a portion having the nucleic acid sequence of any one of SEQ ID NOs: 3-161. In some embodiments, the interfering RNA is a miRNA having a combination of passenger and guide strands shown in Table 5, herein.

In some embodiments, the portion of each interfering RNA anneals to a segment of an endogenous RNA transcript encoding human DMPK within any one of exons 1-15 of human DMPK RNA (e.g., to a segment within exon 1, exon 2, exon 3, exon 4, exon 5, exon 6, exon 7, exon 8, exon 9, exon 10, exon 11, exon 12, exon 13, exon 14, or exon 15 of human DMPK RNA). The portion of each interfering RNA may have, for example, a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment within any one of exons 1-15 of human DMPK. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment within any one of exons 1-15 of human DMPK. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment within any one of exons 1-15 of human DMPK. In some embodiments, the portion of each interfering RNA has a nucleic acid sequence that is completely complementary to the nucleic acid sequence of a segment within any one of exons 1-15 of human DMPK.

In some embodiments, the portion of each interfering RNA anneals to a segment of an endogenous RNA transcript encoding human DMPK within any one of introns 1-14 of human DMPK RNA (e.g., to a segment within intron 1, intron 2, intron 3, intron 4, intron 5, intron 6, intron 7, intron 8, intron 9, intron 10, intron 11, intron 12, intron 13, or intron 14 of human DMPK RNA). The portion of each interfering RNA may have, for example, a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment within any one of introns 1-14 human DMPK. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment within any one of introns 1-14 of human DMPK. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment within any one of introns 1-14 of human DMPK. In some embodiments, the portion of each interfering RNA has a nucleic acid sequence that is completely complementary to the nucleic acid sequence of a segment within any one of introns 1-14 of human DMPK.

In some embodiments, the portion of each interfering RNA anneals to a segment of an endogenous RNA transcript encoding human DMPK containing an exon-intron boundary within human DMPK (e.g., to a segment containing the boundary between exon 1 and intron 1, between intron 1 and exon 2, between exon 2 and intron 2, between intron 2 and exon 3, between exon 3 and intron 3, between intron 3 and exon 4, between exon 4 and intron 4, between intron 4 and exon 5, between exon 5 and intron 5, between intron 5 and exon 6, between exon 6 and intron 6, between intron 6 and exon 7, between exon 7 and intron 7, between intron 7 and exon 8, between exon 8 and intron 8, between intron 8 and exon 9, between exon 9 and intron 9, between intron 9 and exon 10, between exon 10 and intron 10, between intron 10 and exon 11, between exon 11 and intron 11, between intron 11 and exon 12, between exon 12 and intron 12, between intron 12 and exon 13, between exon 13 and intron 13, between intron 13 and exon 14, between exon 14 and intron 14, or between intron 14 and exon 15). The portion of each interfering RNA may have, for example, a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment containing an exon-intron boundary within human DMPK. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment containing an exon-intron boundary within human DMPK. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment containing an exon-intron boundary within human DMPK. In some embodiments, the portion of each interfering RNA has a nucleic acid sequence that is completely complementary to the nucleic acid sequence of a segment containing an exon-intron boundary within human DMPK.

In some embodiments, the portion of each interfering RNA anneals to a segment of an endogenous RNA transcript encoding human DMPK within the 5′ UTR or 3′ UTR of human DMPK. The portion of each interfering RNA may have, for example, a nucleic acid sequence that is least 85% complementary (e.g., 85%, 85%, 87%, 88%, 89%, 90%, 91%, 92%, 93%, 94%, 95%, 96%, 97%, 98%, 99%, 99.9%, or 100% complementary) to the nucleic acid sequence of a segment within the 5′ UTR or 3′ UTR of human DMPK. In some embodiments, the portion of each interfering RNA has a nucleic acid sequence that is at least 90% complementary (e.g., 90%, 91%, 92%, 93%, 94%, 95%, 96%, 97%, 98%, 99%, 99.9%, or 100% complementary) to the nucleic acid sequence of a segment within the 5′ UTR or 3′ UTR of human DMPK. For example, the portion of each interfering RNA may have a nucleic acid sequence that is at least 95% complementary (e.g., 95%, 96%, 97%, 98%, 99%, 99.9%, or 100% complementary) to the nucleic acid sequence of a segment within the 5′ UTR or 3′ UTR of human DMPK. In some embodiments, the portion of each interfering RNA has a nucleic acid sequence that is completely complementary to the nucleic acid sequence of a segment within the 5′ UTR or 3′ UTR of human DMPK.

In some embodiments, the segment within human DMPK is from about 10 to about 80 nucleotides in length. For example, the segment may be 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, 50 nucleotides, 51 nucleotides, 52 nucleotides, 53 nucleotides, 54 nucleotides, 55 nucleotides, 56 nucleotides, 57 nucleotides, 58 nucleotides, 59 nucleotides, 60 nucleotides, 61 nucleotides, 62 nucleotides, 63 nucleotides, 64 nucleotides, 65 nucleotides, 66 nucleotides, 67 nucleotides, 68 nucleotides, 69 nucleotides, 70 nucleotides, 71 nucleotides, 72 nucleotides, 73 nucleotides, 74 nucleotides, 75 nucleotides, 76 nucleotides, 77 nucleotides, 78 nucleotides, 79 nucleotides, or 80 nucleotides in length. In some embodiments, the segment within human DMPK is from about 15 to about 50 nucleotides in length, such as a segment of 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides, or 50 nucleotides in length. In some embodiments, the segment within human DMPK is from about 17 to about 23 nucleotides in length, such as 17 nucleotides, 18 nucleotides, 19 nucleotides, 20 nucleotides, 21 nucleotides, 22 nucleotides, or 23 nucleotides in length. In some embodiments, the segment is 18 nucleotides in length. In some embodiments, the segment is 19 nucleotides in length. In some embodiments, the segment is 20 nucleotides in length. In some embodiments, the segment is 21 nucleotides in length.

In some embodiments, the interfering RNA anneals to an endogenous RNA transcript encoding human DMPK with from one to eight nucleotide mismatches (e.g., with one nucleotide mismatch, two nucleotide mismatches, three nucleotide mismatches, four nucleotide mismatches, five nucleotide mismatches, six nucleotide mismatches, seven nucleotide mismatches, or eight nucleotide mismatches). In some embodiments, the interfering RNA anneals to an endogenous RNA transcript encoding human DMPK with from one to five nucleotide mismatches, such as with one nucleotide mismatch, two nucleotide mismatches, three nucleotide mismatches, four nucleotide mismatches, or five nucleotide mismatches. In some embodiments, the interfering RNA anneals to an endogenous RNA transcript encoding human DMPK with from one to three nucleotide mismatches, such as with one nucleotide mismatch, two nucleotide mismatches, or three nucleotide mismatches. In some embodiments, the interfering RNA anneals to an endogenous RNA transcript encoding human DMPK with no more than two nucleotide mismatches. For example, interfering RNA may anneal to the endogenous RNA transcript encoding human DMKPK with no nucleotide mismatches, one nucleotide mismatch, or two nucleotide mismatches.

In some embodiments, the interfering RNA is a siRNA, a shRNA, or a miRNA, such as a U6 miRNA. The miRNA may be based, for example, on the endogenous human miR30a nucleic acid sequence, having one or more nucleic acid substitutions as needed for complementarity to a target mRNA (e.g., a target mRNA described herein). In the case of a miRNA, the nucleic acid may contain, for example, a pri-miRNA transcript encoding a mature miRNA. In some embodiments, the viral vector contains a pre-miRNA transcript encoding a mature miRNA.

In some embodiments, the interfering RNA is operably linked to a promoter that induces expression of the interfering RNA in a muscle cell or neuron. The promoter may be, for example, a desmin promoter, a PGK promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular PITX3.

In an additional aspect, the invention features a vector containing the nucleic acid of any of the above aspects or embodiments. The vector may be, for example, an AAV (e.g., an AAV1, AAV2, AAV3, AAV4, AAV5, AAV6, AAV7, AAV8, AAV9, AAVrh10, or AAVrh74 serotype, or a pseudotyped AAV, such as an AAV2/8 or AAV/29 vector), adenovirus, lentivirus, retrovirus, poxvirus, baculovirus, herpes simplex virus, vaccinia virus, or a synthetic virus (e.g., a chimeric virus, mosaic virus, or pseudotyped virus, and/or a synthetic virus that contains a foreign protein, synthetic polymer, nanoparticle, or small molecule), and may contain one or more recombinant capsid proteins.

In yet another aspect, the invention features a composition containing the nucleic acid of any of the above aspects or embodiments. The composition may be, for example, a liposome, vesicle, synthetic vesicle, exosome, synthetic exosome, dendrimer, or nanoparticle.

In another aspect, the invention features a pharmaceutical composition containing the nucleic acid of any of the above aspects or embodiments. The pharmaceutical composition may further contain a pharmaceutically acceptable carrier, diluent, or excipient.

In an additional aspect, the invention features a method of reducing the occurrence of spliceopathy (e.g., of an mRNA transcript for which splicing is regulated, in part, by the activity of muscleblind-like protein) in a patient, such as a human patient, in need thereof. The method may include administering to the patient a therapeutically effective amount of the vector or composition of any of the above aspects or embodiments. In some embodiments, the patient has myotonic dystrophy. Upon administration of the vector or composition to the patient, the patient may exhibit an increase in corrective splicing of one or more RNA transcript substrates of muscleblind-like protein.

In another aspect, the invention features a method of treating a disorder characterized by nuclear retention of RNA containing an expanded repeat region in a patient, such as a human patient, in need thereof by administering to the patient a therapeutically effective amount of the vector or composition of any of the above aspects or embodiments. The disorder may be, for example, myotonic dystrophy, and the nuclear-retained RNA may be DMPK RNA. In some embodiments, the disorder is amyotrophic lateral sclerosis and the nuclear-retrained RNA is C9ORF72 RNA.

Upon administration of the vector or composition to the patient, the patient may exhibit an increase in corrective splicing of one or more RNA transcript substrates of muscleblind-like protein. For example, upon administration of the vector or composition to the patient, the patient may exhibit an increase in expression of sarcoplasmic/endoplasmic reticulum calcium ATPase 1 (SERCA1) mRNA containing exon 22, such as an increase of about 1.1-fold to about 10-fold, or more (e.g., an increase in expression of SERCA1 mRNA containing exon 22 by about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, or more), as assessed, for example, using an RNA or protein detection assay described herein.

In some embodiments, upon administration of the vector or composition to the patient, the patient may exhibit a decrease in expression of chloride voltage-gated channel 1 (CLCN1) mRNA containing exon 7a, such as a decrease of about 1% to about 100% (e.g., a decrease in expression of CLCN1 mRNA containing exon 7a by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%), as assessed, for example, using an RNA or protein detection assay described herein.

For example, upon administration of the vector or composition to the patient, the patient may exhibit a decrease in expression of ZO-2 associated speckle protein (ZASP) containing exon 11, such as a decrease of about 1% to about 100% (e.g., a decrease in expression of ZASP mRNA containing exon 11 by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%), as assessed, for example, using an RNA or protein detection assay described herein.

In some embodiments, upon administration of the vector or composition to the patient, the patient exhibits an increase in corrective splicing of RNA transcripts encoding insulin receptor, ryanodine receptor 1 (RYR1), cardiac muscle troponin, and/or skeletal muscle troponin, such as an increase of about 1.1-fold to about 10-fold, or more (e.g., an increase in expression of correctly spliced RNA transcripts encoding insulin receptor, RYR1, cardiac muscle troponin, and/or skeletal muscle troponin by about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, or more), as assessed, for example, using an RNA or protein detection assay described herein.

In some embodiments of either of the preceding two preceding aspects, the vector or composition is administered to the patient by way of intravenous, intrathecal, intracerebroventricular, intraparenchymal, intracisternal, intradermal, transdermal, parenteral, intramuscular, intranasal, subcutaneous, percutaneous, intratracheal, intraperitoneal, intraarterial, intravascular, inhalation, perfusion, lavage, or oral administration.

In another aspect, the invention features a kit containing the vector or composition of any of the above aspects or embodiments. The kit may further contain a package insert instructing a user of the kit to administer the vector or composition to the patient to reduce the occurrence of a spliceopathy in the patient, such as spliceopathy of an mRNA transcript for which splicing is regulated, in part, by the activity of muscleblind-like protein.

In an additional aspect, the invention features a kit containing the vector or composition of any of the above aspects or embodiments and a package insert that instructs a user of the kit to administer the vector or composition to the patient to reduce the occurrence of a spliceopathy in the patient to treat a disorder characterized by nuclear retention of RNA containing an expanded repeat region. The disorder may be, for example, myotonic dystrophy, and the nuclear-retained RNA may be DMPK RNA. In some embodiments, the disorder is amyotrophic lateral sclerosis and the nuclear-retrained RNA is C9ORF72 RNA.

### Definitions

As used herein, the term “about” refers to a value that is within 10% above or below the value being described. For example, the phrase “about 100 nucleic acid residues” refers to a value of from 90 to 110 nucleic acid residues.

As used herein, the term “anneal” refers to the formation of a stable duplex of nucleic acids by way of hybridization mediated by inter-strand hydrogen bonding, for example, according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another. The “stable duplex” formed upon the annealing of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties
of naturally-occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include, e.g., (i) G, A, V, L, I, P, and M; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the terms “dystrophia myotonica protein kinase” and its abbreviation, “DMPK,” refer to the serine/threonine kinase protein involved in the regulation of skeletal muscle structure and function, for example, in human subjects. The terms “dystrophia myotonica protein kinase” and “DMPK” are used interchangeably herein and refer not only to wild-type forms of the DMPK gene, but also to variants of wild-type DMPK proteins and nucleic acids encoding the same. The nucleic acid sequences of two isoforms of human DMPK mRNA are provided herein as SEQ ID NOs: 1 and 2, which correspond to GenBank Accession Nos. BC026328.1 and BC062553.1, respectively (3′ UTRs not included). These nucleic acid sequences are provided in Table 2, below.

TABLE 2
Nucleic acid sequences of exemplary human DMPK isoforms

| SEQ ID NO. | Nucleic acid sequence |
|---|---|
| 1 | GGCUGGACCAAGGGGUGGGGAGAAGGGGAGGAGGCCUCGGCCGGCCGC |
| | AGAGAGAAGUGGCCAGAGAGGCCCAGGGGACAGCCAGGGACAGGCAGAC |
| | AUGCAGCCAGGGCUCCAGGGCCUGGACAGGGGCUGCCAGGCCCUGUGA |
| | CAGGAGGACCCCGAGCCCCCGGCCCGGGGAGGGGCCAUGGUGCUGCCU |
| | GUCCAACAUGUCAGCCGAGGUGCGGCUGAGGCGGCUCCAGCAGCUGGU |
| | GUUGGACCCGGGCUUCCUGGGGCUGGAGCCCCUGCUCGACCUUCUCCU |
| | GGGCGUCCACCAGGAGCUGGGCGCCUCCGAACUGGCCCAGGACAAGUAC |
| | GUGGCCGACUUCUUGCAGUGGGCCCCAAAUCCAGGGUUUUCCAAAGUGU |
| | GGUUCAAGAACCACCUGCAUCUGAAUCUAGAGCGGAGCCCAUCGUGGUG |
| | AGGCUUAAGGAGGUCCGACUGCAGAGGGACGACUUCGAGAUUCUGAAGG |
| | UGAUCGGACGCGGGGCGUUCAGCGAGGUAGCGGUAGUGAAGAUGAAGC |
| | AGACGGGCCAGGUGUAUGCCAUGAAGAUCAUGAACAAGUGGGACAUGCU |
| | GAAGAGGGGCGAGGUGUCGUGCUUCCGUGAGGAGAGGGACGUGUUGGU |
| | GAAUGGGGACCAGCGGUGGAUCACGCAGCUGCACUUCGCCUUCCAGGAU |
| | GAGAACUACCUGUACCUGGUCAUGGAGUAUUACGUGGGCGGGGACCUGC |
| | UGACACUGCUGAGCAAGUUUGGGGAGCGGAUUCCGGCCGAGAUGGCGC |
| | GCUUCUACCUGGCGGAGAUUGUCAUGGCCAUAGACUCGGUGCACCGGCU |
| | UGGCUACGUGCACAGGGACAUCAAACCCGACAACAUCCUGCUGGACCGC |
| | UGUGGCCACAUCCGCCUGGCCGACUUCGGCUCUUGCCUCAAGCUGCGG |
| | GCAGAUGGAACGGUGCGGUCGCUGGUGGCUGUGGGCACCCCAGACUAC |
| | CUGUCCCCCGAGAUCCUGCAGGCUGUGGGCGGUGGGCCUGGGACAGGC |
| | AGCUACGGGCCCGAGUGUGACUGGUGGGCGCUGGGUGUAUUCGCCUAU |
| | GAAAUGUUCUAUGGGCAGACGCCCUUCUACGCGGAUUCCACGGCGGAGA |
| | CCUAUGGCAAGAUCGUCCACUACAAGGAGCACCUCUCUCUGCCGCUGGU |
| | GGACGAAGGGGUCCCUGAGGAGGCUCGAGACUUCAUUCAGCGGUUGCU |
| | GUGUCCCCCGGAGACACGGCUGGGCCGGGGUGGAGCAGGCGACUUCCG |
| | GACACAUCCCUUCUUCUUUGGCCUCGACUGGGAUGGUCUCCGGGACAGC |
| | GUGCCCCCCUUUACACCGGAUUUCGAAGGUGCCACCGACACAUGCAACU |
| | UCGACUUGGUGGAGGACGGGCUCACUGCCAUGGUGAGCGGGGGCGGGG |
| | AGACACUGUCGGACAUUCGGGAAGGUGCGCCGCUAGGGGUCCACCUGC |
| | CUUUUGUGGGCUACUCCUACUCCUGCAUGGCCCUCAGGGACAGUGAGGU |
| | CCCAGGCCCCACACCCAUGGAACUGGAGGCCGAGCAGCUGCUUGAGCCA |
| | CACGUGCAAGCGCCCAGCCUGGAGCCCUCGGUGUCCCCACAGGAUGAAA |
| | CAGCUGAAGUGGCAGUUCCAGCGGCUGUCCCUGCGGCAGAGGCUGAGG |
| | CCGAGGUGACGCUGCGGGAGCUCCAGGAAGCCCUGGAGGAGGAGGUGC |
| | UCACCCGGCAGAGCCUGAGCCGGGAGAUGGAGGCCAUCCGCACGGACAA |
| | CCAGAACUUCGCCAGUCAACUACGCGAGGCAGAGGCUCGGAACCGGGAC |
| | CUAGAGGCACACGUCCGGCAGUUGCAGGAGCGGAUGGAGUUGCUGCAG |
| | GCAGAGGGAGCCACAGCUGUCACGGGGGUCCCCAGUCCCCGGGCCAC |
| | GGAUCCACCUUCCCAUCUAGAUGGCCCCACGGCCGUGGCUGUGGGCCA |
| | GUGCCCGCUGGUGGGGCCAGGCCCCAUGCACCGCCGCCACCUGCUGCU |
| | CCCUGCCAGGGUCCCUAGGCCUGGCCUAUCGGAGGCGCUUUCCCUGCU |
| | CCUGUUCGCCGUUGUUCUGUCUCGUGCCGCCGCCCUGGGCUGCAUUGG |
| | GUUGGUGGCCCACGCCGGCCAACUCACCGCAGUCUGGCGCCGCCCAGG |
| | AGCCGCCCGCGCUCCCUGAACCCUAGAACUGUCUUCGACUCAGGGGCCC |
| | CGUUGGAAGACUGAGUGCCCGGGGCACGGCACAGAAGCCGCGCCCACC |
| | GCCUGCCAGUUCACAACCGCUCCGAGCGUGGGUCUCCGCCCAGCACCAG |
| | UCCUGUGAUCCGGGCCCGCCCCCUAGCGGCCGGGGAGGGAGGGGCCGG |
| | GUCCGCGGCCGGCGAACGGGGCUCGAAGGGUCCUUGUAGCCGGGAAUG |
| | CUGCUGCUGCUGCUGGGGGGAUCACAGACCAUUUCUUUCUUUCGGCCA |
| | GGCUGAGGCCCUGACGUGGAUGGGCAAACUGCAGGCCUGGGAAGGCAG |
| | CAAGCCGGGCCGUCCGUGUUCCAUCCUCCACGCACCCCCACCUAUCGUU |
| | GGUUCGCAAAGUGCAAAGCUUUCUUGUGCAUGACGCCCUGCUCUGGGGA |
| | GCGUCUGGCGCGAUCUCUGCCUGCUUACCCGGGAAAUUUGCUUUUGCCA |
| | AACCCGCUUUUUCGGGGAUCCCGCGCCCCCCUCCUCACUUGCGCUGCUC |
| | UCGGAGCCCCAGCCGGCUCCGCCCGCUUCGGCGGUUUGGAUAUUUAUU |
| | GACCUCGUCCUCCGACUCGCUGACAGGCUACAGGACCCCCAACAACCCC |
| | AAUCCACGUUUUGGAUGCACUGAGACCCCGACAUUCCUCGGUAUUUAUU |
| | GUCUGUCCCCACCUAGGACCCCCACCCCCGACCCUCGCGAAUAAAAGGC |
| | CCUCCAUCUGCCCAAAAAAAAAAAAAAAA |
| 2 | GGGACAGCCAGGGACAGGCAGACAUGCAGCCAGGGCUCCAGGGCCUGG |
| | ACAGGGGCUGCCAGGCCCUGUGACAGGAGGACCCCGAGCCCCCGGCCC |
| | GGGGAGGGGCCAUGGUGCUGCCUGUCCAACAUGUCAGCCGAGGUGCGG |
| | CUGAGGCGGCUCCAGCAGCUGGUGUUGGACCCGGGCUUCCUGGGGCUG |
| | GAGCCCCUGCUCGACCUUCUCCUGGGCGUCCACCAGGAGCUGGGCGCC |
| | UCCGAACUGGCCCAGGACAAGUACGUGGCCGACUUCUUGCAGUGGGCG |
| | GAGCCCAUCGUGGUGAGGCUUAAGGAGGUCCGACUGCAGAGGGACGAC |
| | UUCGAGAUUCUGAAGGUGAUCGGACGCGGGGCGUUCAGCGAGGUAGCG |
| | GUAGUGAAGAUGAAGCAGACGGGCCAGGUGUAUGCCAUGAAGAUCAUGA |
| | ACAAGUGGGACAUGCUGAAGAGGGGCGAGGUGUCGUGCUUCCGUGAGG |
| | AGAGGGACGUGUUGGUGAAUGGGGACCGGCGGUGGAUCACGCAGCUGC |
| | ACUUCGCCUUCCAGGAUGAGAACUACCUGUACCUGGUCAUGGAGUAUUA |
| | CGUGGGCGGGGACCUGCUGACACUGCUGAGCAAGUUUGGGGAGCGGAU |
| | UCCGGCCGAGAUGGCGCGCUUCUACCUGGCGGAGAUUGUCAUGGCCAU |
| | AGACUCGGUGCACCGGCUUGGCUACGUGCACAGGGACAUCAAACCCGAC |
| | AACAUCCUGCUGGACCGCUGUGGCCACAUCCGCCUGGCCGACUUCGGCU |
| | CUUGCCUCAAGCUGCGGGCAGAUGGAACGGUGCGGUCGCUGGUGGCUG |
| | UGGGCACCCCAGACUACCUGUCCCCCGAGAUCCUGCAGGCUGUGGGCG |
| | GUGGGCCUGGGACAGGCAGCUACGGGCCCGAGUGUGACUGGUGGGCGC |
| | UGGGUGUAUUCGCCUAUGAAAUGUUCUAUGGGCAGACGCCCUUCUACGC |
| | GGAUUCCACGGCGGAGACCUAUGGCAAGAUCGUCCACUACAAGGAGCAC |
| | CUCUCUCUGCCGCUGGUGGACGAAGGGGUCCCUGAGGAGGCUCGAGAC |
| | UUCAUUCAGCGGUUGCUGUGUCCCCCGGAGACACGGCUGGGCCGGGGU |
| | GGAGCAGGCGACUUCCGGACACAUCCCUUCUUCUUUGGCCUCGACUGGG |
| | AUGGUCUCCGGGACAGCGUGCCCCCCUUUACACCGGAUUUCGAAGGUGC |
| | CACCGACACAUGCAACUUCGACUUGGUGGAGGACGGGCUCACUGCCAUG |
| | GUGAGCGGGGGCGGGGAGACACUGUCGGACAUUCGGGAAGGUGCGCCG |
| | CUAGGGGUCCACCUGCCUUUUGUGGGCUACUCCUACUCCUGCAUGGCCC |
| | UCAGGGACAGUGAGGUCCCAGGCCCCACACCCAUGGAACUGGAGGCCGA |
| | GCAGCUGCUUGAGCCACACGUGCAAGCGCCCAGCCUGGAGCCCUCGGU |
| | GUCCCCACAGGAUGAAACAGCUGAAGUGGCAGUUCCAGCGGCUGUCCCU |
| | GCGGCAGAGGCUGAGGCCGAGGUGACGCUGCGGGAGCUCCAGGAAGCC |
| | CUGGAGGAGGAGGUGCUCACCCGGCAGAGCCUGAGCCGGGAGAUGGAG |
| | GCCAUCCGCACGGACAACCAGAACUUCGCCAGUCAACUACGCGAGGCAG |
| | AGGCUCGGAACCGGGACCUAGAGGCACACGUCCGGCAGUUGCAGGAGC |
| | GGAUGGAGUUGCUGCAGGCAGAGGGAGCCACAGCUGUCACGGGGGUCC |
| | CCAGUCCCCGGGCCACGGAUCCACCUUCCCAUCUAGAUGGCCCCCCGGC |
| | CGUGGCUGUGGGCCAGUGCCCGCUGGUGGGGCCAGGCCCCAUGCACCG |
| | CCGCCACCUGCUGCUCCCUGCCAGGGUCCCUAGGCCUGGCCUAUCGGA |
| | GGCGCUUUCCCUGCUCCUGUUCGCCGUUGUUCUGUCUCGUGCCGCCGC |
| | CCUGGGCUGCAUUGGGUUGGUGGCCCACGCCGGCCAACUCACCGCAGU |
| | CUGGCGCCGCCCAGGAGCCGCCCGCGCUCCCUGAACCCUAGAACUGUCU |
| | UCGACUCCGGGGCCCCGUUGGAAGACUGAGUGCCCGGGGCACGGCACA |
| | GAAGCCGCGCCCACCGCCUGCCAGUUCACAACCGCUCCGAGCGUGGGUC |
| | UCCGCCCAGCUCCAGUCCUGUGAUCCGGGCCCGCCCCCUAGCGGCCGG |
| | GGAGGGAGGGGCCGGGUCCGCGGCCGGCGAACGGGGCUCGAAGGGUCC |
| | UUGUAGCCGGGAAUGCUGCUGCUGCUGCUGGGGGGAUCACAGACCAUU |
| | UCUUUCUUUCGGCCAGGCUGAGGCCCUGACGUGGAUGGGCAAACUGCA |
| | GGCCUGGGAAGGCAGCAAGCCGGGCCGUCCGUGUUCCAUCCUCCACGC |
| | ACCCCCACCUAUCGUUGGUUCGCAAAGUGCAAAGCUUUCUUGUGCAUGA |
| | CGCCCUGCUCUGGGGAGCGUCUGGCGCGAUCUCUGCCUGCUUACUCGG |
| | GAAAUUUGCUUUUGCCAAACCCGCUUUUUCGGGGAUCCCGCGCCCCCCU |
| | CCUCACUUGCGCUGCUCUCGGAGCCCCAGCCGGCUCCGCCCGCUUCGG |
| | CGGUUUGGAUAUUUAUUGACCUCGUCCUCCGACUCGCUGACAGGCUACA |
| | GGACCCCCAACAACCCCAAUCCACGUUUUGGAUGCACUGAGACCCCGAC |
| | AUUCCUCGGUAUUUAUUGUCUGUCCCCACCUAGGACCCCCACCCCCGAC |
| | CCUCGCGAAUAAAAGGCCCUCCAUCUGCCCAAAAAAAAAAAAAAAAAAAAA |
| | AAAAAAAA |

The terms “dystrophia myotonica protein kinase” and “DMPK” as used herein include, for example, forms of the human DMPK gene that have a nucleic acid sequence that is at least 85% identical to the nucleic acid sequence of SEQ ID NO: 1 or SEQ ID NO: 2 (e.g., 85%, 86%, 87%, 88%, 89%, 90%, 91%, 92%, 93%, 94%, 95%, 96%, 97%, 98%, 99%, 99.9%, or 100% identical to the nucleic acid sequence of SEQ ID NO: 1 or SEQ ID NO: 2) and/or forms of the human DMPK gene that encode a DMPK protein having one or more (e.g., up to 25) conservative amino acid substitutions relative to a wild-type DMPK protein. The terms “dystrophia myotonica protein kinase” and “DMPK” as used herein additionally include DMPK RNA transcripts containing expanded CUG trinucleotide repeat regions relative to the length of the CUG trinucleotide repeat region of a wild-type DMPK mRNA transcript. The expanded repeat region may contain, for example, 50 or more CUG trinucleotide repeats, such as from about 50 to about 4,000 CUG trinucleotide repeats (e.g., about 50 CUG trinucleotide repeats, about 60 CUG trinucleotide repeats, about 70 trinucleotide repeats, 80 trinucleotide repeats, 90 trinucleotide repeats, 100 trinucleotide repeats, 110 trinucleotide repeats, 120 trinucleotide repeats, 130 trinucleotide repeats, 140 trinucleotide repeats, 150 trinucleotide repeats, 160 trinucleotide repeats, 170 trinucleotide repeats, 180 trinucleotide repeats, 190 trinucleotide repeats, 200 trinucleotide repeats, 210 trinucleotide repeats, 220 trinucleotide repeats, 230 trinucleotide repeats, 240 trinucleotide repeats, 250 trinucleotide repeats, 260 trinucleotide repeats, 270 trinucleotide repeats, 280 trinucleotide repeats, 290 trinucleotide repeats, 300 trinucleotide repeats, 310 trinucleotide repeats, 320 trinucleotide repeats, 330 trinucleotide repeats, 340 trinucleotide repeats, 350 trinucleotide repeats, 360 trinucleotide repeats, 370 trinucleotide repeats, 380 trinucleotide repeats, 390 trinucleotide repeats, 400 trinucleotide repeats, 410 trinucleotide repeats, 420 trinucleotide repeats, 430 trinucleotide repeats, 440 trinucleotide repeats, 450 trinucleotide repeats, 460 trinucleotide repeats, 470 trinucleotide repeats, 480 trinucleotide repeats, 490 trinucleotide repeats, 500 trinucleotide repeats, 510 trinucleotide repeats, 520 trinucleotide repeats, 530 trinucleotide repeats, 540 trinucleotide repeats, 550 trinucleotide repeats, 560 trinucleotide repeats, 570 trinucleotide repeats, 580 trinucleotide repeats, 590 trinucleotide repeats, 600 trinucleotide repeats, 610 trinucleotide repeats, 620 trinucleotide repeats, 630 trinucleotide repeats, 640 trinucleotide repeats, 650 trinucleotide repeats, 660 trinucleotide repeats, 670 trinucleotide repeats, 680 trinucleotide repeats, 690 trinucleotide repeats, 700 trinucleotide repeats, 710 trinucleotide repeats, 720 trinucleotide repeats, 730 trinucleotide repeats, 740 trinucleotide repeats, 750 trinucleotide repeats, 760 trinucleotide repeats, 770 trinucleotide repeats, 780 trinucleotide repeats, 790 trinucleotide repeats, 800 trinucleotide repeats, 810 trinucleotide repeats, 820 trinucleotide repeats, 830 trinucleotide repeats, 840 trinucleotide repeats, 850 trinucleotide repeats, 860 trinucleotide repeats, 870 trinucleotide repeats, 880 trinucleotide repeats, 890 trinucleotide repeats, 900 trinucleotide repeats, 910 trinucleotide repeats, 920 trinucleotide repeats, 930 trinucleotide repeats, 940 trinucleotide repeats, 950 trinucleotide repeats, 960 trinucleotide repeats, 970 trinucleotide repeats, 980 trinucleotide repeats, 990 trinucleotide repeats, 1,000 trinucleotide repeats, 1,100 trinucleotide repeats, 1,200 trinucleotide repeats, 1,300 trinucleotide repeats, 1,400 trinucleotide repeats, 1,500 trinucleotide repeats, 1,600 trinucleotide repeats, 1,700 trinucleotide repeats, 1,800 trinucleotide repeats, 1,900 trinucleotide repeats, 2,000 trinucleotide repeats, 2,100 trinucleotide repeats, 2,200 trinucleotide repeats, 2,300 trinucleotide repeats, 2,400 trinucleotide repeats, 2,500 trinucleotide repeats, 2,600 trinucleotide repeats, 2,700 trinucleotide repeats, 2,800 trinucleotide repeats, 2,900 trinucleotide repeats, 3,000 trinucleotide repeats, 3,100 trinucleotide repeats, 3,200 trinucleotide repeats, 3,300 trinucleotide repeats, 3,400 trinucleotide repeats, 3,500 trinucleotide repeats, 3,600 trinucleotide repeats, 3,700 trinucleotide repeats, 3,800 trinucleotide repeats, 3,900 trinucleotide repeats, or 4,000 trinucleotide repeats, among others).

As used herein, the term “interfering RNA” refers to a RNA, such as a short interfering RNA (siRNA), micro RNA (miRNA), or short hairpin RNA (shRNA) that suppresses the expression of a target RNA transcript by way of (i) annealing to the target RNA transcript, thereby forming a nucleic acid duplex; and (ii) promoting the nuclease-mediated degradation of the RNA transcript and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient, such as a human patient having myotonic dystrophy, in the form of, for example, a single- or double-stranded oligonucleotide, or in the form of a vector (e.g., a viral vector, such as an adeno-associated viral vector described herein) containing a transgene encoding the interfering RNA. Exemplary interfering RNA platforms are described, for example, in Lam et al., Molecular Therapy-Nucleic Acids 4:e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

As used herein, the “length” of a nucleic acid refers to the linear size of the nucleic acid as assessed by measuring the quantity of nucleotides from the 5′ to the 3′ end of the nucleic acid. Exemplary molecular biology techniques that may be used to determine the length of a nucleic acid of interest are known in the art.

As used herein, the term “myotonic dystrophy” refers to an inherited muscle wasting disorder characterized by the nuclear retention of RNA transcripts encoding DMPK and containing an expanded CUG trinucleotide repeat region in the 3′ untranslated region (UTR), such as an expanded CUG trinucleotide repeat region having from 50 to 4,000 CUG repeats. Wild-type RMPK RNA transcripts, by comparison, typically contain from 5 to 37 CUG repeats in the 3′ UTR. In patients having myotonic dystrophy, the expanded CUG repeat region interacts with RNA-binding splicing factors, such as muscleblind-like protein. This interaction causes the mutant transcript to be retained in nuclear foci and leads to sequestration of RNA-binding proteins away from other pre-mRNA substrates, which, in turn, promotes spliceopathy of proteins involved in modulating muscle structure and function. In type I myotonic dystrophy (DM1), skeletal muscle is often the most severely affected tissue, but the disease also imparts toxic effects on cardiac and smooth muscle, the ocular lens, and the brain. The cranial, distal limb, and diaphragm muscles are preferentially affected. Manual dexterity is compromised early, which causes several decades of severe disability. The median age at death of myotonic dystrophy patients is 55 years, which is usually caused by respiratory failure (de Die-Smulders C E, et al., Brain 121:1557-1563 (1998)).

As used herein, the term “operably linked” refers to a first molecule (e.g., a first nucleic acid) joined to a second molecule (e.g., a second nucleic acid), wherein the molecules are so arranged that the first molecule affects the function of the second molecule. The two molecules may or may not be part of a single contiguous molecule and may or may not be adjacent to one another. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, one segment of a nucleic acid molecule is considered to “overlap with” another segment of the same nucleic acid molecule if the two segments share one or more constituent nucleotides. For example, two segments of the same nucleic acid molecule are considered to “overlap with” one another if the two segments share 1, 2, 3, 4, 5, 6, 7, 8, 9, 10, 15, 20, 25, 30, 35, 40, 45, 50, 100, or more, constituent nucleotides. The two segments are not considered to “overlap with” one another if the two segments have zero constituent nucleotides in common.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, such as a nucleic acid or vector described herein, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions, and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “repeat region” refers to segments within a gene of interest or an RNA transcript thereof containing nucleic acid repeats, such as the poly CTG sequence in the 3″ UTR of the human DMPK gene (or the poly CUG sequence in the 3′ UTR of the RNA transcript thereof). A repeat region is considered to be an “expanded repeat region,” a “repeat expansion,” or the like, if the number of nucleotide repeats in the repeat region exceeds the quantity of repeats ordinarily found in the repeat region of a wild-type form of the gene or RNA transcript thereof. For example, the 3′ UTRs of wild-type human DMPK genes typically contain from 5 to 37 CTG or CUG repeats. “Expanded repeat regions” and “repeat expansions” in the context of the DMPK gene or an RNA transcript thereof thus refer to repeat regions containing greater than 37 CTG or CUG repeats, such as from about 50 to about 4,000 CUG trinucleotide repeats (e.g., about 50 CUG trinucleotide repeats, about 60 CUG trinucleotide repeats, about 70 trinucleotide repeats, 80 trinucleotide repeats, 90 trinucleotide repeats, 100 trinucleotide repeats, 110 trinucleotide repeats, 120 trinucleotide repeats, 130 trinucleotide repeats, 140 trinucleotide repeats, 150 trinucleotide repeats, 160 trinucleotide repeats, 170 trinucleotide repeats, 180 trinucleotide repeats, 190 trinucleotide repeats, 200 trinucleotide repeats, 210 trinucleotide repeats, 220 trinucleotide repeats, 230 trinucleotide repeats, 240 trinucleotide repeats, 250 trinucleotide repeats, 260 trinucleotide repeats, 270 trinucleotide repeats, 280 trinucleotide repeats, 290 trinucleotide repeats, 300 trinucleotide repeats, 310 trinucleotide repeats, 320 trinucleotide repeats, 330 trinucleotide repeats, 340 trinucleotide repeats, 350 trinucleotide repeats, 360 trinucleotide repeats, 370 trinucleotide repeats, 380 trinucleotide repeats, 390 trinucleotide repeats, 400 trinucleotide repeats, 410 trinucleotide repeats, 420 trinucleotide repeats, 430 trinucleotide repeats, 440 trinucleotide repeats, 450 trinucleotide repeats, 460 trinucleotide repeats, 470 trinucleotide repeats, 480 trinucleotide repeats, 490 trinucleotide repeats, 500 trinucleotide repeats, 510 trinucleotide repeats, 520 trinucleotide repeats, 530 trinucleotide repeats, 540 trinucleotide repeats, 550 trinucleotide repeats, 560 trinucleotide repeats, 570 trinucleotide repeats, 580 trinucleotide repeats, 590 trinucleotide repeats, 600 trinucleotide repeats, 610 trinucleotide repeats, 620 trinucleotide repeats, 630 trinucleotide repeats, 640 trinucleotide repeats, 650 trinucleotide repeats, 660 trinucleotide repeats, 670 trinucleotide repeats, 680 trinucleotide repeats, 690 trinucleotide repeats, 700 trinucleotide repeats, 710 trinucleotide repeats, 720 trinucleotide repeats, 730 trinucleotide repeats, 740 trinucleotide repeats, 750 trinucleotide repeats, 760 trinucleotide repeats, 770 trinucleotide repeats, 780 trinucleotide repeats, 790 trinucleotide repeats, 800 trinucleotide repeats, 810 trinucleotide repeats, 820 trinucleotide repeats, 830 trinucleotide repeats, 840 trinucleotide repeats, 850 trinucleotide repeats, 860 trinucleotide repeats, 870 trinucleotide repeats, 880 trinucleotide repeats, 890 trinucleotide repeats, 900 trinucleotide repeats, 910 trinucleotide repeats, 920 trinucleotide repeats, 930 trinucleotide repeats, 940 trinucleotide repeats, 950 trinucleotide repeats, 960 trinucleotide repeats, 970 trinucleotide repeats, 980 trinucleotide repeats, 990 trinucleotide repeats, 1,000 trinucleotide repeats, 1,100 trinucleotide repeats, 1,200 trinucleotide repeats, 1,300 trinucleotide repeats, 1,400 trinucleotide repeats, 1,500 trinucleotide repeats, 1,600 trinucleotide repeats, 1,700 trinucleotide repeats, 1,800 trinucleotide repeats, 1,900 trinucleotide repeats, 2,000 trinucleotide repeats, 2,100 trinucleotide repeats, 2,200 trinucleotide repeats, 2,300 trinucleotide repeats, 2,400 trinucleotide repeats, 2,500 trinucleotide repeats, 2,600 trinucleotide repeats, 2,700 trinucleotide repeats, 2,800 trinucleotide repeats, 2,900 trinucleotide repeats, 3,000 trinucleotide repeats, 3,100 trinucleotide repeats, 3,200 trinucleotide repeats, 3,300 trinucleotide repeats, 3,400 trinucleotide repeats, 3,500 trinucleotide repeats, 3,600 trinucleotide repeats, 3,700 trinucleotide repeats, 3,800 trinucleotide repeats 3,900 trinucleotide repeats, or 4,000 trinucleotide repeats, among others).

As used herein, the term “RNA dominance” refers to a pathology that is induced by the expression and nuclear retention of RNA transcripts containing expanded repeat regions relative to the quantity of repeat regions, if any, contained by a wild-type form of the RNA transcript of interest. The toxic effects of RNA dominance are a manifestation, for example, of the binding interaction between the expanded repeat regions of the pathologic, mutant RNA transcripts with splicing factor proteins, which promotes the sequestration of splicing factors away from pre-mRNA transcripts, thereby engendering spliceopathy among such substrates. Exemplary disorders associated with RNA dominance are myotonic dystrophy and amyotrophic lateral sclerosis, as described herein, among others.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, or cells) isolated from a subject. The subject may be, for example, a patient suffering from a disease described herein, such as a heritable muscle wasting disorder (e.g., muscular dystrophy, such as myotonic dystrophy (e.g., myotonic dystrophy type I).

As used herein, the phrases “specifically binds” and “binds” refer to a binding reaction which is determinative of the presence of a particular molecule, such as an RNA transcript, in a heterogeneous population of ions, salts, small molecules, and/or proteins that is recognized, e.g., by a ligand or receptor, such as an RNA-binding splicing factor protein, with particularity. A ligand (e.g., an RNA-binding protein described herein) that specifically binds to a species (e.g., an RNA transcript) may bind to the species, e.g., with a K_{D} of less than 1 mM. For example, a ligand that specifically binds to a species may bind to the species with a K_{D} of up to 100 μM (e.g., between 1 μM and 100 μM). A ligand that does not exhibit specific binding to another molecule may exhibit a K_{D} of greater than 1 mM (e.g., 1 μM, 100 μM, 500 μM, 1 mM, or greater) for that particular molecule or ion. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the term “spliceopathy” refers to a change in the splicing pattern of an mRNA transcript that leads to the expression of one or more alternative splice products relative to a wild-type form of the mRNA transcript of interest. Spliceopathy can lead to a toxic loss of function if, for example, the mRNA transcript is spliced in such a way that one or more exons necessary for the activity of the encoded protein are no longer present in the mRNA transcript upon translation. Additionally or alternatively, toxic loss of function may occur due to the aberrant inclusion of one or more introns, for example, in a manner that precludes the proper folding of the encoded protein.

As used herein, the terms “subject” and “patient” refer to an organism that receives treatment for a particular disease or condition as described herein (such as a heritable muscle-wasting disorder, e.g., myotonic dystrophy). Examples of subjects and patients include mammals, such as humans, receiving treatment for a disease or condition described herein.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Goeddel, Gene Expression Technology: Methods in Enzymology 185 (Academic Press, San Diego, Calif., 1990).

As used herein, the terms “treat” or “treatment” refer to therapeutic treatment, in which the object is to prevent or slow down (lessen) an undesired physiological change or disorder, such as the progression of a heritable muscle-wasting disorder, for example, myotonic dystrophy, and particularly, type I myotonic dystrophy. In the context of myotonic dystrophy treatment, beneficial or desired clinical results that are indicative of successful treatment include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment of a patient having myotonic dystrophy (e.g., type I myotonic dystrophy may manifest in one or more detectable changes, such as a decrease in the expression of DMPK RNA transcripts that contain expanded CUG trinucleotide repeat regions (e.g., a decrease in the expression of DMPK RNA transcripts that contain expanded CUG trinucleotide repeat regions of 1% or more, such as a decrease of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%, relative to the expression of DMPK RNA transcripts containing expanded CUG trinucleotide repeat regions by the patient prior to administration of a therapeutic agent, such as a vector or nucleic acid described herein. Methods that can be used to assess RNA expression levels are known in the art and include RNA-seq assays and polymerase chain reaction techniques described herein. Additional clinical indications of successful treatment of a CPVT patient include alleviation of spliceopathy, for example, of an RNA transcript that is spliced in a manner that is dependent upon muscleblind-like protein. For example, observations that signal successful treatment of a patient having myotonic dystrophy include a finding that the patient exhibits an increase in corrective splicing of one or more RNA transcript substrates of muscleblind-like protein following administration of a therapeutic agent, such as a therapeutic agent described herein. For example, indicators that signal successful treatment of myotonic dystrophy include a determination that the patient exhibits an increase in expression of sarcoplasmic/endoplasmic reticulum calcium ATPase 1 (SERCA1) mRNA containing exon 22, such as an increase of about 1.1-fold to about 10-fold, or more (e.g., an increase in expression of SERCA1 mRNA containing exon 22 by about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, or more), as assessed, for example, using an RNA or protein detection assay described herein. Treatment of myotonic dystrophy may also manifest as a decrease in expression of chloride voltage-gated channel 1 (CLCN1) mRNA containing exon 7a, such as a decrease of about 1% to about 100% (e.g., a decrease in expression of CLCN1 mRNA containing exon 7a by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%), as assessed, for example, using an RNA or protein detection assay described herein. Additionally, successful treatment may be signaled by a determination that the patient exhibits a decrease in expression of ZO-2 associated speckle protein (ZASP) containing exon 11, such as a decrease of about 1% to about 100% (e.g., a decrease in expression of ZASP mRNA containing exon 11 by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%), as assessed, for example, using an RNA or protein detection assay described herein. Successful treatment of myotonic dystrophy may also be signaled by a finding that, following the therapy, the patient exhibits an increase in corrective splicing of RNA transcripts encoding insulin receptor, ryanodine receptor 1 (RYR1), cardiac muscle troponin, and/or skeletal muscle troponin, such as an increase of about 1.1-fold to about 10-fold, or more (e.g., an increase in expression of correctly spliced RNA transcripts encoding insulin receptor, RYR1, cardiac muscle troponin, and/or skeletal muscle troponin by about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, or more), as assessed, for example, using an RNA or protein detection assay described herein. Additional clinical indications of successful treatment of myotonic dystrophy include improvements in muscle function, such as in the cranial, distal limb, and diaphragm muscles.

As used herein, the term “vector” refers to a nucleic acid, e.g., DNA or RNA, that may function as a vehicle for the delivery of a gene of interest into a cell (e.g., a mammalian cell, such as a human cell), tissue, organ, or organism, such as a patient undergoing treatment for a disease or condition described herein, for purposes of expressing an encoded transgene. Exemplary vectors useful in conjunction with the compositions and methods described herein are plasmids, DNA vectors, RNA vectors, virions, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### BRIEF DESCRIPTION OF THE FIGURES

FIG. 1 is a diagram showing the structure of human dystrophia myotonica protein kinase (DMPK) RNA, including the configuration of exons (represented by shaded rectangular boxes) and the site of the CUG trinucleotide repeat region. Numerical values from 600 to 12,600 along the bottom of the figure indicate nucleotide position along the length of the DMPK RNA transcript. The diagram shows the regions within the DMPK RNA transcript to which various exemplary interfering RNA constructs described herein anneal by way of sequence complementarity.

FIGS. 2A-2C are diagrams showing the schematics of three generations of rAAV vectors encoding interfering RNA molecules (rAAV-RNAi vectors) targeting several genes of interest, such as those associated with RNA dominance. The rAAV plasmid pARAP4 includes the human alkaline phosphatase reporter gene (Hu Alk Phos) reporter gene, expressed from the Rous Sarcoma Virus (RSV) promoter, and the SV40 polyadenylation sequence, pA. The inverted terminal repeats (ITRs) originate from rAAV2, and the genomes are packaged in rAAV6 capsids. The newest vector modification removes the RSV promoter sequence to prevent Hu Alk Phos expression that limited rAAV-RNAi efficacy at higher doses due muscle cell toxicity.

FIG. 3A is a diagram showing exemplary routes of administration of rAAV-RNAi vectors into murine models of RNA dominance disorders, such as myotonic dystrophy.

FIGS. 3B and 3C are diagrams comparing various characteristics of myotonic dystrophy type I (DM1) and the murine HSA^{LR} model of this disease. HSA^{LR} mice show characteristics of myotonic dystrophy resembling DM in humans. The HSA^{LR} transgene is derived from insertion of a (CTG)₂₅₀ repeat in the 3′ UTR of the human skeletal actin (HSA) gene. When the transgene is expressed in mouse skeletal muscle, myotonic discharges are evident, splicing alterations occur in a variety of mRNAs, and nuclear foci containing the expanded transgenic mRNA and splicing factors are present.

FIG. 4 is a diagram showing the characteristics of HSA^{LR} mice transduced with rAAV6 HSA miR DM10, as described in Example 1, below. Human placental alkaline phosphatase (AP) staining indicates presence of the viral genome with active reporter gene expression. H&E staining of cryosections from treated mice. Timepoint, 8 weeks post-injection, 4 week-old HSA^{LR} mice.

FIGS. 5A and 5B are graphs quantifying HSA mRNA and expression of the HSA miRDM10 in seven individual HSA^{LR} mice transduced as described in Example 1, below. mRNA expression shown was assessed by qPCR at 8 weeks post-rAAV injection.

FIGS. 6A-6E are diagrams demonstrating that rAAV6 HSA miR DM10 systemic injection improves splicing of Atp2a (SERCA1) and CLCN1 in the tibialis anterior (TA) muscle, as described in Example 1, below. In contrast, a different RNAi hairpin, miR DM4, is not as effective at reversing these splicing defects.

FIGS. 7A-7C are diagrams showing the structure and activity of re-engineered rAAV-miR DM10 and -miR DM4 evaluated in vivo. Intramuscular injection of vectors without the RSV promoter to prevent Hu Alk Phos expression to assess the efficacy of gene silencing compared to the previously tested Alk Phos expressing vectors. FIG. 7A shows a schematic diagram of the rAAV genome lacking the RSV promoter sequence as the ‘new DM10’ and ‘new DM4’ labeled on the gels in FIG. 7B. FIG. 7B shows analysis of splicing patterns for Atp2a1/Serca1 following IM injection of ‘new DM10’ and ‘new DM4’ into the TA muscle compared to Alk Phos expressing ‘old DM10.’ L=low dose of 5×10⁹ vector genomes; H=high dose of 5×10¹⁰ vector genomes. −, no RSV promoter; +, RSV present in vector genome. The high dose was chosen because it elicited some muscle regeneration as marked by the presence of central nuclei (CN) with IM injection of Hu Alk Phos expressing vectors. However, no evidence of muscle turnover was observed at this high dose of rAAV DM10 lacking RSV, new DM10 and new DM4.

FIG. 8A is a diagram showing how purified plasmids expressing the DMPK-targeting miRNAs were transfected into HEK293 cells and RNA was isolated and subjected to RT-qPCR to evaluate DMPK transcript engagement by the RISC complex and Dicer cleavage.

FIG. 8B is a graph showing the evaluation of the gene silencing activity of U6 DMPK miRNAs. Candidate therapeutic miRNA expression cassettes a and b showed reduction of the endogenous DMPK mRNA 48 hrs after transfection of HEK293 cells with 1.5 μg of plasmid DNA compared to a plasmid with no miRNA expression cassette. Eight biological replicates were assayed per plasmid and control. Along the x-axis, “a” represents a miRNA containing a passenger strand having the nucleic acid sequence of SEQ ID NO: 42 and a guide strand having the nucleic acid sequence of SEQ ID NO: 79; “b” represents a miRNA containing a passenger strand having the nucleic acid sequence of SEQ ID NO: 44 and a guide strand having the nucleic acid sequence of SEQ ID NO: 81; “c” represents a miRNA containing a passenger strand having the nucleic acid sequence of SEQ ID NO: 46 and a guide strand having the nucleic acid sequence of SEQ ID NO: 83; “d” represents a miRNA containing a passenger strand having the nucleic acid sequence of SEQ ID NO: 47 and a guide strand having the nucleic acid sequence of SEQ ID NO: 84; and “none” represents cells not treated with an anti-DMPK miRNA.

FIG. 9 is a graph showing the ability of various siRNA molecules described herein to downregulate DMPK expression in HEK293 cells, as described in Example 4, below. Values along the y-axis represent normalized DMPK expression, and the x-axis shows the anti-DMPK siRNA molecules tested. The first entry on the x-axis corresponds to treatment of HEK293 cells with transfection vehicle only, and the second entry on the x-axis represents treatment with an siRNA having a scrambled nucleic acid sequence as a negative control. siRNA molecules having a specified sequence identifier number are described herein, for example, in Table 4, below. siRNA molecules labeled “Anti-DMPK” followed by an alphanumeric identifier are commercially available from Thermo Fisher Scientific.

### DETAILED DESCRIPTION

The compositions and methods described herein are useful for reducing the occurrence of spliceopathy and for treating disorders associated with ribonucleic acid (RNA) dominance, such as myotonic dystrophy and amyotrophic lateral sclerosis, among others. The compositions described herein include nucleic acids containing interfering RNA constructs that suppress the expression of RNA transcripts containing aberrantly expanded repeat regions. This activity provides an important physiological benefit, as various RNA transcripts harboring such repeat expansions exhibit a heightened avidity for RNA splicing factors. This avidity manifests in sequestration of RNA splicing factors away from other pre-mRNA substrates, thereby disrupting the proper splicing of these transcripts. Without being limited by mechanism, the compositions described herein may ameliorate this pathology by diminishing the expression of RNA transcripts harboring expanded nucleotide repeats, thus releasing sequestered splicing factors so that they may properly regulate the splicing of various other pre-mRNA transcripts. For example, the compositions and methods described herein may be used to treat disorders, such as myotonic dystrophy, associated with expression of dystrophia myotonica protein kinase (DMPK) RNA transcripts containing an expanded CUG trinucleotide repeat region. Similarly, the compositions and methods described herein may be used to treat amyotrophic lateral sclerosis, characterized by elevated expression of C9ORF72 RNA transcripts containing GGGGCC (SEQ ID NO: 162) hexanucleotide repeats.

The interfering RNA constructs described herein may be in any of a variety of forms, such as short interfering RNA (siRNA), short hairpin RNA (shRNA), or micro RNA (miRNA). The interfering RNAs described herein may additionally be encoded by a vector, such as a viral vector. For example, described herein are adeno-associated viral (AAV) vectors, such as pseudotyped AAV vectors (e.g., AAV2/8 and AAV2/9 vectors) containing transgenes encoding interfering RNA constructs that attenuate the expression of RNA transcripts harboring expanded nucleotide repeats.

The compositions and methods described herein provide, among other benefits, the advantageous feature of being able to selectively suppress the expression of pathologic RNA transcripts among other RNAs that contain expanded nucleotide repeat regions. This property is particularly beneficial in view of the prevalence of nucleotide repeats in mammalian genomes, such as in the genomes of human patients. Using the compositions and methods described herein, the expression of RNA transcripts that contain pathological nucleotide repeat expansions can be diminished, while preserving the expression of important healthy RNA transcripts and their encoded protein products.

This advantageous feature is based, in part, on the surprising discovery that interfering RNA constructs that anneal to repeat-expanded RNA targets at sites remote from the expanded repeat region can be used to suppress the expression of these RNA transcripts and release splicing factor proteins that would otherwise be sequestered by these molecules. The compositions and methods described herein can thus attenuate the expression and nuclear retention of pathological RNA transcripts without the necessity of containing complementary nucleotide repeat motifs.

The sections that follow provide a description of exemplary interfering RNA constructs that may be used in conjunction with the compositions and methods described herein, as well as a description of vectors encoding such constructs and procedures that may be used to treat disorders associated with spliceopathy, such as myotonic dystrophy and amyotrophic lateral sclerosis.

### Methods of Treating RNA Dominance and Correcting Spliceopathy

Using the compositions and methods described herein, a patient experiencing a spliceopathy and/or having a disease associated with RNA dominance, such as myotonic dystrophy, among others, can be administered a nucleic acid containing an interfering RNA construct, or a vector encoding the same, so as to reduce the expression of RNA transcripts containing expanded repeat regions. Without being limited by mechanism, this activity provides the beneficial effect of releasing RNA-binding proteins that bind with high avidity to the repeat expansion regions of pathologic RNA transcripts. The release of such RNA-binding proteins is important, as the proteins sequestered by binding to repeat-expanded RNA transcripts include splicing factors that would ordinarily be available to modulate the proper splicing of various pre-mRNA transcripts. In patients having RNA dominance disorders, such as myotonic dystrophy, splicing factors such as muscleblind-like protein, which regulates the splicing of various transcripts that encode proteins having important roles in regulating muscle function, are sequestered from important pre-mRNA substrates. The compositions and methods described herein may treat RNA dominance disorders by promoting the degradation of RNA transcripts containing expanded nucleotide repeat regions, thereby effectuating the release of significant RNA-binding proteins from such transcripts.

### Myotonic Dystrophy Type I

Myotonic dystrophy type I is the most common form of muscular dystrophy in adults, and occurs with an estimated frequency of 1 in 7,500 (Harper P S., Myotonic Dystrophy. London: W.B. Saunders Company; 2001). This disease is an autosomal dominant disorder caused by expansion of a non-coding CTG repeat in the human DMPK1 gene. DMPK1 is a gene encoding a cytosolic serine/threonine kinase (Brook et al., Cell. 68:799-808 (1992)). The expanded CTG repeat is located in the 3′ untranslated region (UTR) of DMPK1. This mutation leads to RNA dominance, a process in which expression of RNA containing an expanded CUG repeat (CUGexp) induces cell dysfunction (Osborne R J and Thornton C A., Human Molecular Genetics. 15:R162-R169 (2006)).

The mutant form of the DMPK mRNA, harboring large CUG repeats, are fully transcribed and polyadenylated, but remain trapped in the nucleus (Davis et al., Proc. Natl. Acad. Sci. U.S.A 94:7388-7393 (1997)). These mutant, nuclear-retained mRNAs are one of the most important pathological features of myotonic dystrophy type I. The DMPK gene normally has from about 5 to about 37 CTG repeats in the 3′ UTR. In myotonic dystrophy type I, this number is significantly expanded, and may be in the range, for example, of from 50 to greater than 4,000 repeats. The CUGexp tract in the ensuing RNA transcript interacts with RNA-binding splicing factor proteins, including muscleblind-like protein. The enhanced avidity engendered by the expanded CUG repeat region causes the mutant transcript to retain such splicing factor proteins in nuclear foci. The toxicity of this mutant RNA stems from the sequestration of RNA-binding splicing factor proteins away from other pre-mRNA substrates, including those that encode proteins that have important roles in regulating muscle function.

In myotonic dystrophy type I, skeletal muscle is the most severely affected tissue, but the disease also has important effects on cardiac and smooth muscle, ocular lens, and the brain. Among muscle tissue, the cranial, distal limb, and diaphragm muscles are often preferentially affected. Manual dexterity is compromised early, which causes several decades of severe disability. The median age at death is 55 years, which usually from respiratory failure (de Die-Smulders C E, et al., Brain 121(Pt 8):1557-1563 (1998)). Symptoms of myotonic dystrophy include, without limitation, myotonia, muscle stiffness, disabling distal weakness, weakness in the face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. In children, symptoms may also include developmental delays, learning problems, language and speech difficulties, and personality development challenges.

### Pathogenic DMPK Transcripts

Myotonic dystrophy patients that may be treated using the compositions and methods described herein include patients, such as human patient, having myotonic dystrophy type I, and that express a DMPK RNA transcript harboring a CUG repeat expansion. Exemplary DMPK RNA transcripts that may be expressed by a patient undergoing treatment with the compositions and methods described herein are set forth in GenBank Accession Nos. NM_001081560.1, NT_011109.15 (from nucleotides 18540696 to Ser. No. 18/555,106), NT_039413.7 (from nucleotides 16666001 to Ser. No. 16/681,000), NM_032418.1, AI007148.1, AI304033.1, BC024150.1, BC056615.1, BC075715.1, BU519245.1, CB247909.1, CX208906.1, CX732022.1, 560315.1, 560316.1, NM_001081562.1, and NM_001100.3.

### Suppression of Pathologic DMPK RNA Expression

Using the compositions and methods described herein, a patient, such as a patient suffering from myotonic dystrophy (e.g., myotonic dystrophy type I) may be administered a vector encoding, or a composition containing, an interfering RNA that anneals to and suppresses the expression of pathologic DMPK mRNA transcripts. The compositions and methods described herein may selectively attenuate the expression of DMPK mRNA transcripts containing expanded CUG repeats, such as DMPK mRNA transcripts containing from about 50 to about 4,000, or more, CUG repeats. For example, the interfering RNA molecules described herein may activate ribonucleases, such as nuclear ribonucleases, that specifically digest nuclear-retained DMPK transcripts harboring CUG repeat expansions. The decrease in mutant DMPK mRNA expression may be a decrease of, for example, about 1% or more, such as a decrease of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100%, relative to the expression of DMPK mRNA transcripts containing expanded CUG trinucleotide repeat regions by the patient prior to administration of a therapeutic agent described herein, such as a vector or nucleic acid described herein. Methods that can be used to assess RNA expression levels are known in the art and include RNA-seq assays and polymerase chain reaction techniques described herein.

### Correction of Spliceopathy

In some embodiments, the compositions and methods described herein can be used to correct one or more spliceopathies in a patient, such as a patient suffering from myotonic dystrophy (e.g., myotonic dystrophy type I). Without being limited by mechanism, the ability of the interfering RNA molecules described herein to anneal to, and suppresses the expression of, pathologic DMPK transcripts (e.g., DMPK transcripts containing expanded CUG repeat regions) can release splicing factors, such as muscleblind-like protein, that would otherwise be sequestered by way of binding to the CUG repeats. This release of splicing factors may, in turn, effectuate corrective splicing of one or more RNA transcript substrates of these splicing factors. For example, upon administration of the vector or composition to a patient suffering from myotonic dystrophy, the patient may exhibit an increase in expression of sarcoplasmic/endoplasmic reticulum calcium ATPase 1 (SERCA1) mRNA containing exon 22, for example, in the tibialis anterior, gastrocnemius, and/or quadriceps muscles. The increase in expression of SERCA1 mRNA transcripts containing exon 22 may be an increase of, for example, about 1.1-fold to about 10-fold, or more (e.g., an increase in expression of SERCA1 mRNA containing exon 22 by about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, or more), as assessed, for example, using an RNA or protein detection assay described herein.

In some embodiments, upon administration of a vector or composition described herein to a patient suffering from myotonic dystrophy, the patient may exhibit a decrease in expression of chloride voltage-gated channel 1 (CLCN1) mRNA containing exon 7a, for example, in the tibialis anterior, gastrocnemius, and/or quadriceps muscles. The decrease in expression of CLCN1 mRNA transcripts containing exon 7a may be a decrease of, for example, about 1% to about 100% (e.g., a decrease in expression of CLCN1 mRNA containing exon 7a by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%), as assessed, for example, using an RNA or protein detection assay described herein.

Additionally or alternatively, upon administration of a vector or composition described herein to a patient suffering from myotonic dystrophy, the patient may exhibit a decrease in expression of ZO-2 associated speckle protein (ZASP) containing exon 11, for example, in the tibialis anterior, gastrocnemius, and/or quadriceps muscles. The decrease in expression of ZASP mRNA transcripts containing exon 11 may be a decrease of, for example, about 1% to about 100% (e.g., a decrease in expression of ZASP mRNA containing exon 11 by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%), as assessed, for example, using an RNA or protein detection assay described herein.

Additionally or alternatively, upon administration of a vector or composition described herein to a patient suffering from myotonic dystrophy, the patient may exhibit an increase in corrective splicing of RNA transcripts encoding insulin receptor, ryanodine receptor 1 (RYR1), cardiac muscle troponin, and/or skeletal muscle troponin, such as an increase of about 1.1-fold to about 10-fold, or more (e.g., an increase in expression of correctly spliced RNA transcripts encoding insulin receptor, RYR1, cardiac muscle troponin, and/or skeletal muscle troponin by about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, or more), as assessed, for example, using an RNA or protein detection assay described herein.

### Improvement in Muscle Function

The beneficial treatment effects of the compositions and methods described herein, such as the ability of the interfering RNA molecules described herein, and the vectors encoding the same, to (i) suppress pathologic DMPK RNA expression and/or (ii) restore correct splicing of proteins involved in regulating muscle function may manifest clinically in a variety of ways. For example, patients having myotonic dystrophy, such as myotonic dystrophy type I, may exhibit an improvement in cranial, distal limb, and/or diaphragmatic muscle function. The improvement in muscle function may be observed, for example, as an increase in muscle mass, frequency of muscle contractions, and/or magnitude of muscle contractions. For example, using the compositions and methods described herein, a patient suffering from myotonic dystrophy (e.g., myotonic dystrophy type I) may exhibit an increase in cranial, distal limb, and/or diaphragmatic muscle mass, frequency of muscle contractions, and/or magnitude of muscle contractions. The increase in muscle mass, frequency of muscle contractions, and/or magnitude of muscle contractions may be, for example, an increase of 1% or more, such as an increase of from 1% to 25%, from 1% to 50%, from 1% to 75%, from 1% to 100%, from 1% to 500%, from 1% to 1,000%, or more, such as an increase in muscle mass, frequency of muscle contractions, and/or magnitude of muscle contractions of about 1%, 5%, 10%, 15%, 20%, 25%, 50%, 75%, 100%, 200%, 300%, 400%, 500%, 600%, 700%, 80%, 900%, 1,000%, or more.

Particularly, in patients having myotonic dystrophy (e.g., myotonic dystrophy type I), the beneficial therapeutic effects of the interfering RNA molecules described herein, and of the vectors encoding the same, may manifest as a reduction in myotonia. Thus, using the compositions and methods described herein, a patient having myotonic dystrophy (e.g., myotonic dystrophy type I) may be administered an interfering RNA molecule or vector encoding the same so as to facilitate and/or accelerate muscle relaxation. For example, the compositions and methods described herein may be used to accelerate muscle relaxation by suppressing the onset of spontaneous action potentials caused by fluctuations in chloride ion concentration. Without being limited by mechanism, this beneficial activity may be caused by the restoration of correct splicing of CLCN1 mRNA, for example, such that the expression of CLCN1 mRNA containing exon 7a in the patient is reduced. As CLCN1 channel protein regulates chloride ion concentration, correcting the splicing pattern of CLCN1 mRNA transcripts may engender a reduction in the onset of spontaneous action potentials and an improvement in muscle relaxation speed, thereby ameliorating myotonia.

Suppression of myotonia can be evaluated using a variety of techniques known in the art, for example, by way of electromyography. Particularly, electromyography on the left and right quadriceps, left and right gastrocnemius muscles, left and right tibialis anterior muscles, and/or lumbar paraspinals muscles can be performed to assess the effects of the compositions and methods described herein on myotonia in a patient, such as a human patient having myotonic dystrophy. Electromyography protocols have been described, for example, in Kanadia et al., Science 302:1978-1980 (2003)). For example, electromyography may be performed using 30-gauge concentric needle electrodes and a minimum of 10 needle insertions for each muscle. In this way, an average myotonia grade may be determined for a subject, such as a human patient or a model organism (e.g., a murine model of muscular dystrophy described herein). This grade can then be compared to the average myotonia grade of the patient or model organism determined prior to administration of a therapeutic agent described herein (e.g., an interfering RNA molecule or a vector encoding the same). A finding that the average myotonia grade has decreased following administration of the therapeutic agent can serve as an indication of successful treatment of myotonic dystrophy and as an indicator of successful amelioration of the symptom of myotonia.

### Amelioration of Additional Myotonic Dystrophy Symptoms

Using the compositions and methods described herein, a patient having myotonic dystrophy, such as myotonic dystrophy type 1, may be administered an interfering RNA molecule or vector encoding the same so as to attenuate or altogether eliminate one or more symptoms of myotonic dystrophy. Aside from the myotonia described above, symptoms of myotonic dystrophy include, without limitation, muscle stiffness, disabling distal weakness, weakness in the face and jaw muscles, difficulty in swallowing, ptosis, weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. In children, symptoms may also include developmental delays, learning problems, language and speech difficulties, and personality development challenges. The compositions and methods described herein may be used to alleviate one or more, or all, of the foregoing symptoms.

### Duration of Therapeutic Effects

The compositions and methods described herein provide beneficial clinical effects that may last for extended periods of time. For example, using one or more of the interfering RNA molecules described herein, and/or vector(s) encoding the same, a patient having myotonic dystrophy (e.g., myotonic dystrophy type I) may exhibit (i) a reduction in pathologic DMPK RNA expression (e.g., a reduction in expression of DMPK RNA harboring from about 50 to about 4,000 CUG repeats, or more), (ii) an improvement in muscle function (such as an improvement in muscle mass and/or muscle activity, e.g., in the cranial, distal limb, and diaphragm muscle) and/or (iii) alleviation of one or more symptoms of myotonic dystrophy, for a period of one or more days, weeks, months, or years. For example, using the compositions and methods described herein, the beneficial therapeutic effects described herein may be achieved for a period of at least 30 days, at least 35 days, at least 40 days, at least 45 days, at least 50 days, at least 55 days, at least 60 days, at least 65 days, at least 70 days, at least 75 days, at least 76 days, at least 77 days, at least 78 days, at least 79 days, at least 80 days, at least 85 days, at least 90 days, at least 95 days, at least 100 days, at least 105 days, at least 110 days, at least 115 days, at least 120 days, or at least 1 year.

### Murine Models of Myotonic Dystrophy

To examine the therapeutic effects of an interfering RNA molecule described herein, or those of a vector encoding the same, an appropriate mouse model may be utilized. For example, the HSA (human skeletal actin) ^{LR} (long repeat) mouse model is an established model for myotonic dystrophy type 1 (see, e.g., Mankodi et al., Science 289:1769 (2000), the disclosure of which is incorporated herein by reference as it pertains to the HSA^{LR} mouse. These mice carry a human skeletal actin (hACTA1) transgene containing an expanded CTG region. Particularly, the hACTA1 transgene in HSA^{LR} mice contains 220 CTG repeats inserted in the 3′ UTR of the gene. Upon transcription, the hACTA1-CUGexp RNA transcript accumulates in nuclear foci in skeletal muscles and results in myotonia similar to that observed in human myotonic dystrophy type 1, for example, due to the binding of CUG repeat expansions to splicing factors and the sequestration of these splicing factors from pre-mRNA transcripts encoding genes that play an important role in regulating muscle function (see, e.g., Mankodi et al., Mol. Cell 10:35 (2002), and Lin et al., Hum. Mol. Genet. 15:2087 (2006), the disclosures of each of which are incorporated herein by reference as they pertain to the HSA^{LR} mouse). Thus, amelioration of myotonic dystrophy type I symptoms in the HSA^{LR} mouse by suppression of the expression of hACTA1 RNA transcripts harboring CUG expanded repeat regions may be a predictor of amelioration of similar symptoms in human patients by suppression of the expression of pathologic DMPK RNA transcripts. HSA^{LR} myotonic dystrophy type I mice can be generated using methods known in the art, for example, by insertion into the genome of FVB/N mice of a hACTA1 transgene with 250 CUG repeats in the 3′ UTR of human skeletal actin. The transgene is subsequently expressed in the mice as a CUG repeat expansion in hACTA1 RNA. This repeat-expanded RNA is retained in the nucleus, forming nuclear inclusions similar to those observed in human tissue samples of patients with myotonic dystrophy.

As described above, in the HSA^{LR} mouse model, the accumulation of expanded CUG RNA in the nucleus leads to the sequestration of poly(CUG)-binding splicing factor proteins, such as muscleblind-like protein (Miller et al., EMBO J. 19:4439 (2000)). This splicing factor, which controls alternative splicing of the SERCA1 gene, is thus sequestered in expanded CUG foci in HSA^{LR} mice. This sequestration triggers dysregulation of the alternative splicing of the SERCA1 gene. To evaluate the therapeutic effect of the interfering RNA molecules described herein, and/or of vectors encoding the same, these compositions may be designed so as to anneal to a region of the hACTA1 RNA transcript, for example, at a site distal from the CUG repeat expansion. This may be accomplished, for example, by designing an interfering RNA molecule that is at least 85% complementary (e.g., at least 85%, 90%, 95%, 96%, 97%, 98%, 99%, 99.9%, or 100% complementary) to a segment of hACTA1 RNA that does not overlap with the CUG repeat expansion region. The suppression of repeat-expanded hACTA1 RNA, and the concomitant increase in correctly spliced SERCA1 mRNA (and, thus, functional SERCA1 protein), can then be assessed using RNA and protein detection methods known in the art and described herein. For example, to monitor an increase in the expression of correctly spliced SERCA1 mRNA transcripts (e.g., SERCA1 mRNA transcripts containing exon 22, following administration of an interfering RNA molecule designed to anneal to, and suppress the expression of, pathologic hACTA1 RNA, total RNA may be purified from the HSA^{LR} mouse at one or more, or all, of the tibialis anterior, gastrocnemius, and quadriceps muscle using the RNeasy Lipid Tissue Mini Kit (Qiagen®), according to the manufacturer's instructions. RT-PCR may be performed with, for example, the SuperScript III One-Step RT-PCR System and Platinum Taq Polymerase (Invitrogen®), using gene-specific primers for cDNA synthesis and PCR amplification. The forward and reverse primers for SERCA1 have been described, for example, in Bennett and Swayze, Annu Rev. Pharmacol. 50:259-293 (2010)). PCR products may be separated on agarose gels, stained with SybrGreen I Nucleic Acid Gel Stain (Invitrogen®), and imaged using a Fujifilm LAS-3000 Intelligent Dark Box. Restoration of correct splicing of the SERCA1 gene by an interfering RNA molecule, or vector encoding the same, for example, in the tibialis anterior, gastrocnemius, and/or quadriceps muscles of the HSA^{LR} mouse, may be a predictor of the therapeutic efficacy of an interfering RNA molecule, or vector encoding the same, that anneals to a similar site on human DMPK RNA.

Additional murine models of myotonic dystrophy include LC15 mice, Line A, which are transgenic mice containing the entire human DMPK 3′UTR (developed by Wheeler et al, University of Rochester). These mice are the second generation of mice backcrossed to an FVB background. The DMPK transgene is expressed in these mice as a CUG repeat in the DMPK RNA transcript, which is retained in the nucleus, thereby forming nuclear inclusions similar to those observed in human tissue samples of patients with myotonic dystrophy. LC15 mice may express DMPK RNA transcripts containing from about 350 to about 400 CUG repeats. These mice display early signs of myotonic dystrophy type I and do not display any myotonia in their muscle tissues.

Yet another murine model of myotonic dystrophy that may be used to assess the therapeutic efficacy of an interfering RNA molecule described herein or a vector encoding the same is the DMSXL model. DMSXL mice are generated by way of successive breeding of mice having a high level of CTG repeat instability, and, as a result, DMSXL mice express DMPK RNA transcripts containing >1,000 CUG trinucleotide repeats in the 3′ UTR. DMSXL mice and methods for producing the same are described in detail, for example, in Gomes-Pereira et al., PLoS Genet. 3:e52 (2007), and Huguet et al., A, PLoS Genet. 8:e1003043 (2012), the disclosures of each of which are incorporated herein by reference in their entirety.

### Additional Disorders Characterized by Nuclear Retention of Repeat-Expanded RNA

In addition to myotonic dystrophy type I, disorders characterized by the expression and nuclear retention of RNA transcripts harboring expanded repeat regions and that may be treated using the compositions and methods described herein include myotonic dystrophy type II and amyotrophic lateral sclerosis, among others. In the case of myotonic dystrophy type II, patients may express a mutant version of the cellular nucleic acid binding protein (CNBP) gene (also known as the zinc finger protein 9 (ZNF9) gene) harboring a CCUG (SEQ ID NO: 164) repeat expansion. In cases of patients having amyotrophic lateral sclerosis, patients may express mutant versions of C9ORF72 harboring expanded GGGGCC (SEQ ID NO: 162) repeats. The nucleic acid sequence of several isoforms of C9ORF72 mRNA are shown in Table 3, below. In all instances, patients having these disorders may be treated by administration of interfering RNA molecules (or vectors encoding the same) that anneal to and suppress the expression of the mutant RNA transcript, thereby releasing RNA-binding proteins that would ordinarily bind other substrates but are instead sequestered by virtue of high-avidity binding to the repeat expansion regions in the mutant transcripts expressed by such patients. Methods for monitoring the reduction in the expression of nuclear-retained RNA transcripts, such as pathologic CNBP and C9ORF72 transcripts harboring CCUG (SEQ ID NO: 164) and GGGGCC (SEQ ID NO: 162) repeats, respectively, include various molecular biology techniques known in the art and described herein.

TABLE 3
Nucleic acid sequences of exemplary isoforms of C9ORF72 mRNA

| SEQ ID | NCBI Reference | |
|---|---|---|
| NO. | Sequence No. | Nucleic Acid Sequence |
| 163 | NM_001256054.2 | ACGUAACCUACGGUGUCCCGCUAGGAAAGAGAGGUGCG |
| | | UCAAACAGCGACAAGUUCCGCCCACGUAAAAGAUGACGC |
| | | UUGGUGUGUCAGCCGUCCCUGCUGCCCGGUUGCUUCUC |
| | | UUUUGGGGGCGGGGUCUAGCAAGAGCAGGUGUGGGUU |
| | | UAGGAGAUAUCUCCGGAGCAUUUGGAUAAUGUGACAGU |
| | | UGGAAUGCAGUGAUGUCGACUCUUUGCCCACCGCCAUC |
| | | UCCAGCUGUUGCCAAGACAGAGAUUGCUUUAAGUGGCAA |
| | | AUCACCUUUAUUAGCAGCUACUUUUGCUUACUGGGACAA |
| | | UAUUCUUGGUCCUAGAGUAAGGCACAUUUGGGCUCCAAA |
| | | GACAGAACAGGUACUUCUCAGUGAUGGAGAAAUAACUUU |
| | | UCUUGCCAACCACACUCUAAAUGGAGAAAUCCUUCGAAA |
| | | UGCAGAGAGUGGUGCUAUAGAUGUAAAGUUUUUUGUCU |
| | | UGUCUGAAAAGGGAGUGAUUAUUGUUUCAUUAAUCUUUG |
| | | AUGGAAACUGGAAUGGGGAUCGCAGCACAUAUGGACUAU |
| | | CAAUUAUACUUCCACAGACAGAACUUAGUUUCUACCUCC |
| | | CACUUCAUAGAGUGUGUGUUGAUAGAUUAACACAUAUAA |
| | | UCCGGAAAGGAAGAAUAUGGAUGCAUAAGGAAAGACAAG |
| | | AAAAUGUCCAGAAGAUUAUCUUAGAAGGCACAGAGAGAA |
| | | UGGAAGAUCAGGGUCAGAGUAUUAUUCCAAUGCUUACUG |
| | | GAGAAGUGAUUCCUGUAAUGGAACUGCUUUCAUCUAUGA |
| | | AAUCACACAGUGUUCCUGAAGAAAUAGAUAUAGCUGAUA |
| | | CAGUACUCAAUGAUGAUGAUAUUGGUGACAGCUGUCAUG |
| | | AAGGCUUUCUUCUCAAUGCCAUCAGCUCACACUUGCAAA |
| | | CCUGUGGCUGUUCCGUUGUAGUAGGUAGCAGUGCAGAG |
| | | AAAGUAAAUAAGAUAGUCAGAACAUUAUGCCUUUUUCUG |
| | | ACUCCAGCAGAGAGAAAAUGCUCCAGGUUAUGUGAAGCA |
| | | GAAUCAUCAUUUAAAUAUGAGUCAGGGCUCUUUGUACAA |
| | | GGCCUGCUAAAGGAUUCAACUGGAAGCUUUGUGCUGCC |
| | | UUUCCGGCAAGUCAUGUAUGCUCCAUAUCCCACCACACA |
| | | CAUAGAUGUGGAUGUCAAUACUGUGAAGCAGAUGCCACC |
| | | CUGUCAUGAACAUAUUUAUAAUCAGCGUAGAUACAUGAG |
| | | AUCCGAGCUGACAGCCUUCUGGAGAGCCACUUCAGAAGA |
| | | AGACAUGGCUCAGGAUACGAUCAUCUACACUGACGAAAG |
| | | CUUUACUCCUGAUUUGAAUAUUUUUCAAGAUGUCUUACA |
| | | CAGAGACACUCUAGUGAAAGCCUUCCUGGAUCAGGUCUU |
| | | UCAGCUGAAACCUGGCUUAUCUCUCAGAAGUACUUUCCU |
| | | UGCACAGUUUCUACUUGUCCUUCACAGAAAAGCCUUGAC |
| | | ACUAAUAAAAUAUAUAGAAGACGAUACGCAGAAGGGAAAA |
| | | AAGCCCUUUAAAUCUCUUCGGAACCUGAAGAUAGACCUU |
| | | GAUUUAACAGCAGAGGGCGAUCUUAACAUAAUAAUGGCU |
| | | CUGGCUGAGAAAAUUAAACCAGGCCUACACUCUUUUAUC |
| | | UUUGGAAGACCUUUCUACACUAGUGUGCAAGAACGAGAU |
| | | GUUCUAAUGACUUUUUAAAUGUGUAACUUAAUAAGCCUA |
| | | UUCCAUCACAAUCAUGAUCGCUGGUAAAGUAGCUCAGUG |
| | | GUGUGGGGAAACGUUCCCCUGGAUCAUACUCCAGAAUU |
| | | CUGCUCUCAGCAAUUGCAGUUAAGUAAGUUACACUACAG |
| | | UUCUCACAAGAGCCUGUGAGGGGAUGUCAGGUGCAUCA |
| | | UUACAUUGGGUGUCUCUUUUCCUAGAUUUAUGCUUUUG |
| | | GGAUACAGACCUAUGUUUACAAUAUAAUAAAUAUUAUUG |
| | | CUAUCUUUUAAAGAUAUAAUAAUAGGAUGUAAACUUGAC |
| | | CACAACUACUGUUUUUUUGAAAUACAUGAUUCAUGGUUU |
| | | ACAUGUGUCAAGGUGAAAUCUGAGUUGGCUUUUACAGAU |
| | | AGUUGACUUUCUAUCUUUUGGCAUUCUUUGGUGUGUAG |
| | | AAUUACUGUAAUACUUCUGCAAUCAACUGAAAACUAGAG |
| | | CCUUUAAAUGAUUUCAAUUCCACAGAAAGAAAGUGAGCU |
| | | UGAACAUAGGAUGAGCUUUAGAAAGAAAAUUGAUCAAGC |
| | | AGAUGUUUAAUUGGAAUUGAUUAUUAGAUCCUACUUUGU |
| | | GGAUUUAGUCCCUGGGAUUCAGUCUGUAGAAAUGUCUA |
| | | AUAGUUCUCUAUAGUCCUUGUUCCUGGUGAACCACAGUU |
| | | AGGGUGUUUUGUUUAUUUUAUUGUUCUUGCUAUUGUUG |
| | | AUAUUCUAUGUAGUUGAGCUCUGUAAAAGGAAAUUGUAU |
| | | UUUAUGUUUUAGUAAUUGUUGCCAACUUUUUAAAUUAAU |
| | | UUUCAUUAUUUUUGAGCCAAAUUGAAAUGUGCACCUCCU |
| | | GUGCCUUUUUUCUCCUUAGAAAAUCUAAUUACUUGGAAC |
| | | AAGUUCAGAUUUCACUGGUCAGUCAUUUUCAUCUUGUUU |
| | | UCUUCUUGCUAAGUCUUACCAUGUACCUGCUUUGGCAAU |
| | | CAUUGCAACUCUGAGAUUAUAAAAUGCCUUAGAGAAUAU |
| | | ACUAACUAAUAAGAUCUUUUUUUCAGAAACAGAAAAUAGU |
| | | UCCUUGAGUACUUCCUUCUUGCAUUUCUGCCUAUGUUU |
| | | UUGAAGUUGUUGCUGUUUGCCUGCAAUAGGCUAUAAGG |
| | | AAUAGCAGGAGAAAUUUUACUGAAGUGCUGUUUUCCUAG |
| | | GUGCUACUUUGGCAGAGCUAAGUUAUCUUUUGUUUUCU |
| | | UAAUGCGUUUGGACCAUUUUGCUGGCUAUAAAAUAACUG |
| | | AUUAAUAUAAUUCUAACACAAUGUUGACAUUGUAGUUAC |
| | | ACAAACACAAAUAAAUAUUUUAUUUAAAAUUCUGGAAGUA |
| | | AUAUAAAAGGGAAAAUAUAUUUAUAAGAAAGGGAUAAAG |
| | | GUAAUAGAGCCCUUCUGCCCCCCACCCACCAAAUUUACA |
| | | CAACAAAAUGACAUGUUCGAAUGUGAAAGGUCAUAAUAG |
| | | CUUUCCCAUCAUGAAUCAGAAAGAUGUGGACAGCUUGAU |
| | | GUUUUAGACAACCACUGAACUAGAUGACUGUUGUACUGU |
| | | AGCUCAGUCAUUUAAAAAAUAUAUAAAUACUACCUUGUA |
| | | GUGUCCCAUACUGUGUUUUUUACAUGGUAGAUUCUUAU |
| | | UUAAGUGCUAACUGGUUAUUUUCUUUGGCUGGUUUAUU |
| | | GUACUGUUAUACAGAAUGUAAGUUGUACAGUGAAAUAAG |
| | | UUAUUAAAGCAUGUGUAAACAUUGUUAUAUAUCUUUUCU |
| | | CCUAAAUGGAGAAUUUUGAAUAAAAUAUAUUUGAAAUUU |
| | | UAAAAAAAAAAAAAAAAAA |
| 165 | NM_018325.4 | GGGCGGGGCUGCGGUUGCGGUGCCUGCGCCCGCGGCG |
| | | GCGGAGGCGCAGGCGGUGGCGAGUGGAUAUCUCCGGA |
| | | GCAUUUGGAUAAUGUGACAGUUGGAAUGCAGUGAUGUC |
| | | GACUCUUUGCCCACCGCCAUCUCCAGCUGUUGCCAAGA |
| | | CAGAGAUUGCUUUAAGUGGCAAAUCACCUUUAUUAGCAG |
| | | CUACUUUUGCUUACUGGGACAAUAUUCUUGGUCCUAGA |
| | | GUAAGGCACAUUUGGGCUCCAAAGACAGAACAGGUACUU |
| | | CUCAGUGAUGGAGAAAUAACUUUUCUUGCCAACCACACU |
| | | CUAAAUGGAGAAAUCCUUCGAAAUGCAGAGAGUGGUGCU |
| | | AUAGAUGUAAAGUUUUUUGUCUUGUCUGAAAAGGGAGU |
| | | GAUUAUUGUUUCAUUAAUCUUUGAUGGAAACUGGAAUGG |
| | | GGAUCGCAGCACAUAUGGACUAUCAAUUAUACUUCCACA |
| | | GACAGAACUUAGUUUCUACCUCCCACUUCAUAGAGUGUG |
| | | UGUUGAUAGAUUAACACAUAUAAUCCGGAAAGGAAGAAU |
| | | AUGGAUGCAUAAGGAAAGACAAGAAAAUGUCCAGAAGAU |
| | | UAUCUUAGAAGGCACAGAGAGAAUGGAAGAUCAGGGUCA |
| | | GAGUAUUAUUCCAAUGCUUACUGGAGAAGUGAUUCCUGU |
| | | AAUGGAACUGCUUUCAUCUAUGAAAUCACACAGUGUUCC |
| | | UGAAGAAAUAGAUAUAGCUGAUACAGUACUCAAUGAUGA |
| | | UGAUAUUGGUGACAGCUGUCAUGAAGGCUUUCUUCUCA |
| | | AUGCCAUCAGCUCACACUUGCAAACCUGUGGCUGUUCC |
| | | GUUGUAGUAGGUAGCAGUGCAGAGAAAGUAAAUAAGAUA |
| | | GUCAGAACAUUAUGCCUUUUUCUGACUCCAGCAGAGAGA |
| | | AAAUGCUCCAGGUUAUGUGAAGCAGAAUCAUCAUUUAAA |
| | | UAUGAGUCAGGGCUCUUUGUACAAGGCCUGCUAAAGGA |
| | | UUCAACUGGAAGCUUUGUGCUGCCUUUCCGGCAAGUCA |
| | | UGUAUGCUCCAUAUCCCACCACACACAUAGAUGUGGAUG |
| | | UCAAUACUGUGAAGCAGAUGCCACCCUGUCAUGAACAUA |
| | | UUUAUAAUCAGCGUAGAUACAUGAGAUCCGAGCUGACAG |
| | | CCUUCUGGAGAGCCACUUCAGAAGAAGACAUGGCUCAG |
| | | GAUACGAUCAUCUACACUGACGAAAGCUUUACUCCUGAU |
| | | UUGAAUAUUUUUCAAGAUGUCUUACACAGAGACACUCUA |
| | | GUGAAAGCCUUCCUGGAUCAGGUCUUUCAGCUGAAACC |
| | | UGGCUUAUCUCUCAGAAGUACUUUCCUUGCACAGUUUCU |
| | | ACUUGUCCUUCACAGAAAAGCCUUGACACUAAUAAAAUA |
| | | UAUAGAAGACGAUACGCAGAAGGGAAAAAAGCCCUUUAA |
| | | AUCUCUUCGGAACCUGAAGAUAGACCUUGAUUUAACAGC |
| | | AGAGGGCGAUCUUAACAUAAUAAUGGCUCUGGCUGAGAA |
| | | AAUUAAACCAGGCCUACACUCUUUUAUCUUUGGAAGACC |
| | | UUUCUACACUAGUGUGCAAGAACGAGAUGUUCUAAUGAC |
| | | UUUUUAAAUGUGUAACUUAAUAAGCCUAUUCCAUCACAA |
| | | UCAUGAUCGCUGGUAAAGUAGCUCAGUGGUGUGGGGAA |
| | | ACGUUCCCCUGGAUCAUACUCCAGAAUUCUGCUCUCAGC |
| | | AAUUGCAGUUAAGUAAGUUACACUACAGUUCUCACAAGA |
| | | GCCUGUGAGGGGAUGUCAGGUGCAUCAUUACAUUGGGU |
| | | GUCUCUUUUCCUAGAUUUAUGCUUUUGGGAUACAGACC |
| | | UAUGUUUACAAUAUAAUAAAUAUUAUUGCUAUCUUUUAAA |
| | | GAUAUAAUAAUAGGAUGUAAACUUGACCACAACUACUGU |
| | | UUUUUUGAAAUACAUGAUUCAUGGUUUACAUGUGUCAAG |
| | | GUGAAAUCUGAGUUGGCUUUUACAGAUAGUUGACUUUC |
| | | UAUCUUUUGGCAUUCUUUGGUGUGUAGAAUUACUGUAA |
| | | UACUUCUGCAAUCAACUGAAAACUAGAGCCUUUAAAUGA |
| | | UUUCAAUUCCACAGAAAGAAAGUGAGCUUGAACAUAGGA |
| | | UGAGCUUUAGAAAGAAAAUUGAUCAAGCAGAUGUUUAAU |
| | | UGGAAUUGAUUAUUAGAUCCUACUUUGUGGAUUUAGUCC |
| | | CUGGGAUUCAGUCUGUAGAAAUGUCUAAUAGUUCUCUAU |
| | | AGUCCUUGUUCCUGGUGAACCACAGUUAGGGUGUUUUG |
| | | UUUAUUUUAUUGUUCUUGCUAUUGUUGAUAUUCUAUGUA |
| | | GUUGAGCUCUGUAAAAGGAAAUUGUAUUUUAUGUUUUAG |
| | | UAAUUGUUGCCAACUUUUUAAAUUAAUUUUCAUUAUUUU |
| | | UGAGCCAAAUUGAAAUGUGCACCUCCUGUGCCUUUUUUC |
| | | UCCUUAGAAAAUCUAAUUACUUGGAACAAGUUCAGAUUU |
| | | CACUGGUCAGUCAUUUUCAUCUUGUUUUCUUCUUGCUAA |
| | | GUCUUACCAUGUACCUGCUUUGGCAAUCAUUGCAACUCU |
| | | GAGAUUAUAAAAUGCCUUAGAGAAUAUACUAACUAAUAA |
| | | GAUCUUUUUUUCAGAAACAGAAAAUAGUUCCUUGAGUAC |
| | | UUCCUUCUUGCAUUUCUGCCUAUGUUUUUGAAGUUGUU |
| | | GCUGUUUGCCUGCAAUAGGCUAUAAGGAAUAGCAGGAG |
| | | AAAUUUUACUGAAGUGCUGUUUUCCUAGGUGCUACUUU |
| | | GGCAGAGCUAAGUUAUCUUUUGUUUUCUUAAUGCGUUU |
| | | GGACCAUUUUGCUGGCUAUAAAAUAACUGAUUAAUAUAA |
| | | UUCUAACACAAUGUUGACAUUGUAGUUACACAAACACAA |
| | | AUAAAUAUUUUAUUUAAAAUUCUGGAAGUAAUAUAAAAGG |
| | | GAAAAUAUAUUUAUAAGAAAGGGAUAAAGGUAAUAGAGC |
| | | CCUUCUGCCCCCCACCCACCAAAUUUACACAACAAAAUG |
| | | ACAUGUUCGAAUGUGAAAGGUCAUAAUAGCUUUCCCAUC |
| | | AUGAAUCAGAAAGAUGUGGACAGCUUGAUGUUUUAGACA |
| | | ACCACUGAACUAGAUGACUGUUGUACUGUAGCUCAGUCA |
| | | UUUAAAAAAUAUAUAAAUACUACCUUGUAGUGUCCCAUA |
| | | CUGUGUUUUUUACAUGGUAGAUUCUUAUUUAAGUGCUAA |
| | | CUGGUUAUUUUCUUUGGCUGGUUUAUUGUACUGUUAUA |
| | | CAGAAUGUAAGUUGUACAGUGAAAUAAGUUAUUAAAGCA |
| | | UGUGUAAACAUUGUUAUAUAUCUUUUCUCCUAAAUGGAG |
| | | AAUUUUGAAUAAAAUAUAUUUGAAAUUUUAAAAAAAAAAA |
| | | AAAAAAA |
| 166 | NM_145005.6 | ACGUAACCUACGGUGUCCCGCUAGGAAAGAGAGGUGCG |
| | | UCAAACAGCGACAAGUUCCGCCCACGUAAAAGAUGACGC |
| | | UUGAUAUCUCCGGAGCAUUUGGAUAAUGUGACAGUUGG |
| | | AAUGCAGUGAUGUCGACUCUUUGCCCACCGCCAUCUCCA |
| | | GCUGUUGCCAAGACAGAGAUUGCUUUAAGUGGCAAAUCA |
| | | CCUUUAUUAGCAGCUACUUUUGCUUACUGGGACAAUAUU |
| | | CUUGGUCCUAGAGUAAGGCACAUUUGGGCUCCAAAGACA |
| | | GAACAGGUACUUCUCAGUGAUGGAGAAAUAACUUUUCUU |
| | | GCCAACCACACUCUAAAUGGAGAAAUCCUUCGAAAUGCA |
| | | GAGAGUGGUGCUAUAGAUGUAAAGUUUUUUGUCUUGUC |
| | | UGAAAAGGGAGUGAUUAUUGUUUCAUUAAUCUUUGAUGG |
| | | AAACUGGAAUGGGGAUCGCAGCACAUAUGGACUAUCAAU |
| | | UAUACUUCCACAGACAGAACUUAGUUUCUACCUCCCACU |
| | | UCAUAGAGUGUGUGUUGAUAGAUUAACACAUAUAAUCCG |
| | | GAAAGGAAGAAUAUGGAUGCAUAAGGAAAGACAAGAAAA |
| | | UGUCCAGAAGAUUAUCUUAGAAGGCACAGAGAGAAUGGA |
| | | AGAUCAGGGUCAGAGUAUUAUUCCAAUGCUUACUGGAGA |
| | | AGUGAUUCCUGUAAUGGAACUGCUUUCAUCUAUGAAAUC |
| | | ACACAGUGUUCCUGAAGAAAUAGAUAUAGCUGAUACAGU |
| | | ACUCAAUGAUGAUGAUAUUGGUGACAGCUGUCAUGAAGG |
| | | CUUUCUUCUCAAGUAAGAAUUUUUCUUUUCAUAAAAGCU |
| | | GGAUGAAGCAGAUACCAUCUUAUGCUCACCUAUGACAAG |
| | | AUUUGGAAGAAAGAAAAUAACAGACUGUCUACUUAGAUU |
| | | GUUCUAGGGACAUUACGUAUUUGAACUGUUGCUUAAAUU |
| | | UGUGUUAUUUUUCACUCAUUAUAUUUCUAUAUAUAUUUG |
| | | GUGUUAUUCCAUUUGCUAUUUAAAGAAACCGAGUUUCCA |
| | | UCCCAGACAAGAAAUCAUGGCCCCUUGCUUGAUUCUGGU |
| | | UUCUUGUUUUACUUCUCAUUAAAGCUAACAGAAUCCUUU |
| | | CAUAUUAAGUUGUACUGUAGAUGAACUUAAGUUAUUUAG |
| | | GCGUAGAACAAAAUUAUUCAUAUUUAUACUGAUCUUUUU |
| | | CCAUCCAGCAGUGGAGUUUAGUACUUAAGAGUUUGUGC |
| | | CCUUAAACCAGACUCCCUGGAUUAAUGCUGUGUACCCGU |
| | | GGGCAAGGUGCCUGAAUUCUCUAUACACCUAUUUCCUCA |
| | | UCUGUAAAAUGGCAAUAAUAGUAAUAGUACCUAAUGUGU |
| | | AGGGUUGUUAUAAGCAUUGAGUAAGAUAAAUAAUAUAAA |
| | | GCACUUAGAACAGUGCCUGGAACAUAAAAACACUUAAUA |
| | | AUAGCUCAUAGCUAACAUUUCCUAUUUACAUUUCUUCUA |
| | | GAAAUAGCCAGUAUUUGUUGAGUGCCUACAUGUUAGUUC |
| | | CUUUACUAGUUGCUUUACAUGUAUUAUCUUAUAUUCUGU |
| | | UUUAAAGUUUCUUCACAGUUACAGAUUUUCAUGAAAUUU |
| | | UACUUUUAAUAAAAGAGAAGUAAAAGUAUAAAGUAUUCAC |
| | | UUUUAUGUUCACAGUCUUUUCCUUUAGGCUCAUGAUGG |
| | | AGUAUCAGAGGCAUGAGUGUGUUUAACCUAAGAGCCUUA |
| | | AUGGCUUGAAUCAGAAGCACUUUAGUCCUGUAUCUGUUC |
| | | AGUGUCAGCCUUUCAUACAUCAUUUUAAAUCCCAUUUGA |
| | | CUUUAAGUAAGUCACUUAAUCUCUCUACAUGUCAAUUUC |
| | | UUCAGCUAUAAAAUGAUGGUAUUUCAAUAAAUAAAUACAU |
| | | UAAUUAAAUGAUAUUAUACUGACUAAUUGGGCUGUUUUA |
| | | AGGCUCAAUAAGAAAAUUUCUGUGAAAGGUCUCUAGAAA |
| | | AUGUAGGUUCCUAUACAAAUAAAAGAUAACAUUGUGCUU |
| | | AUAAAAAAAA |

### Interfering RNA

Using the compositions and methods described herein, a patient having a spliceopathy and/or a disorder characterized by RNA dominance may be administered an interfering RNA molecule, a composition containing the same, or a vector encoding the same, so as to suppress the expression of a mutant RNA transcript containing an expanded repeat region. For example, in the case of myotonic dystrophy type I, the target RNA transcript to be suppressed is human DMPK RNA containing expanded CUG repeat regions in the 3′ UTR of the transcript. In the case of myotonic dystrophy type II, the target RNA transcript to be suppressed is human ZNF9 RNA containing expanded CCUG (SEQ ID NO: 164) repeat regions. In the case of amyotrophic lateral sclerosis, the target RNA transcript to be suppressed is human C9ORF72 RNA containing expanded GGGGCC (SEQ ID NO: 162) repeat regions.

Exemplary interfering RNA molecules that may be used in conjunction with the compositions and methods described herein for the treatment of RNA dominance disorders, such as myotonic dystrophy type I and others, are siRNA molecules, miRNA molecules, and shRNA molecules, among others. In the case of siRNA molecules, the siRNA may be single stranded or double stranded. miRNA molecules, in contrast, are single-stranded molecules that form a hairpin, thereby adopting a hydrogen-bonded structure reminiscent of a nucleic acid duplex. In either case, the interfering RNA may contain an antisense or “guide” strand that anneals (e.g., by way of complementarity) to the repeat-expanded mutant RNA target. The interfering RNA may also contain a “passenger” strand that is complementary to the guide strand and, thus, may have the same nucleic acid sequence as the RNA target.

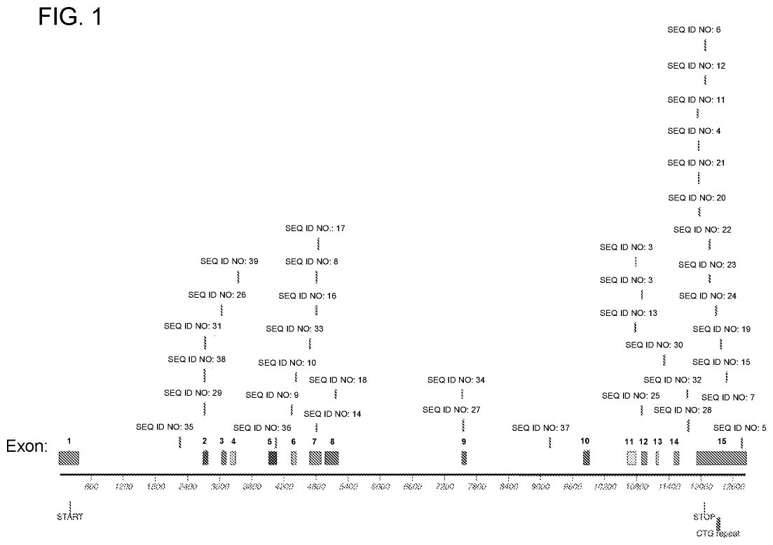

Exemplary interfering RNA molecules that anneal to mutant DMPK containing expanded CUG repeat motifs and that may be used in conjunction with the compositions and methods described herein for the treatment of myotonic dystrophy type I are shown in Table 4, below. A graphical representation of the sites on a target DMPK RNA transcript to which the following interfering RNA molecules anneal by way of sequence complementarity is shown in FIG. 1.

TABLE 4
Exemplary RNAi molecules useful for suppressing mutant DMPK
RNA expression

| SEQ ID | Type of Interfering | |
|---|---|---|
| NO. | RNA | Nucleic Acid Sequence |
| 3 | siRNA (Identical to | CGCCAGUCAACUACGCGA |
| | target sequence) | |
| 4 | siRNA (Identical to | CCUGCUCCUGUUCGCCGUU |
| | target sequence) | |
| 5 | siRNA (Identical to | CCCGACAUUCCUCGGUAUU |
| | target sequence) | |
| 6 | siRNA (Identical to | CCUAGAACUGUCUUCGACU |
| | target sequence) | |
| 7 | siRNA (Identical to | CCUAUCGUUGGUUCGCAAA |
| | target sequence) | |
| 8 | siRNA (Identical to | GCUGGGUGUAUUCGCCUAU |
| | target sequence) | |
| 9 | siRNA (Identical to | GGACAUCAAACCCGACAAC |
| | target sequence) | |
| 10 | siRNA (Identical to | GCGGGCAGAUGGAACGGUG |
| | target sequence) | |
| 11 | siRNA (Identical to | GGCCUAUCGGAGGCGCUUU |
| | target sequence) | |
| 12 | siRNA (Identical to | CCCUAGAACUGUCUUCGAC |
| | target sequence) | |
| 13 | siRNA (Identical to | GGACAACCAGAACUUCGCC |
| | target sequence) | |
| 14 | siRNA (Identical to | CGCUGGGUGUAUUCGCCUA |
| | target sequence) | |
| 15 | siRNA (Identical to | CACCUAUCGUUGGUUCGCA |
| | target sequence) | |
| 16 | siRNA (Identical to | GGCGCUGGGUGUAUUCGCC |
| | target sequence) | |
| 17 | siRNA (Identical to | CGCCCUUCUACGCGGAUUC |
| | target sequence) | |
| 18 | siRNA (Identical to | CGAAGGUGCCACCGACACA |
| | target sequence) | |
| 19 | siRNA (Identical to | CCAUUUCUUUCUUUCGGCC |
| | target sequence) | |
| 20 | siRNA (Identical to | GCCGUUGUUCUGUCUCGUG |
| | target sequence) | |
| 21 | siRNA (Identical to | GCUCCUGUUCGCCGUUGUU |
| | target sequence) | |
| 22 | siRNA (Identical to | GCCAGUUCACAACCGCUCC |
| | target sequence) | |
| 23 | siRNA (Identical to | CCAGUUCACAACCGCUCCG |
| | target sequence) | |
| 24 | siRNA (Identical to | GGUCCUUGUAGCCGGGAAU |
| | target sequence) | |
| 25 | siRNA (Identical to | CCACAGUCAACUACGCGAG |
| | target sequence) | |
| 26 | siRNA (Identical to | CGACCUCAGGUAGCGGUAG |
| | target sequence) | |
| 27 | siRNA (Identical to | CGGGAAGGUGCGCCGCUAG |
| | target sequence) | |
| 28 | siRNA (Identical to | GGGAUUCGAGGCGUGCGAG |
| | target sequence) | |
| 29 | siRNA (Identical to | GGCUUAAGGAGGUCCGACU |
| | target sequence) | |
| 30 | siRNA (Identical to | CCCUACGUCUUUGCGACUU |
| | target sequence) | |
| 31 | siRNA (Identical to | CGACUGCAGAGGGACGACU |
| | target sequence) | |
| 32 | siRNA (Identical to | CCAAUGACGAGUUCGGACG |
| | target sequence) | |
| 33 | siRNA (Identical to | GGUCCAGGUGCGGUCGCUG |
| | target sequence) | |
| 34 | siRNA (Identical to | GCAGGAGACACUGUCGGAC |
| | target sequence) | |
| 35 | siRNA (Identical to | CCCACGUCUGGGCCGUUAC |
| | target sequence) | |
| 36 | siRNA (Identical to | GCACCGGCUUGGCUACGUG |
| | target sequence) | |
| 37 | siRNA (Identical to | GCUUGAUUCUGAACCGCUG |
| | target sequence) | |
| 38 | siRNA (Identical to | GCUUAAGGAGGUCCGACUG |
| | target sequence) | |
| 39 | siRNA (Identical to | CGUGCUGUCACUGGACGAG |
| | target sequence) | |
| 40 | miRNA (passenger | AGCGCCAGUCAACUACGCGAG |
| | strand) | |
| 41 | miRNA (passenger | CCCCUGCUCCUGUUCGCCGUU |
| | strand) | |
| 42 | miRNA (passenger | AGCCCGACAUUCCUCGGUAUU |
| | strand) | |
| 43 | miRNA (passenger | ACCCUAGAACUGUCUUCGAUU |
| | strand) | |
| 44 | miRNA (passenger | ACCCUAUCGUUGGUUCGCAAA |
| | strand) | |
| 45 | miRNA (passenger | ACGCUGGGUGUAUUCGCCUAU |
| | strand) | |
| 46 | miRNA (passenger | CGGGACAUCAAACCCGACAAU |
| | strand) | |
| 47 | miRNA (passenger | ACGCGGGCAGAUGGAACGGUG |
| | strand) | |
| 48 | miRNA (passenger | ACGGCCUAUCGGAGGCGCUUU |
| | strand) | |
| 49 | miRNA (passenger | AGCCCUAGAACUGUCUUCGAU |
| | strand) | |
| 50 | miRNA (passenger | ACGGACAACCAGAACUUCGUU |
| | strand) | |
| 51 | miRNA (passenger | AGCGCUGGGUGUAUUCGCUUA |
| | strand) | |
| 52 | miRNA (passenger | ACCACCUAUCGUUGGUUCGUA |
| | strand) | |
| 53 | miRNA (passenger | CGGGCGCUGGGUGUAUUCGUU |
| | strand) | |
| 54 | miRNA (passenger | ACCGCCCUUCUACGCGGAUUU |
| | strand) | |
| 55 | miRNA (passenger | CGCGAAGGUGCCACCGACAUA |
| | strand) | |
| 56 | miRNA (passenger | ACCCAUUUCUUUCUUUCGGUU |
| | strand) | |
| 57 | miRNA (passenger | ACGCCGUUGUUCUGUCUCGUG |
| | strand) | |
| 58 | miRNA (passenger | ACGCUCCUGUUCGCCGUUGUU |
| | strand) | |
| 59 | miRNA (passenger | ACGCCAGUUCACAACCGCUUU |
| | strand) | |
| 60 | miRNA (passenger | CGCCAGUUCACAACCGCUUUG |
| | strand) | |
| 61 | miRNA (passenger | CGGGUCCUUGUAGCCGGGAAU |
| | strand) | |
| 62 | miRNA (passenger | ACCCACAGUCAACUACGCGAG |
| | strand) | |
| 63 | miRNA (passenger | ACCGACCUCAGGUAGCGGUAG |
| | strand) | |
| 64 | miRNA (passenger | CGCGGGAAGGUGCGCCGCUAG |
| | strand) | |
| 65 | miRNA (passenger | ACGGGAUUCGAGGCGUGCGAG |
| | strand) | |
| 66 | miRNA (passenger | ACGGCUUAAGGAGGUCCGAUU |
| | strand) | |
| 67 | miRNA (passenger | AGCCCUACGUCUUUGCGAUUU |
| | strand) | |
| 68 | miRNA (passenger | CCCGACUGCAGAGGGACGAUU |
| | strand) | |
| 69 | miRNA (passenger | CGCCAAUGACGAGUUCGGAUG |
| | strand) | |
| 70 | miRNA (passenger | CGGGUCCAGGUGCGGUCGUUG |
| | strand) | |
| 71 | miRNA (passenger | ACGCAGGAGACACUGUCGGAU |
| | strand) | |
| 72 | miRNA (passenger | CGCCCACGUCUGGGCCGUUAU |
| | strand) | |
| 73 | miRNA (passenger | ACGCACCGGCUUGGCUACGUG |
| | strand) | |
| 74 | miRNA (passenger | ACGCUUGAUUCUGAACCGUUG |
| | strand) | |
| 75 | miRNA (passenger | CGGCUUAAGGAGGUCCGAUUG |
| | strand) | |
| 76 | miRNA (passenger | ACCGUGCUGUCACUGGACGAG |
| | strand) | |
| 77 | miRNA (guide strand) | UUCGCGUAGUUGACUGGCGCC |
| 78 | miRNA (guide strand) | AACGGCGAACAGGAGCAGGGC |
| 79 | miRNA (guide strand) | AAUACCGAGGAAUGUCGGGCC |
| 80 | miRNA (guide strand) | AGUCGAAGACAGUUCUAGGGC |
| 81 | miRNA (guide strand) | UUUGCGAACCAACGAUAGGGC |
| 82 | miRNA (guide strand) | AUAGGCGAAUACACCCAGCGC |
| 83 | miRNA (guide strand) | GUUGUCGGGUUUGAUGUCCCA |
| 84 | miRNA (guide strand) | UACCGUUCCAUCUGCCCGCGC |
| 85 | miRNA (guide strand) | AAAGCGCCUCCGAUAGGCCGC |
| 86 | miRNA (guide strand) | GUCGAAGACAGUUCUAGGGCC |
| 87 | miRNA (guide strand) | GGCGAAGUUCUGGUUGUCCGC |
| 88 | miRNA (guide strand) | UAGGCGAAUACACCCAGCGCC |
| 89 | miRNA (guide strand) | UGCGAACCAACGAUAGGUGGC |
| 90 | miRNA (guide strand) | GGCGAAUACACCCAGCGCCCA |
| 91 | miRNA (guide strand) | GAAUCCGCGUAGAAGGGCGGC |
| 92 | miRNA (guide strand) | UGUGUCGGUGGCACCUUCGCA |
| 93 | miRNA (guide strand) | GGCCGAAAGAAAGAAAUGGGC |
| 94 | miRNA (guide strand) | UACGAGACAGAACAACGGCGC |
| 95 | miRNA (guide strand) | AACAACGGCGAACAGGAGCGC |
| 96 | miRNA (guide strand) | GGAGCGGUUGUGAACUGGCGC |
| 97 | miRNA (guide strand) | UGGAGCGGUUGUGAACUGGCA |
| 98 | miRNA (guide strand) | AUUCCCGGCUACAAGGACCCU |
| 99 | miRNA (guide strand) | UUCGCGUAGUUGACUGUGG |
| 100 | miRNA (guide strand) | UUACCGCUACCUGAGGUCGGC |
| 101 | miRNA (guide strand) | UUAGCGGCGCACCUUCCCGCA |
| 102 | miRNA (guide strand) | UUCGCACGCCUCGAAUCCCGC |
| 103 | miRNA (guide strand) | AGUCGGACCUCCUUAAGCCGC |
| 104 | miRNA (guide strand) | AAGUCGCAAAGACGUAGGGCC |
| 105 | miRNA (guide strand) | AGUCGUCCCUCUGCAGUCGGA |
| 106 | miRNA (guide strand) | UGUCCGAACUCGUCAUUGGCA |
| 107 | miRNA (guide strand) | UAGCGACCGCACCUGGACCCA |
| 108 | miRNA (guide strand) | GUCCGACAGUGUCUCCUGCGC |
| 109 | miRNA (guide strand) | GUAACGGCCCAGACGUGGGCA |
| 110 | miRNA (guide strand) | UACGUAGCCAAGCCGGUGCGC |
| 111 | miRNA (guide strand) | UAGCGGUUCAGAAUCAAGCGC |
| 112 | miRNA (guide strand) | UAGUCGGACCUCCUUAAGCCA |
| 113 | miRNA (guide strand) | UUCGUCCAGUGACAGCACGGC |
| 114 | miRNA | AAAACUCGAGUGAGCGCGGGCGCUGGGUGUAUUCGU |
| | | UACUGUAAAGCCACAGAUG |
| 115 | miRNA | AAAACUCGAGUGAGCGACCGCCCUUCUACGCGGAUUU |
| | | ACUGUAAAGCCACAGAUG |
| 116 | miRNA | AAAACUCGAGUGAGCGCGCGAAGGUGCCACCGACAUA |
| | | ACUGUAAAGCCACAGAUG |
| 117 | miRNA | AAAACUCGAGUGAGCGACCCAUUUCUUUCUUUCGGUU |
| | | ACUGUAAAGCCACAGAUG |
| 118 | miRNA | AAAACUCGAGUGAGCGACGCCGUUGUUCUGUCUCGU |
| | | GACUGUAAAGCCACAGAUG |
| 119 | miRNA | AAAACUCGAGUGAGCGACGCUCCUGUUCGCCGUUGU |
| | | UACUGUAAAGCCACAGAUG |
| 120 | miRNA | AAAACUCGAGUGAGCGACGCCAGUUCACAACCGCUUU |
| | | ACUGUAAAGCCACAGAUG |
| 121 | miRNA | AAAACUCGAGUGAGCGCGCCAGUUCACAACCGCUUUG |
| | | ACUGUAAAGCCACAGAUG |
| 122 | miRNA | AAAACUCGAGUGAGCGCGGGUCCUUGUAGCCGGGAA |
| | | UACUGUAAAGCCACAGAUG |
| 123 | miRNA | UUUUACUAGUAGGCGUGGGCGCUGGGUGUAUUCGCC |
| | | AGCAUCUGUGGCUUUACAG |
| 124 | miRNA | UUUUACUAGUAGGCGGCCGCCCUUCUACGCGGAUUC |
| | | AGCAUCUGUGGCUUUACAG |
| 125 | miRNA | UUUUACUAGUAGGCGUGCGAAGGUGCCACCGACACAA |
| | | GCAUCUGUGGCUUUACAG |
| 126 | miRNA | UUUUACUAGUAGGCGGCCCAUUUCUUUCUUUCGGCC |
| | | AGCAUCUGUGGCUUUACAG |
| 127 | miRNA | UUUUACUAGUAGGCGGCGCCGUUGUUCUGUCUCGUA |
| | | AGCAUCUGUGGCUUUACAG |
| 128 | miRNA | UUUUACUAGUAGGCGGCGCUCCUGUUCGCCGUUGUU |
| | | AGCAUCUGUGGCUUUACAG |
| 129 | miRNA | UUUUACUAGUAGGCGGCGCCAGUUCACAACCGCUCCA |
| | | GCAUCUGUGGCUUUACAG |
| 130 | miRNA | UUUUACUAGUAGGCGUGCCAGUUCACAACCGCUCCAA |
| | | GCAUCUGUGGCUUUACAG |
| 131 | miRNA | UUUUACUAGUAGGCGUGGGUCCUUGUAGCCGGGAAU |
| | | AGCAUCUGUGGCUUUACAG |
| 132 | miRNA | AAAACUCGAGUGAGCGACCCACAGUCAACUACGCGAG |
| | | ACUGUAAAGCCACAGAUG |
| 133 | miRNA | UUUUACUAGUAGGCGGCCCACAGUCAACUACGCGAAA |
| | | GCAUCUGUGGCUUUACAG |
| 134 | miRNA | AAAACUCGAGUGAGCGACCGACCUCAGGUAGCGGUAG |
| | | ACUGUAAAGCCACAGAUG |
| 135 | miRNA | UUUUACUAGUAGGCGGCCGACCUCAGGUAGCGGUAA |
| | | AGCAUCUGUGGCUUUACAG |
| 136 | miRNA | AAAACUCGAGUGAGCGCGCGGGAAGGUGCGCCGCUA |
| | | GACUGUAAAGCCACAGAUG |
| 137 | miRNA | UUUUACUAGUAGGCGUGCGGGAAGGUGCGCCGCUAA |
| | | AGCAUCUGUGGCUUUACAG |
| 138 | miRNA | AAAACUCGAGUGAGCGACGGGAUUCGAGGCGUGCGA |
| | | GACUGUAAAGCCACAGAUG |
| 139 | miRNA | UUUUACUAGUAGGCGGCGGGAUUCGAGGCGUGCGAA |
| | | AGCAUCUGUGGCUUUACAG |
| 140 | miRNA | AAAACUCGAGUGAGCGACGGCUUAAGGAGGUCCGAUU |
| | | ACUGUAAAGCCACAGAUG |
| 141 | miRNA | UUUUACUAGUAGGCGGCGGCUUAAGGAGGUCCGACU |
| | | AGCAUCUGUGGCUUUACAG |
| 142 | miRNA | AAAACUCGAGUGAGCGAGCCCUACGUCUUUGCGAUUU |
| | | ACUGUAAAGCCACAGAUG |
| 143 | miRNA | UUUUACUAGUAGGCGGGCCCUACGUCUUUGCGACUU |
| | | AGCAUCUGUGGCUUUACAG |
| 144 | miRNA | AAAACUCGAGUGAGCGCCCGACUGCAGAGGGACGAUU |
| | | ACUGUAAAGCCACAGAUG |
| 145 | miRNA | UUUUACUAGUAGGCGUCCGACUGCAGAGGGACGACU |
| | | AGCAUCUGUGGCUUUACAG |
| 146 | miRNA | AAAACUCGAGUGAGCGCGCCAAUGACGAGUUCGGAUG |
| | | ACUGUAAAGCCACAGAUG |
| 147 | miRNA | UUUUACUAGUAGGCGUGCCAAUGACGAGUUCGGACAA |
| | | GCAUCUGUGGCUUUACAG |
| 148 | miRNA | AAAACUCGAGUGAGCGCGGGUCCAGGUGCGGUCGUU |
| | | GACUGUAAAGCCACAGAUG |
| 149 | miRNA | UUUUACUAGUAGGCGUGGGUCCAGGUGCGGUCGCUA |
| | | AGCAUCUGUGGCUUUACAG |
| 150 | miRNA | AAAACUCGAGUGAGCGACGCAGGAGACACUGUCGGAU |
| | | ACUGUAAAGCCACAGAUG |
| 151 | miRNA | UUUUACUAGUAGGCGGCGCAGGAGACACUGUCGGAC |
| | | AGCAUCUGUGGCUUUACAG |
| 152 | miRNA | AAAACUCGAGUGAGCGCGCCCACGUCUGGGCCGUUA |
| | | UACUGUAAAGCCACAGAUG |
| 153 | miRNA | UUUUACUAGUAGGCGUGCCCACGUCUGGGCCGUUAC |
| | | AGCAUCUGUGGCUUUACAG |
| 154 | miRNA | AAAACUCGAGUGAGCGACGCACCGGCUUGGCUACGU |
| | | GACUGUAAAGCCACAGAUG |
| 155 | miRNA | UUUUACUAGUAGGCGGCGCACCGGCUUGGCUACGUA |
| | | AGCAUCUGUGGCUUUACAG |
| 156 | miRNA | AAAACUCGAGUGAGCGACGCUUGAUUCUGAACCGUUG |
| | | ACUGUAAAGCCACAGAUG |
| 157 | miRNA | UUUUACUAGUAGGCGGCGCUUGAUUCUGAACCGCUAA |
| | | GCAUCUGUGGCUUUACAG |
| 158 | miRNA | AAAACUCGAGUGAGCGCGGCUUAAGGAGGUCCGAUU |
| | | GACUGUAAAGCCACAGAUG |
| 159 | miRNA | UUUUACUAGUAGGCGUGGCUUAAGGAGGUCCGACUA |
| | | AGCAUCUGUGGCUUUACAG |
| 160 | miRNA | AAAACUCGAGUGAGCGACCGUGCUGUCACUGGACGA |
| | | GACUGUAAAGCCACAGAUG |
| 161 | miRNA | UUUUACUAGUAGGCGGCCGUGCUGUCACUGGACGAA |
| | | AGCAUCUGUGGCUUUACAG |

Exemplary miRNA constructs useful in conjunction with the compositions and methods described herein are those that have a combination of passenger and guide strands shown in Table 5, below.

TABLE 5
Exemplary anti-DMPK miRNA guide strand/passenger
strand combinations

| Entry | siRNA on which miRNA | miRNA | miRNA |
|---|---|---|---|
| No. | construct is based | Passenger Strand | Guide Strand |
| 1 | SEQ ID NO: 3 | SEQ ID NO: 40 | SEQ ID NO: 77 |
| 2 | SEQ ID NO: 4 | SEQ ID NO: 41 | SEQ ID NO: 78 |
| 3 | SEQ ID NO: 5 | SEQ ID NO: 42 | SEQ ID NO: 79 |
| 4 | SEQ ID NO: 6 | SEQ ID NO: 43 | SEQ ID NO: 80 |
| 5 | SEQ ID NO: 7 | SEQ ID NO: 44 | SEQ ID NO: 81 |
| 6 | SEQ ID NO: 8 | SEQ ID NO: 45 | SEQ ID NO: 82 |
| 7 | SEQ ID NO: 9 | SEQ ID NO: 46 | SEQ ID NO: 83 |
| 8 | SEQ ID NO: 10 | SEQ ID NO: 47 | SEQ ID NO: 84 |
| 9 | SEQ ID NO: 11 | SEQ ID NO: 48 | SEQ ID NO: 85 |
| 10 | SEQ ID NO: 12 | SEQ ID NO: 49 | SEQ ID NO: 86 |
| 11 | SEQ ID NO: 13 | SEQ ID NO: 50 | SEQ ID NO: 87 |
| 12 | SEQ ID NO: 14 | SEQ ID NO: 51 | SEQ ID NO: 88 |
| 13 | SEQ ID NO: 15 | SEQ ID NO: 52 | SEQ ID NO: 89 |
| 14 | SEQ ID NO: 16 | SEQ ID NO: 53 | SEQ ID NO: 90 |
| 15 | SEQ ID NO: 17 | SEQ ID NO: 54 | SEQ ID NO: 91 |
| 16 | SEQ ID NO: 18 | SEQ ID NO: 55 | SEQ ID NO: 92 |
| 17 | SEQ ID NO: 19 | SEQ ID NO: 56 | SEQ ID NO: 93 |
| 18 | SEQ ID NO: 20 | SEQ ID NO: 57 | SEQ ID NO: 94 |
| 19 | SEQ ID NO: 21 | SEQ ID NO: 58 | SEQ ID NO: 95 |
| 20 | SEQ ID NO: 22 | SEQ ID NO: 59 | SEQ ID NO: 96 |
| 21 | SEQ ID NO: 23 | SEQ ID NO: 60 | SEQ ID NO: 97 |
| 22 | SEQ ID NO: 24 | SEQ ID NO: 61 | SEQ ID NO: 98 |
| 23 | SEQ ID NO: 25 | SEQ ID NO: 62 | SEQ ID NO: 99 |
| 24 | SEQ ID NO: 26 | SEQ ID NO: 63 | SEQ ID NO: 100 |
| 25 | SEQ ID NO: 27 | SEQ ID NO: 64 | SEQ ID NO: 101 |
| 26 | SEQ ID NO: 28 | SEQ ID NO: 65 | SEQ ID NO: 102 |
| 27 | SEQ ID NO: 29 | SEQ ID NO: 66 | SEQ ID NO: 103 |
| 28 | SEQ ID NO: 30 | SEQ ID NO: 67 | SEQ ID NO: 104 |
| 29 | SEQ ID NO: 31 | SEQ ID NO: 68 | SEQ ID NO: 105 |
| 30 | SEQ ID NO: 32 | SEQ ID NO: 69 | SEQ ID NO: 106 |
| 31 | SEQ ID NO: 33 | SEQ ID NO: 70 | SEQ ID NO: 107 |
| 32 | SEQ ID NO: 34 | SEQ ID NO: 71 | SEQ ID NO: 108 |
| 33 | SEQ ID NO: 35 | SEQ ID NO: 72 | SEQ ID NO: 109 |
| 34 | SEQ ID NO: 36 | SEQ ID NO: 73 | SEQ ID NO: 110 |
| 35 | SEQ ID NO: 37 | SEQ ID NO: 74 | SEQ ID NO: 111 |
| 36 | SEQ ID NO: 38 | SEQ ID NO: 75 | SEQ ID NO: 112 |
| 37 | SEQ ID NO: 39 | SEQ ID NO: 76 | SEQ ID NO: 113 |

### Vectors for Delivery of Interfering RNA

### Viral Vectors for Interfering RNA Delivery

Viral genomes provide a rich source of vectors that can be used for the efficient delivery of a gene of interest into the genome of a target cell in a patient (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents in order to induce gene integration. Examples of viral vectors that may be used in conjunction with the compositions and methods described herein are AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses that may be used in conjunction with the compositions and methods described herein include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### AAV Vectors for Interfering RNA Delivery

In some embodiments, interfering RNA constructs described herein are incorporated into recombinant AAV (rAAV) vectors in order to facilitate their introduction into a cell, such as a target cardiac cell (e.g., a muscle cell) in a patient. rAAV vectors useful in the conjunction with the compositions and methods described herein include recombinant nucleic acid constructs that contain (1) a transgene encoding an interfering RNA construct described herein (such as an siRNA, shRNA, or miRNA described herein) and (2) nucleic acids that facilitate and expression of the heterologous genes. The viral nucleic acids may include those sequences of AAV that are required in cis for replication and packaging (e.g., functional ITRs) of the DNA into a virion. Such rAAV vectors may also contain marker or reporter genes. Useful rAAV vectors include those having one or more of the naturally-occurring AAV genes deleted in whole or in part, but retain functional flanking ITR sequences. The AAV ITRs may be of any serotype (e.g., derived from serotype 2) suitable for a particular application. Methods for using rAAV vectors are described, for example, in Tal et al., J. Biomed. Sci. 7:279-291 (2000), and Monahan and Samulski, Gene Delivery 7:24-30 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791-801 (2002) and Bowles et al., J. Virol. 77:423-432 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8 and 9. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J. Virol. 74:1524-1532 (2000); Halbert et al., J. Virol. 75:6615-6624 (2001); and Auricchio et al., Hum. Molec. Genet. 10:3075-3081 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV2) pseudotyped with a capsid gene derived from a serotype other than the given serotype (e.g., AAV1, AAV3, AAV4, AAV5, AAV6, AAV7, AAV8, or AAV9, among others). For example, a representative pseudotyped vector is an AAV2 vector encoding a therapeutic protein pseudotyped with a capsid gene derived from AAV serotype 8 or AAV serotype 9. Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45 (2000). Other rAAV virions that can be used in methods of the invention include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Additional Methods for the Delivery of Interfering RNA

### Transfection Techniques

Techniques that can be used to introduce a transgene, such as a transgene encoding an interfering RNA construct described herein, into a target cell (e.g., a target cell from or within a human patient suffering from RNA dominance) are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Topics in Current Chemistry 228:227 (2003), the disclosure of which is incorporated herein by reference) and diethylaminoethyl (DEAE)-dextran, the use of which as a transfection agent is described in detail, for example, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for example, in US 2010/0227406, the disclosure of which is incorporated herein by reference.

Another useful tool for inducing the uptake of exogenous nucleic acids by target cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Genes Encoding Interfering RNA by Gene Editing

In addition to the above, a variety of tools have been developed that can be used for the incorporation of a transgene, such as a transgene encoding an interfering RNA construct described herein, into a target cell, and particularly into a human cell. One such method that can be used for incorporating polynucleotides encoding interfering RNA constructs into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the transgene of interest can be integrated into the genome of a mammalian cell by transposase-catalyzed cleavage of similar excision sites that exist within the nuclear genome of the cell. This allows the transgene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target transgenes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a transgene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nature Reviews Genetics 11:636 (2010); and in Joung et al., Nature Reviews Molecular Cell Biology 14:49 (2013), the disclosure of each of which are incorporated herein by reference as they pertain to compositions and methods for genome editing.

Additional genome editing techniques that can be used to incorporate polynucleotides encoding target transgenes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target transgene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Methods of Detecting RNA Transcript Expression

The expression level of a pathological RNA transcript, such as a DMPK RNA transcript harboring an expanded CUG trinucleotide repeat or a C9ORF72 RNA transcript harboring an expanded GGGGCC (SEQ ID NO: 162) hexanucleotide can be ascertained, for example, by a variety of nucleic acid detection techniques. Additionally or alternatively, RNA transcript expression can be inferred by evaluating the concentration or relative abundance of an encoded protein produced by translation of the RNA transcript. Protein concentrations can also be assessed, for example, using functional assays. Using these techniques, a reduction in the concentration of pathological RNA transcripts in response to the compositions and methods described herein can be observed, while monitoring the expression of the encoded protein. The sections that follow describe exemplary techniques that can be used to measure the expression level of a pathological RNA transcript and its downstream protein product. RNA transcript expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of RNAs.

### Nucleic Acid Detection

Nucleic acid-based methods for detection of RNA transcript expression include imaging-based techniques (e.g., Northern blotting or Southern blotting), which may be used in conjunction with cells obtained from a patient following administration of, for example, a vector encoding an interfering RNA described herein or a composition containing such an interfering RNA construct. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, N.Y. 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

RNA detection techniques that may be used in conjunction with the compositions and methods described herein to evaluate the suppression of RNA transcripts harboring expanded nucleotide repeat regions, such as DMPK RNA transcripts harboring expanded CUG trinucleotide repeats and C9ORF72 RNA transcripts harboring expanded GGGGCC (SEQ ID NO: 162) hexanucleotide repeats, further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For example, transgene expression at the mRNA level may be determined using RNA-Seq (e.g., as described in Mortazavi et al., Nat. Methods 5:621-628 (2008), the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology®). Then, the cDNA is converted into a molecular library for sequencing by addition of sequence adapters for each library (e.g., from Illumina®/Solexa), and the resulting 50-100 nucleotide reads are mapped onto the genome.

RNA expression levels may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of a particular RNA transcript, such as a DMPK RNA transcript harboring an expanded CUG trinucleotide repeat or a C9ORF72 RNA transcript harboring an expanded GGGGCC (SEQ ID NO: 162) hexanucleotide repeat. In such assays, the nucleic acid sequence of the transcript acts as a template in an amplification reaction (for example, PCR, such as qPCR). In a quantitative amplification, the amount of amplification product is proportional to the amount of template in the original sample. Comparison to appropriate controls provides a measure of the expression level of the transcript of interest, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of RNA transcript expression as described herein can be determined, for example, by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of an RNA construct may also be inferred by analyzing expression of the protein encoded by the construct. Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize transgene expression in a cell from a patient (e.g., a human patient) following delivery of the transgene. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Pharmaceutical Compositions

The interfering RNA constructs, as well as the vectors and compositions encoding or containing these constructs, may be incorporated into a vehicle for administration into a patient, such as a human patient suffering from RNA dominance, as described herein. Pharmaceutical compositions containing vectors, such as viral vectors, that encode an interfering RNA construct described herein can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Mixtures of the nucleic acids and viral vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intrathecal, intracerebroventricular, intraparenchymal, intracisternal, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

### Routes of Administration and Dosing

Viral vectors, such as AAV vectors and others described herein, containing a transgene encoding an interfering RNA construct described herein may be administered to a patient (e.g., a human patient) by a variety of routes of administration. The route of administration may vary, for example, with the onset and severity of disease, and may include, e.g., intravenous, intrathecal, intracerebroventricular, intraparenchymal, intracisternal, intradermal, transdermal, parenteral, intramuscular, intranasal, subcutaneous, percutaneous, intratracheal, intraperitoneal, intraarterial, intravascular, inhalation, perfusion, lavage, and oral administration. Intravascular administration includes delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Treatment regimens may vary, and often depend on disease severity and the age, weight, and sex of the patient. Treatment may include administration of vectors (e.g., viral vectors) or other agents described herein as useful for the introduction of a transgene into a target cell in various unit doses. Each unit dose will ordinarily contain a predetermined-quantity of the therapeutic composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Development and Evaluation of Adeno-Associated Viral Vectors Encoding miRNA Constructs for the Treatment of Disorders Associated with RNA Dominance

### Objective

This example describes a series of experiments conducted in order to characterize the development and evaluation of rAAV vectors encoding miRNA constructs against human DMPK and murine HSA^{LR}, which is expressed in mouse models of the RNA dominance disorder, myotonic dystrophy. Myotonic dystrophy (DM) is caused by expansion of a microsatellite repeat that leads to expression of a toxic expanded repeat mRNA. Another RNA dominance disorder, facioscapulohumeral muscular dystrophy (FSHD), is caused by contraction of a D4Z4 macrosatellite repeat that leads to expression of DUX4, a toxic protein in adult muscle. The goal of the experiments described in this example is to develop AAV vectors encoding miRNA constructs that target muscle DM and FSHD-related mRNAs, so as to prevent muscle dysfunction and loss in individuals with disease.

### Materials and Methods

To develop rAAV-RNAi therapy for myotonic dystrophy type 1 (DM1), a human skeletal actin gene (HSA) directed interfering RNA hairpin was designed and produced so that its efficacy could be tested in the HSA^{LR} mouse model of DM1. The HSA^{LR} mouse was produced with insertion of an expanded CTG repeat in the 3′ UTR of the HSA gene, a similar genetic context to the disease-causing repeat expansion in the DMPK gene in humans. The HSA^{LR} mouse displays many of the genetic and phenotypic changes associated with DM1 in skeletal muscle, including myotonia, splicing changes in a variety of mRNAs, and nuclear inclusions (foci). Among the aims of this study was to translate the approach of reducing expression of aberrant HSA^{LR} RNA transcripts containing expanded CUG repeat regions to develop a paradigm for suppressing the human disease target gene, DMPK.

To this end, RNAi expression cassettes with 19-22 bp target recognition sequences were tested for a variety of applications. The RNAi hairpins were based on miR30a endogenous sequence. The single-stranded rAAV genomes were packaged in an rAAV6 capsid for targeting muscle. rAAV6 HSA RNAi constructs were delivered by intravenous injection (IV) previously into the tail vein of HSA^{LR} mice at 4 weeks of age (n=7-9).

### Results

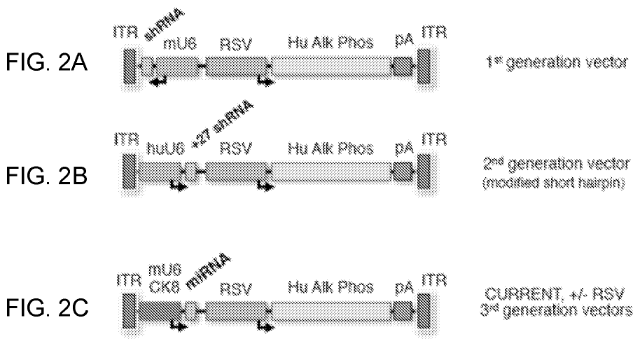

As shown in FIGS. 2A-2C, three generations of rAAV-RNAi vectors were developed to target several genes for testing and development of therapeutic RNAi. The rAAV plasmid pARAP4 includes the human alkaline phosphatase reporter gene (Hu Alk Phos) reporter gene, expressed from the Rous Sarcoma Virus (RSV) promoter, and the SV40 polyadenylation sequence, pA. The inverted terminal repeats (ITRs) originate from rAAV2 and genomes are packaged in rAAV6 capsids. The newest vector modification removes the RSV promoter sequence to prevent Hu Alk Phos expression that limited rAAV-RNAi efficacy at higher doses due muscle cell toxicity.

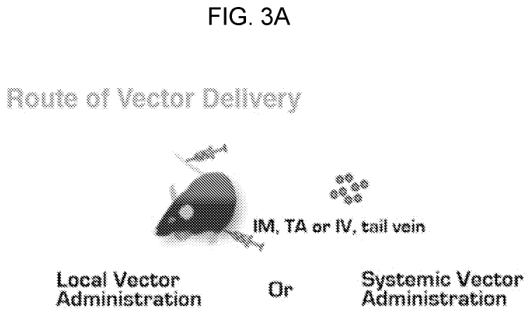

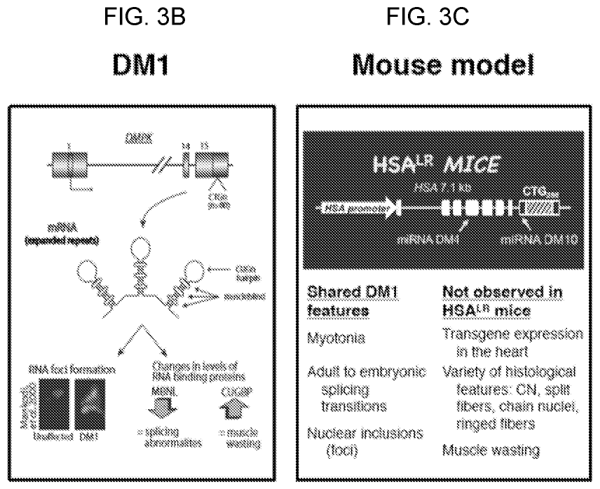

As show in FIGS. 3A-3C, HSA^{LR} mice show characteristics of muscular dystrophy resembling DM in humans. The HSA^{LR} transgene is derived from insertion of a (CTG)₂₅₀ repeat in the 3′ UTR of the HSA gene. When the transgene is expressed in mouse skeletal muscle, myotonic discharges are evident, splicing alterations occur in a variety of mRNAs, and nuclear foci containing the expanded transgenic mRNA and splicing factors are present.

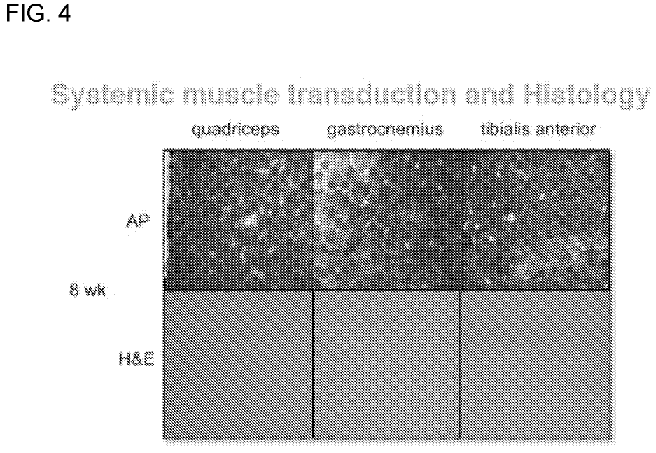

As shown in FIG. 4, HSA^{LR} mice were transduced with rAAV6 HSA miR DM10. Human placental alkaline phosphatase (AP) staining indicates presence of the viral genome with active reporter gene expression, and H&E staining of cryosections from treated mice is also shown. Timepoint, 8 weeks post-injection, 4 week-old HSA^{LR} mice.

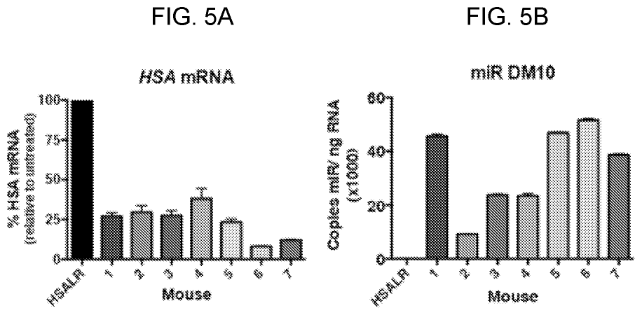

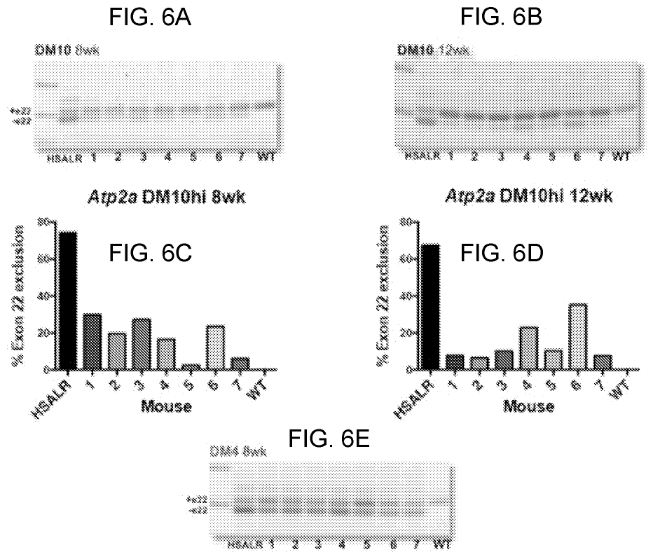

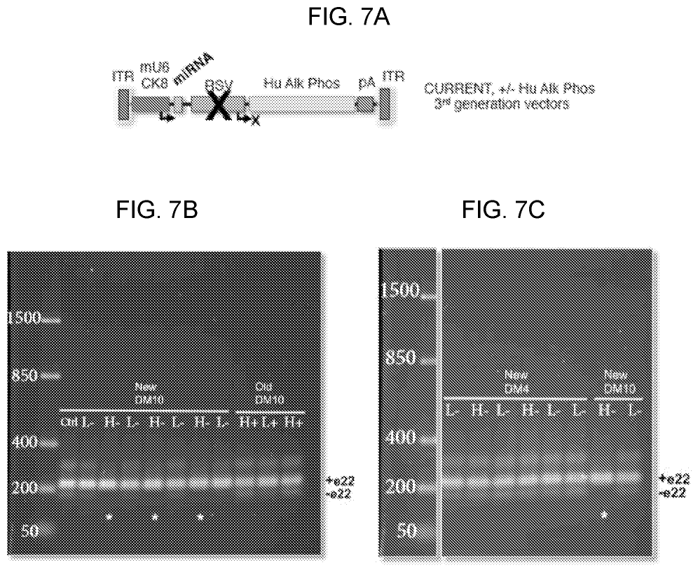

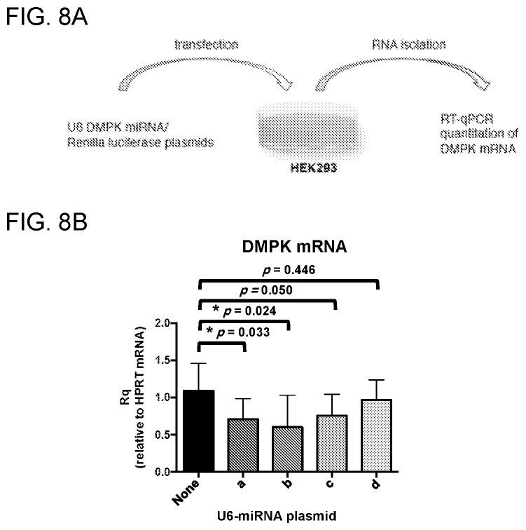

As shown in FIGS. 5A and 5B, FIGS. 6A-6E, and FIGS. 7A-7C, rAAV-RNAi vectors encoding miRNA constructs complimentary to HSA^{LR} transcripts reduced the expression of pathologic HSA^{LR} RNA and effectuated the release of RNA-binding splicing factors that would otherwise be sequestered by the CUG repeat expansion, as evidenced by the restoration of correct splicing of SERCA1 mRNA (FIGS. 6C and 6D). rAAV6 HSA miR DM10 systemic injection improves splicing of SERCA1 and CLCN1 in the tibialis anterior (TA) muscle. In contrast, a different RNAi hairpin, miR DM4, was not as effective at reversing these splicing defects.

### Selection of DMPK Regions for RNAi

Selection of DMPK RNA target sequences for incorporation into miRNA-based hairpins was done using guidelines for siRNA design. Candidate target sequences were eliminated based on predicted seed sequence matches at other loci or in alternatively spliced regions. Additional screening included analysis using Bowtie for short sequence alignment to the human genome and extensive BLAST searches. Exemplary interfering RNA constructs against human DMPK are shown in Table 4, above, and are represented graphically in FIG. 1.

### Conclusions

As demonstrated in these experiments, local injection of vectors lacking the Hu Alk Phos promoter in muscle improves the splicing-related phenotype of the HSA^{LR} mice, as measured by the increase in amount of SERCA1 mRNA adult splicing product. Splicing for SERCA1 mRNA was approximately 95% corrected and demonstrates that improvements in technology have the potential to increase the efficacy of this approach.

Additionally, these results demonstrate that rAAV6-mediated delivery of the HSA hairpins improves many molecular and phenotypic characteristics of DM1 modeled in the HSA^{LR} mouse, including myotonia, disease-related splicing changes, and sequestration of splicing factors. Moreover, using the compositions and methods described herein, vectors carrying U6-expressed DMPK miRNAs can reduce endogenous DMPK mRNA in HEK293 cells following transfection. These data indicate the utility for an RNAi-mediated gene therapy to treat DM1.

rAAV-mediated therapy is effective for spinal muscular atrophy and is progressing in clinical trials for Duchenne muscular dystrophy. Studies in non-human primates demonstrate that AAV can transduce muscle efficiently with regional limb delivery and persist for as long as 10 years. These studies support development of rAAV-mediated RNAi gene therapy for the treatment of dominant muscle diseases in humans, such as myotonic dystrophy type I, among others described herein.

### Example 2. Treatment of Myotonic Dystrophy in a Human Patient by Administration of a Viral Vector Encoding a miRNA Against DMPK

Using the compositions and methods described herein, a physician of skill in the art may administer to a patient having myotonic dystrophy type I a viral vector encoding a miRNA that anneals to, and reduces the expression of, mutant DMPK RNA transcripts containing expanded CUG repeat regions. The vector may be an AAV vector, such as a pseudotyped AAV2/8 or AAV2/9 vector. The vector may be administered by way of one or more routes of administration described herein, such as by intravenous, intrathecal, intracerebroventricular, intraparenchymal, intracisternal, intramuscular, or subcutaneous injection. The encoded miRNA may be a miRNA characterized herein, such as a miRNA having the nucleic acid sequence of any one of SEQ ID Nos: 40-161.

Following administration of the vector, the physician may monitor the progression of the disorder and the efficacy of the treatment by assessing, for example, the concentration of correctly spliced mRNA transcripts encoding SERCA1, CLCN1, and/or ZASP, using an RNA detection technique described herein. The physician may also monitor the concentration of functional SERCA1, CLCN1, and/or ZASP protein product resulting from the correctly spliced transcripts. Additionally or alternatively, the physician may monitor the concentration of mutant DMPK RNA transcripts expressed by the patient, particularly, DMPK transcripts having from about 50 to about 4,000, or more, CUG trinucleotide repeats. A finding that (i) the concentration of correctly spliced SERCA1, CLCN1, and/or ZASP mRNA transcripts has increased, (ii) the concentration of functional SERCA1, CLCN1, and/or ZASP protein products resulting from translation of the correctly spliced mRNA transcripts has increased, and/or (iii) the concentration of mutant DMPK RNA transcripts harboring expanded CUG trinucleotide repeat regions has decrease may serve as an indicator that the patient has been successfully treated. The physician may also monitor the progression of one or more symptoms of the disease, such as myotonia, muscle stiffness, disabling distal weakness, weakness in the face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. In children, symptoms may also include developmental delays, learning problems, language and speech difficulties, and personality development challenges. A finding that one or more, or all, of the foregoing symptoms has been ameliorated may also serve as a clinical indicator of successful treatment.

### Example 3. Treatment of Myotonic Dystrophy in a Human Patient by Administration of an siRNA Oligonucleotide Against DMPK

Using the compositions and methods described herein, a physician of skill in the art may administer to a patient having myotonic dystrophy type I an siRNA oligonucleotide that anneals to, and reduces the expression of, mutant DMPK RNA transcripts containing expanded CUG repeat regions. The oligonucleotide may have, for example, the nucleic acid sequence of any one of SEQ ID NOs: 3-39.

Following administration of the oligonucleotide, the physician may monitor the progression of the disorder and the efficacy of the treatment by assessing, for example, the concentration of correctly spliced mRNA transcripts encoding SERCA1, CLCN1, and/or ZASP, using an RNA detection technique described herein. The physician may also monitor the concentration of functional SERCA1, CLCN1, and/or ZASP protein product resulting from the correctly spliced transcripts. Additionally or alternatively, the physician may monitor the concentration of mutant DMPK RNA transcripts expressed by the patient, particularly, DMPK transcripts having from about 50 to about 4,000, or more, CUG trinucleotide repeats. A finding that (i) the concentration of correctly spliced SERCA1, CLCN1, and/or ZASP mRNA transcripts has increased, (ii) the concentration of functional SERCA1, CLCN1, and/or ZASP protein products resulting from translation of the correctly spliced mRNA transcripts has increased, and/or (iii) the concentration of mutant DMPK RNA transcripts harboring expanded CUG trinucleotide repeat regions has decrease may serve as an indicator that the patient has been successfully treated. The physician may also monitor the progression of one or more symptoms of the disease, such as myotonia, muscle stiffness, disabling distal weakness, weakness in the face and jaw muscles, difficulty in swallowing, drooping of the eyelids (ptosis), weakness of neck muscles, weakness in arm and leg muscles, persistent muscle pain, hypersomnia, muscle wasting, dysphagia, respiratory insufficiency, irregular heartbeat, heart muscle damage, apathy, insulin resistance, and cataracts. In children, symptoms may also include developmental delays, learning problems, language and speech difficulties, and personality development challenges. A finding that one or more, or all, of the foregoing symptoms has been ameliorated may also serve as a clinical indicator of successful treatment.

### Example 4. Ability of Anti-DMPK siRNA Molecules to Suppress DMPK1 Expression in Cultured HEK293 Cells

This example describes the results of experiments conducted in order to evaluate the ability of anti-DMPK siRNA molecules, such as various siRNA molecules described in Table 4 herein, to attenuate the expression of DMPK1 mRNA in cultured human cells. For purposes of comparison, in addition to testing siRNA molecules described in Table 4, above, a scrambled siRNA molecule and commercially available anti-DMPK siRNA molecules were tested as well.

To conduct these experiments, HEK293 cells (2×10⁵ cells/well) were transfected in triplicate with either 5 μM of a candidate anti-DMPK siRNA molecule (such as an siRNA molecule described in Table 4, above) or 5 μM scrambled negative siRNA control (Silencer® Select siRNA, Ambion by Life Technologies) using 1 μL Lipofectamine™ RNAiMAX (Thermo Fisher Scientific). Mock transfections of cells treated only with 1 μL Lipofectamine™ RNAiMAX were included for normalization. RNA was harvested after 48 hours using the RNeasy Plus Mini Kit (Qiagen). cDNA generated was subsequently generated using SuperScript™ III Reverse Transcriptase (Thermo Fisher Scientific) using 150 ng of RNA per sample. qPCR was performed to detect DMPK1 knockdown. qPCR experiments were set up in triplicate using the TaqMan™ Fast Advanced Master Mix, and reactions were performed using a QuantStudio 3 RT-PCR instrument (Thermo Fisher Scientific). DMPK1 expression values were normalized to GAPDH (TaqMan™ Gene expression assay ID Hs02786624_g1) using QuantStudio 3 software.

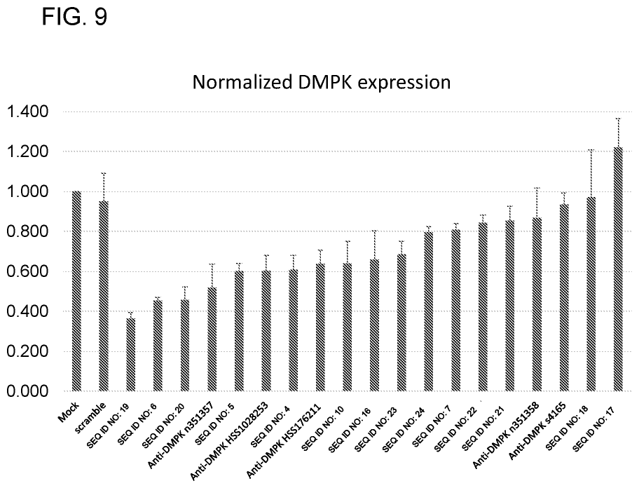

The results of these experiments are shown in FIG. 9. As evidenced by this figure, various siRNA molecules described in Table 4, above, are capable of downregulating DMPK1 expression in live human cells. The data shown graphically in FIG. 9 are provided in numerical form in Table 6, below.

TABLE 6
Suppression of DMPK1 expression in HEK293
cells by siRNA molecules in FIG. 9

| siRNA Molecule | Normalized DMPK1 Expression |
|---|---|
| SEQ ID NO: 19 | 36.535% |
| SEQ ID NO: 6 | 45.575% |
| SEQ ID NO: 20 | 45.748% |
| Anti-DMPK n351357 | 51.946% |
| SEQ ID NO: 5 | 60.258% |
| Anti-DMPK HSS1028253 | 60.498% |
| SEQ ID NO: 4 | 60.926% |
| Anti-DMPK HSS176211 | 63.934% |
| SEQ ID NO: 10 | 64.104% |
| SEQ ID NO: 16 | 66.067% |
| SEQ ID NO: 23 | 68.659% |
| SEQ ID NO: 24 | 79.758% |
| SEQ ID NO: 7 | 80.929% |
| SEQ ID NO: 22 | 84.449% |
| SEQ ID NO: 21 | 85.495% |
| Anti-DMPK n351358 | 86.927% |
| Anti-DMPK s4165 | 93.519% |
| SEQ ID NO: 18 | 97.217% |
| SEQ ID NO: 17 | 122.154% |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. A viral vector comprising one or more transgenes, each encoding an interfering ribonucleic acid (RNA) of at least 17 nucleotides in length, wherein each interfering RNA comprises a portion that anneals to an endogenous RNA transcript comprising an expanded repeat region, and wherein the portion of each interfering RNA anneals to a segment of the endogenous RNA transcript that does not overlap with the expanded repeat region.

2. The viral vector of claim 1, wherein the endogenous RNA transcript encodes human dystrophia myotonica protein kinase (DMPK).

3. The viral vector of claim 2, wherein the expanded repeat region comprises 50 or more CUG trinucleotide repeats.

4. (canceled)

5. The viral vector of claim 2, wherein the vector further comprises a transgene encoding a human DMPK RNA transcript that does not anneal to the interfering RNA.

6. The viral vector of claim 5, wherein the human DMPK RNA transcript is less than 85% complementary to the interfering RNA.

7. The viral vector of claim 2, wherein the endogenous RNA transcript comprises a portion having at least 85% sequence identity to the nucleic acid sequence of SEQ ID NO: 1 or SEQ ID NO: 2.

8-11. (canceled)

12. The viral vector of claim 2, wherein the portion of each interfering RNA has a nucleic acid sequence that is least 85% complementary to the nucleic acid sequence of a segment within any one of exons 1-15 of human DMPK, any one of introns 1-14 of human DMPK, an exon-intron boundary within human DMPK, or within the 5′ untranslated region (UTR) or 3′ UTR of human DMPK.

13-33. (canceled)

34. The viral vector of claim 2, wherein the portion of each interfering RNA anneals to a segment of the endogenous RNA transcript having the nucleic acid sequence of any one of SEQ ID NOs: 3-39.

35-38. (canceled)

39. The viral vector of claim 2, wherein the interfering RNA comprises a portion having at least 85% sequence identity to the nucleic acid sequence of any one of SEQ ID NOs: 3-161.

40-42. (canceled)

43. The viral vector of claim 1, wherein the endogenous RNA transcript comprises human chromosome 9 open reading frame 72 (C9ORF72) and an expanded repeat region.

44-49. (canceled)

50. The viral vector of claim 43, wherein the portion of each interfering RNA has a nucleic acid sequence that is at least 85% complementary to the nucleic acid sequence of a segment within human C9ORF72.

51-60. (canceled)

61. The viral vector of claim 1, wherein the interfering RNA is a short interfering RNA (siRNA), a short hairpin RNA (shRNA), or a micro RNA (miRNA).

62-64. (canceled)

65. The viral vector of claim 1, wherein the interfering RNA is operably linked to a promoter that induces expression of the interfering RNA in a muscle cell or neuron.

66. The viral vector of claim 65, wherein the promoter is a desmin promoter, a phosphoglycerate kinase (PGK) promoter, a muscle creatine kinase promoter, a myosin light chain promoter, a myosin heavy chain promoter, a cardiac troponin C promoter, a troponin I promoter, a myoD gene family promoter, an actin alpha promoter, an actin beta promoter, an actin gamma promoter, or a promoter within intron 1 of ocular paired like homeodomain 3 (PITX3).

67. (canceled)

68. The viral vector of claim 1, wherein the viral vector is an adeno-associated virus (AAV), optionally wherein the AAV is an AAV1, AAV2, AAV3, AAV4, AAV5, AAV6, AAV7, AAV8, AAV9, AAVrh10, or AAVrh74 serotype.

69. (canceled)

70. The viral vector of claim 68, wherein the viral vector is AAV2/8 or AAV2/9.

71-74. (canceled)

75. A nucleic acid encoding or comprising an interfering RNA, wherein the interfering RNA comprises a portion having at least 85% sequence identity to the nucleic acid sequence of any one of SEQ ID NOs: 3-161.

76-116. (canceled)

117. A method of reducing the occurrence of spliceopathy in a human patient in need thereof, the method comprising administering to the patient a therapeutically effective amount of the vector of claim 1.

118. The method of claim 117, wherein the patient has myotonic dystrophy or amyotrophic lateral sclerosis.

119-124. (canceled)

125. A method of treating a disorder characterized by nuclear retention of RNA comprising an expanded repeat region in a human patient in need thereof, the method comprising administering to the patient a therapeutically effective amount of the vector of claim 1.

126-130. (canceled)


SEQUENCE LISTING (166 sequences)

SEQ ID NO: 1 - RNA, 2888 nt, Homo sapiens
ggcuggaccaagggguggggagaaggggaggaggccucggccggccgcagagagaaguggccagagaggcccaggggacagccagggacaggcagacaugcagccagggcuccagggccuggacaggggcugccaggcccugugacaggaggaccccgagcccccggcccggggaggggccauggugcugccuguccaacaugucagccgaggugcggcugaggcggcuccagcagcugguguuggacccgggcuuccuggggcuggagccccugcucgaccuucuccugggcguccaccaggagcugggcgccuccgaacuggcccaggacaaguacguggccgacuucuugcagugggccccaaauccaggguuuuccaaagugugguucaagaaccaccugcaucugaaucuagagcggagcccaucguggugaggcuuaaggagguccgacugcagagggacgacuucgagauucugaaggugaucggacgcggggcguucagcgagguagcgguagugaagaugaagcagacgggccagguguaugccaugaagaucaugaacaagugggacaugcugaagaggggcgaggugucgugcuuccgugaggagagggacguguuggugaauggggaccagcgguggaucacgcagcugcacuucgccuuccaggaugagaacuaccuguaccuggucauggaguauuacgugggcggggaccugcugacacugcugagcaaguuuggggagcggauuccggccgagauggcgcgcuucuaccuggcggagauugucauggccauagacucggugcaccggcuuggcuacgugcacagggacaucaaacccgacaacauccugcuggaccgcuguggccacauccgccuggccgacuucggcucuugccucaagcugcgggcagauggaacggugcggucgcugguggcugugggcaccccagacuaccugucccccgagauccugcaggcugugggcggugggccugggacaggcagcuacgggcccgagugugacuggugggcgcuggguguauucgccuaugaaauguucuaugggcagacgcccuucuacgcggauuccacggcggagaccuauggcaagaucguccacuacaaggagcaccucucucugccgcugguggacgaaggggucccugaggaggcucgagacuucauucagcgguugcugugucccccggagacacggcugggccgggguggagcaggcgacuuccggacacaucccuucuucuuuggccucgacugggauggucuccgggacagcgugccccccuuuacaccggauuucgaaggugccaccgacacaugcaacuucgacuugguggaggacgggcucacugccauggugagcgggggcggggagacacugucggacauucgggaaggugcgccgcuagggguccaccugccuuuugugggcuacuccuacuccugcauggcccucagggacagugaggucccaggccccacacccauggaacuggaggccgagcagcugcuugagccacacgugcaagcgcccagccuggagcccucgguguccccacaggaugaaacagcugaaguggcaguuccagcggcugucccugcggcagaggcugaggccgaggugacgcugcgggagcuccaggaagcccuggaggaggaggugcucacccggcagagccugagccgggagauggaggccauccgcacggacaaccagaacuucgccagucaacuacgcgaggcagaggcucggaaccgggaccuagaggcacacguccggcaguugcaggagcggauggaguugcugcaggcagagggagccacagcugucacggggguccccaguccccgggccacggauccaccuucccaucuagauggccccacggccguggcugugggccagugcccgcugguggggccaggccccaugcaccgccgccaccugcugcucccugccagggucccuaggccuggccuaucggaggcgcuuucccugcuccuguucgccguuguucugucucgugccgccgcccugggcugcauuggguugguggcccacgccggccaacucaccgcagucuggcgccgcccaggagccgcccgcgcucccugaacccuagaacugucuucgacucaggggccccguuggaagacugagugcccggggcacggcacagaagccgcgcccaccgccugccaguucacaaccgcuccgagcgugggucuccgcccagcaccaguccugugauccgggcccgcccccuagcggccggggagggaggggccggguccgcggccggcgaacggggcucgaaggguccuuguagccgggaaugcugcugcugcugcuggggggaucacagaccauuucuuucuuucggccaggcugaggcccugacguggaugggcaaacugcaggccugggaaggcagcaagccgggccguccguguuccauccuccacgcacccccaccuaucguugguucgcaaagugcaaagcuuucuugugcaugacgcccugcucuggggagcgucuggcgcgaucucugccugcuuacccgggaaauuugcuuuugccaaacccgcuuuuucggggaucccgcgccccccuccucacuugcgcugcucucggagccccagccggcuccgcccgcuucggcgguuuggauauuuauugaccucguccuccgacucgcugacaggcuacaggacccccaacaaccccaauccacguuuuggaugcacugagaccccgacauuccucgguauuuauugucuguccccaccuaggacccccacccccgacccucgcgaauaaaaggcccuccaucugcccaaaaaaaaaaaaaaaa

SEQ ID NO: 2 - RNA, 2768 nt, Homo sapiens
gggacagccagggacaggcagacaugcagccagggcuccagggccuggacaggggcugccaggcccugugacaggaggaccccgagcccccggcccggggaggggccauggugcugccuguccaacaugucagccgaggugcggcugaggcggcuccagcagcugguguuggacccgggcuuccuggggcuggagccccugcucgaccuucuccugggcguccaccaggagcugggcgccuccgaacuggcccaggacaaguacguggccgacuucuugcagugggcggagcccaucguggugaggcuuaaggagguccgacugcagagggacgacuucgagauucugaaggugaucggacgcggggcguucagcgagguagcgguagugaagaugaagcagacgggccagguguaugccaugaagaucaugaacaagugggacaugcugaagaggggcgaggugucgugcuuccgugaggagagggacguguuggugaauggggaccggcgguggaucacgcagcugcacuucgccuuccaggaugagaacuaccuguaccuggucauggaguauuacgugggcggggaccugcugacacugcugagcaaguuuggggagcggauuccggccgagauggcgcgcuucuaccuggcggagauugucauggccauagacucggugcaccggcuuggcuacgugcacagggacaucaaacccgacaacauccugcuggaccgcuguggccacauccgccuggccgacuucggcucuugccucaagcugcgggcagauggaacggugcggucgcugguggcugugggcaccccagacuaccugucccccgagauccugcaggcugugggcggugggccugggacaggcagcuacgggcccgagugugacuggugggcgcuggguguauucgccuaugaaauguucuaugggcagacgcccuucuacgcggauuccacggcggagaccuauggcaagaucguccacuacaaggagcaccucucucugccgcugguggacgaaggggucccugaggaggcucgagacuucauucagcgguugcugugucccccggagacacggcugggccgggguggagcaggcgacuuccggacacaucccuucuucuuuggccucgacugggauggucuccgggacagcgugccccccuuuacaccggauuucgaaggugccaccgacacaugcaacuucgacuugguggaggacgggcucacugccauggugagcgggggcggggagacacugucggacauucgggaaggugcgccgcuagggguccaccugccuuuugugggcuacuccuacuccugcauggcccucagggacagugaggucccaggccccacacccauggaacuggaggccgagcagcugcuugagccacacgugcaagcgcccagccuggagcccucgguguccccacaggaugaaacagcugaaguggcaguuccagcggcugucccugcggcagaggcugaggccgaggugacgcugcgggagcuccaggaagcccuggaggaggaggugcucacccggcagagccugagccgggagauggaggccauccgcacggacaaccagaacuucgccagucaacuacgcgaggcagaggcucggaaccgggaccuagaggcacacguccggcaguugcaggagcggauggaguugcugcaggcagagggagccacagcugucacggggguccccaguccccgggccacggauccaccuucccaucuagauggccccccggccguggcugugggccagugcccgcugguggggccaggccccaugcaccgccgccaccugcugcucccugccagggucccuaggccuggccuaucggaggcgcuuucccugcuccuguucgccguuguucugucucgugccgccgcccugggcugcauuggguugguggcccacgccggccaacucaccgcagucuggcgccgcccaggagccgcccgcgcucccugaacccuagaacugucuucgacuccggggccccguuggaagacugagugcccggggcacggcacagaagccgcgcccaccgccugccaguucacaaccgcuccgagcgugggucuccgcccagcuccaguccugugauccgggcccgcccccuagcggccggggagggaggggccggguccgcggccggcgaacggggcucgaaggguccuuguagccgggaaugcugcugcugcugcuggggggaucacagaccauuucuuucuuucggccaggcugaggcccugacguggaugggcaaacugcaggccugggaaggcagcaagccgggccguccguguuccauccuccacgcacccccaccuaucguugguucgcaaagugcaaagcuuucuugugcaugacgcccugcucuggggagcgucuggcgcgaucucugccugcuuacucgggaaauuugcuuuugccaaacccgcuuuuucggggaucccgcgccccccuccucacuugcgcugcucucggagccccagccggcuccgcccgcuucggcgguuuggauauuuauugaccucguccuccgacucgcugacaggcuacaggacccccaacaaccccaauccacguuuuggaugcacugagaccccgacauuccucgguauuuauugucuguccccaccuaggacccccacccccgacccucgcgaauaaaaggcccuccaucugcccaaaaaaaaaaaaaaaaaaaaaaaaaaaaa

SEQ ID NO: 3 - RNA, 18 nt, Artificial Sequence
  Synthetic Construct
cgccagucaacuacgcga

SEQ ID NO: 4 - RNA, 19 nt, Artificial Sequence
  Synthetic Construct
ccugcuccuguucgccguu

SEQ ID NO: 5 - RNA, 19 nt, Artificial Sequence
  Synthetic Construct
cccgacauuccucgguauu

SEQ ID NO: 6 - RNA, 19 nt, Artificial Sequence
  Synthetic Construct
ccuagaacugucuucgacu

SEQ ID NO: 7 - RNA, 19 nt, Artificial Sequence
  Synthetic Construct
ccuaucguugguucgcaaa

SEQ ID NO: 8 - RNA, 19 nt, Artificial Sequence
  Synthetic Construct
gcuggguguauucgccuau

SEQ ID NO: 9 - RNA, 19 nt, Artificial Sequence
  Synthetic Construct
ggacaucaaacccgacaac

SEQ ID NO: 10 - RNA, 19 nt, Artificial Sequence
  Synthetic Construct
gcgggcagauggaacggug

SEQ ID NO: 11 - RNA, 19 nt, Artificial Sequence
  Synthetic Construct
ggccuaucggaggcgcuuu

SEQ ID NO: 12 - RNA, 19 nt, Artificial Sequence
  Synthetic Construct
cccuagaacugucuucgac

SEQ ID NO: 13 - RNA, 19 nt, Artificial Sequence
  Synthetic Construct
ggacaaccagaacuucgcc

SEQ ID NO: 14 - RNA, 19 nt, Artificial Sequence
  Synthetic Construct
cgcuggguguauucgccua

SEQ ID NO: 15 - RNA, 19 nt, Artificial Sequence
  Synthetic Construct
caccuaucguugguucgca

SEQ ID NO: 16 - RNA, 19 nt, Artificial Sequence
  Synthetic Construct
ggcgcuggguguauucgcc

SEQ ID NO: 17 - RNA, 19 nt, Artificial Sequence
  Synthetic Construct
cgcccuucuacgcggauuc

SEQ ID NO: 18 - RNA, 19 nt, Artificial Sequence
  Synthetic Construct
cgaaggugccaccgacaca

SEQ ID NO: 19 - RNA, 19 nt, Artificial Sequence
  Synthetic Construct
ccauuucuuucuuucggcc

SEQ ID NO: 20 - RNA, 19 nt, Artificial Sequence
  Synthetic Construct
gccguuguucugucucgug

SEQ ID NO: 21 - RNA, 19 nt, Artificial Sequence
  Synthetic Construct
gcuccuguucgccguuguu

SEQ ID NO: 22 - RNA, 19 nt, Artificial Sequence
  Synthetic Construct
gccaguucacaaccgcucc

SEQ ID NO: 23 - RNA, 19 nt, Artificial Sequence
  Synthetic Construct
ccaguucacaaccgcuccg

SEQ ID NO: 24 - RNA, 19 nt, Artificial Sequence
  Synthetic Construct
gguccuuguagccgggaau

SEQ ID NO: 25 - RNA, 19 nt, Artificial Sequence
  Synthetic Construct
ccacagucaacuacgcgag

SEQ ID NO: 26 - RNA, 19 nt, Artificial Sequence
  Synthetic Construct
cgaccucagguagcgguag

SEQ ID NO: 27 - RNA, 19 nt, Artificial Sequence
  Synthetic Construct
cgggaaggugcgccgcuag

SEQ ID NO: 28 - RNA, 19 nt, Artificial Sequence
  Synthetic Construct
gggauucgaggcgugcgag

SEQ ID NO: 29 - RNA, 19 nt, Artificial Sequence
  Synthetic Construct
ggcuuaaggagguccgacu

SEQ ID NO: 30 - RNA, 19 nt, Artificial Sequence
  Synthetic Construct
cccuacgucuuugcgacuu

SEQ ID NO: 31 - RNA, 19 nt, Artificial Sequence
  Synthetic Construct
cgacugcagagggacgacu

SEQ ID NO: 32 - RNA, 19 nt, Artificial Sequence
  Synthetic Construct
ccaaugacgaguucggacg

SEQ ID NO: 33 - RNA, 19 nt, Artificial Sequence
  Synthetic Construct
gguccaggugcggucgcug

SEQ ID NO: 34 - RNA, 19 nt, Artificial Sequence
  Synthetic Construct
gcaggagacacugucggac

SEQ ID NO: 35 - RNA, 19 nt, Artificial Sequence
  Synthetic Construct
cccacgucugggccguuac

SEQ ID NO: 36 - RNA, 19 nt, Artificial Sequence
  Synthetic Construct
gcaccggcuuggcuacgug

SEQ ID NO: 37 - RNA, 19 nt, Artificial Sequence
  Synthetic Construct
gcuugauucugaaccgcug

SEQ ID NO: 38 - RNA, 19 nt, Artificial Sequence
  Synthetic Construct
gcuuaaggagguccgacug

SEQ ID NO: 39 - RNA, 19 nt, Artificial Sequence
  Synthetic Construct
cgugcugucacuggacgag

SEQ ID NO: 40 - RNA, 21 nt, Artificial Sequence
  Synthetic Construct
agcgccagucaacuacgcgag

SEQ ID NO: 41 - RNA, 21 nt, Artificial Sequence
  Synthetic Construct
ccccugcuccuguucgccguu

SEQ ID NO: 42 - RNA, 21 nt, Artificial Sequence
  Synthetic Construct
agcccgacauuccucgguauu

SEQ ID NO: 43 - RNA, 21 nt, Artificial Sequence
  Synthetic Construct
acccuagaacugucuucgauu

SEQ ID NO: 44 - RNA, 21 nt, Artificial Sequence
  Synthetic Construct
acccuaucguugguucgcaaa

SEQ ID NO: 45 - RNA, 21 nt, Artificial Sequence
  Synthetic Construct
acgcuggguguauucgccuau

SEQ ID NO: 46 - RNA, 21 nt, Artificial Sequence
  Synthetic Construct
cgggacaucaaacccgacaau

SEQ ID NO: 47 - RNA, 21 nt, Artificial Sequence
  Synthetic Construct
acgcgggcagauggaacggug

SEQ ID NO: 48 - RNA, 21 nt, Artificial Sequence
  Synthetic Construct
acggccuaucggaggcgcuuu

SEQ ID NO: 49 - RNA, 21 nt, Artificial Sequence
  Synthetic Construct
agcccuagaacugucuucgau

SEQ ID NO: 50 - RNA, 21 nt, Artificial Sequence
  Synthetic Construct
acggacaaccagaacuucguu

SEQ ID NO: 51 - RNA, 21 nt, Artificial Sequence
  Synthetic Construct
agcgcuggguguauucgcuua

SEQ ID NO: 52 - RNA, 21 nt, Artificial Sequence
  Synthetic Construct
accaccuaucguugguucgua

SEQ ID NO: 53 - RNA, 21 nt, Artificial Sequence
  Synthetic Construct
cgggcgcuggguguauucguu

SEQ ID NO: 54 - RNA, 21 nt, Artificial Sequence
  Synthetic Construct
accgcccuucuacgcggauuu

SEQ ID NO: 55 - RNA, 21 nt, Artificial Sequence
  Synthetic Construct
cgcgaaggugccaccgacaua

SEQ ID NO: 56 - RNA, 21 nt, Artificial Sequence
  Synthetic Construct
acccauuucuuucuuucgguu

SEQ ID NO: 57 - RNA, 21 nt, Artificial Sequence
  Synthetic Construct
acgccguuguucugucucgug

SEQ ID NO: 58 - RNA, 21 nt, Artificial Sequence
  Synthetic Construct
acgcuccuguucgccguuguu

SEQ ID NO: 59 - RNA, 21 nt, Artificial Sequence
  Synthetic Construct
acgccaguucacaaccgcuuu

SEQ ID NO: 60 - RNA, 21 nt, Artificial Sequence
  Synthetic Construct
cgccaguucacaaccgcuuug

SEQ ID NO: 61 - RNA, 21 nt, Artificial Sequence
  Synthetic Construct
cggguccuuguagccgggaau

SEQ ID NO: 62 - RNA, 21 nt, Artificial Sequence
  Synthetic Construct
acccacagucaacuacgcgag

SEQ ID NO: 63 - RNA, 21 nt, Artificial Sequence
  Synthetic Construct
accgaccucagguagcgguag

SEQ ID NO: 64 - RNA, 21 nt, Artificial Sequence
  Synthetic Construct
cgcgggaaggugcgccgcuag

SEQ ID NO: 65 - RNA, 21 nt, Artificial Sequence
  Synthetic Construct
acgggauucgaggcgugcgag

SEQ ID NO: 66 - RNA, 21 nt, Artificial Sequence
  Synthetic Construct
acggcuuaaggagguccgauu

SEQ ID NO: 67 - RNA, 21 nt, Artificial Sequence
  Synthetic Construct
agcccuacgucuuugcgauuu

SEQ ID NO: 68 - RNA, 21 nt, Artificial Sequence
  Synthetic Construct
cccgacugcagagggacgauu

SEQ ID NO: 69 - RNA, 21 nt, Artificial Sequence
  Synthetic Construct
cgccaaugacgaguucggaug

SEQ ID NO: 70 - RNA, 21 nt, Artificial Sequence
  Synthetic Construct
cggguccaggugcggucguug

SEQ ID NO: 71 - RNA, 21 nt, Artificial Sequence
  Synthetic Construct
acgcaggagacacugucggau

SEQ ID NO: 72 - RNA, 21 nt, Artificial Sequence
  Synthetic Construct
cgcccacgucugggccguuau

SEQ ID NO: 73 - RNA, 21 nt, Artificial Sequence
  Synthetic Construct
acgcaccggcuuggcuacgug

SEQ ID NO: 74 - RNA, 21 nt, Artificial Sequence
  Synthetic Construct
acgcuugauucugaaccguug

SEQ ID NO: 75 - RNA, 21 nt, Artificial Sequence
  Synthetic Construct
cggcuuaaggagguccgauug

SEQ ID NO: 76 - RNA, 21 nt, Artificial Sequence
  Synthetic Construct
accgugcugucacuggacgag

SEQ ID NO: 77 - RNA, 21 nt, Artificial Sequence
  Synthetic Construct
uucgcguaguugacuggcgcc

SEQ ID NO: 78 - RNA, 21 nt, Artificial Sequence
  Synthetic Construct
aacggcgaacaggagcagggc

SEQ ID NO: 79 - RNA, 21 nt, Artificial Sequence
  Synthetic Construct
aauaccgaggaaugucgggcc

SEQ ID NO: 80 - RNA, 21 nt, Artificial Sequence
  Synthetic Construct
agucgaagacaguucuagggc

SEQ ID NO: 81 - RNA, 21 nt, Artificial Sequence
  Synthetic Construct
uuugcgaaccaacgauagggc

SEQ ID NO: 82 - RNA, 21 nt, Artificial Sequence
  Synthetic Construct
auaggcgaauacacccagcgc

SEQ ID NO: 83 - RNA, 21 nt, Artificial Sequence
  Synthetic Construct
guugucggguuugauguccca

SEQ ID NO: 84 - RNA, 21 nt, Artificial Sequence
  Synthetic Construct
uaccguuccaucugcccgcgc

SEQ ID NO: 85 - RNA, 21 nt, Artificial Sequence
  Synthetic Construct
aaagcgccuccgauaggccgc

SEQ ID NO: 86 - RNA, 21 nt, Artificial Sequence
  Synthetic Construct
gucgaagacaguucuagggcc

SEQ ID NO: 87 - RNA, 21 nt, Artificial Sequence
  Synthetic Construct
ggcgaaguucugguuguccgc

SEQ ID NO: 88 - RNA, 21 nt, Artificial Sequence
  Synthetic Construct
uaggcgaauacacccagcgcc

SEQ ID NO: 89 - RNA, 21 nt, Artificial Sequence
  Synthetic Construct
ugcgaaccaacgauagguggc

SEQ ID NO: 90 - RNA, 21 nt, Artificial Sequence
  Synthetic Construct
ggcgaauacacccagcgccca

SEQ ID NO: 91 - RNA, 21 nt, Artificial Sequence
  Synthetic Construct
gaauccgcguagaagggcggc

SEQ ID NO: 92 - RNA, 21 nt, Artificial Sequence
  Synthetic Construct
ugugucgguggcaccuucgca

SEQ ID NO: 93 - RNA, 21 nt, Artificial Sequence
  Synthetic Construct
ggccgaaagaaagaaaugggc

SEQ ID NO: 94 - RNA, 21 nt, Artificial Sequence
  Synthetic Construct
uacgagacagaacaacggcgc

SEQ ID NO: 95 - RNA, 21 nt, Artificial Sequence
  Synthetic Construct
aacaacggcgaacaggagcgc

SEQ ID NO: 96 - RNA, 21 nt, Artificial Sequence
  Synthetic Construct
ggagcgguugugaacuggcgc

SEQ ID NO: 97 - RNA, 21 nt, Artificial Sequence
  Synthetic Construct
uggagcgguugugaacuggca

SEQ ID NO: 98 - RNA, 21 nt, Artificial Sequence
  Synthetic Construct
auucccggcuacaaggacccu

SEQ ID NO: 99 - RNA, 19 nt, Artificial Sequence
  Synthetic Construct
uucgcguaguugacugugg

SEQ ID NO: 100 - RNA, 21 nt, Artificial Sequence
  Synthetic Construct
uuaccgcuaccugaggucggc

SEQ ID NO: 101 - RNA, 21 nt, Artificial Sequence
  Synthetic Construct
uuagcggcgcaccuucccgca

SEQ ID NO: 102 - RNA, 21 nt, Artificial Sequence
  Synthetic Construct
uucgcacgccucgaaucccgc

SEQ ID NO: 103 - RNA, 21 nt, Artificial Sequence
  Synthetic Construct
agucggaccuccuuaagccgc

SEQ ID NO: 104 - RNA, 21 nt, Artificial Sequence
  Synthetic Construct
aagucgcaaagacguagggcc

SEQ ID NO: 105 - RNA, 21 nt, Artificial Sequence
  Synthetic Construct
agucgucccucugcagucgga

SEQ ID NO: 106 - RNA, 21 nt, Artificial Sequence
  Synthetic Construct
uguccgaacucgucauuggca

SEQ ID NO: 107 - RNA, 21 nt, Artificial Sequence
  Synthetic Construct
uagcgaccgcaccuggaccca

SEQ ID NO: 108 - RNA, 21 nt, Artificial Sequence
  Synthetic Construct
guccgacagugucuccugcgc

SEQ ID NO: 109 - RNA, 21 nt, Artificial Sequence
  Synthetic Construct
guaacggcccagacgugggca

SEQ ID NO: 110 - RNA, 21 nt, Artificial Sequence
  Synthetic Construct
uacguagccaagccggugcgc

SEQ ID NO: 111 - RNA, 21 nt, Artificial Sequence
  Synthetic Construct
uagcgguucagaaucaagcgc

SEQ ID NO: 112 - RNA, 21 nt, Artificial Sequence
  Synthetic Construct
uagucggaccuccuuaagcca

SEQ ID NO: 113 - RNA, 21 nt, Artificial Sequence
  Synthetic Construct
uucguccagugacagcacggc

SEQ ID NO: 114 - RNA, 55 nt, Artificial Sequence
  Synthetic Construct
aaaacucgagugagcgcgggcgcuggguguauucguuacuguaaagccacagaug

SEQ ID NO: 115 - RNA, 55 nt, Artificial Sequence
  Synthetic Construct
aaaacucgagugagcgaccgcccuucuacgcggauuuacuguaaagccacagaug

SEQ ID NO: 116 - RNA, 55 nt, Artificial Sequence
  Synthetic Construct
aaaacucgagugagcgcgcgaaggugccaccgacauaacuguaaagccacagaug

SEQ ID NO: 117 - RNA, 55 nt, Artificial Sequence
  Synthetic Construct
aaaacucgagugagcgacccauuucuuucuuucgguuacuguaaagccacagaug

SEQ ID NO: 118 - RNA, 55 nt, Artificial Sequence
  Synthetic Construct
aaaacucgagugagcgacgccguuguucugucucgugacuguaaagccacagaug

SEQ ID NO: 119 - RNA, 55 nt, Artificial Sequence
  Synthetic Construct
aaaacucgagugagcgacgcuccuguucgccguuguuacuguaaagccacagaug

SEQ ID NO: 120 - RNA, 55 nt, Artificial Sequence
  Synthetic Construct
aaaacucgagugagcgacgccaguucacaaccgcuuuacuguaaagccacagaug

SEQ ID NO: 121 - RNA, 55 nt, Artificial Sequence
  Synthetic Construct
aaaacucgagugagcgcgccaguucacaaccgcuuugacuguaaagccacagaug

SEQ ID NO: 122 - RNA, 55 nt, Artificial Sequence
  Synthetic Construct
aaaacucgagugagcgcggguccuuguagccgggaauacuguaaagccacagaug

SEQ ID NO: 123 - RNA, 55 nt, Artificial Sequence
  Synthetic Construct
uuuuacuaguaggcgugggcgcuggguguauucgccagcaucuguggcuuuacag

SEQ ID NO: 124 - RNA, 55 nt, Artificial Sequence
  Synthetic Construct
uuuuacuaguaggcggccgcccuucuacgcggauucagcaucuguggcuuuacag

SEQ ID NO: 125 - RNA, 55 nt, Artificial Sequence
  Synthetic Construct
uuuuacuaguaggcgugcgaaggugccaccgacacaagcaucuguggcuuuacag

SEQ ID NO: 126 - RNA, 55 nt, Artificial Sequence
  Synthetic Construct
uuuuacuaguaggcggcccauuucuuucuuucggccagcaucuguggcuuuacag

SEQ ID NO: 127 - RNA, 55 nt, Artificial Sequence
  Synthetic Construct
uuuuacuaguaggcggcgccguuguucugucucguaagcaucuguggcuuuacag

SEQ ID NO: 128 - RNA, 55 nt, Artificial Sequence
  Synthetic Construct
uuuuacuaguaggcggcgcuccuguucgccguuguuagcaucuguggcuuuacag

SEQ ID NO: 129 - RNA, 55 nt, Artificial Sequence
  Synthetic Construct
uuuuacuaguaggcggcgccaguucacaaccgcuccagcaucuguggcuuuacag

SEQ ID NO: 130 - RNA, 55 nt, Artificial Sequence
  Synthetic Construct
uuuuacuaguaggcgugccaguucacaaccgcuccaagcaucuguggcuuuacag

SEQ ID NO: 131 - RNA, 55 nt, Artificial Sequence
  Synthetic Construct
uuuuacuaguaggcguggguccuuguagccgggaauagcaucuguggcuuuacag

SEQ ID NO: 132 - RNA, 55 nt, Artificial Sequence
  Synthetic Construct
aaaacucgagugagcgacccacagucaacuacgcgagacuguaaagccacagaug

SEQ ID NO: 133 - RNA, 55 nt, Artificial Sequence
  Synthetic Construct
uuuuacuaguaggcggcccacagucaacuacgcgaaagcaucuguggcuuuacag

SEQ ID NO: 134 - RNA, 55 nt, Artificial Sequence
  Synthetic Construct
aaaacucgagugagcgaccgaccucagguagcgguagacuguaaagccacagaug

SEQ ID NO: 135 - RNA, 55 nt, Artificial Sequence
  Synthetic Construct
uuuuacuaguaggcggccgaccucagguagcgguaaagcaucuguggcuuuacag

SEQ ID NO: 136 - RNA, 55 nt, Artificial Sequence
  Synthetic Construct
aaaacucgagugagcgcgcgggaaggugcgccgcuagacuguaaagccacagaug

SEQ ID NO: 137 - RNA, 55 nt, Artificial Sequence
  Synthetic Construct
uuuuacuaguaggcgugcgggaaggugcgccgcuaaagcaucuguggcuuuacag

SEQ ID NO: 138 - RNA, 55 nt, Artificial Sequence
  Synthetic Construct
aaaacucgagugagcgacgggauucgaggcgugcgagacuguaaagccacagaug

SEQ ID NO: 139 - RNA, 55 nt, Artificial Sequence
  Synthetic Construct
uuuuacuaguaggcggcgggauucgaggcgugcgaaagcaucuguggcuuuacag

SEQ ID NO: 140 - RNA, 55 nt, Artificial Sequence
  Synthetic Construct
aaaacucgagugagcgacggcuuaaggagguccgauuacuguaaagccacagaug

SEQ ID NO: 141 - RNA, 55 nt, Artificial Sequence
  Synthetic Construct
uuuuacuaguaggcggcggcuuaaggagguccgacuagcaucuguggcuuuacag

SEQ ID NO: 142 - RNA, 55 nt, Artificial Sequence
  Synthetic Construct
aaaacucgagugagcgagcccuacgucuuugcgauuuacuguaaagccacagaug

SEQ ID NO: 143 - RNA, 55 nt, Artificial Sequence
  Synthetic Construct
uuuuacuaguaggcgggcccuacgucuuugcgacuuagcaucuguggcuuuacag

SEQ ID NO: 144 - RNA, 55 nt, Artificial Sequence
  Synthetic Construct
aaaacucgagugagcgcccgacugcagagggacgauuacuguaaagccacagaug

SEQ ID NO: 145 - RNA, 55 nt, Artificial Sequence
  Synthetic Construct
uuuuacuaguaggcguccgacugcagagggacgacuagcaucuguggcuuuacag

SEQ ID NO: 146 - RNA, 55 nt, Artificial Sequence
  Synthetic Construct
aaaacucgagugagcgcgccaaugacgaguucggaugacuguaaagccacagaug

SEQ ID NO: 147 - RNA, 55 nt, Artificial Sequence
  Synthetic Construct
uuuuacuaguaggcgugccaaugacgaguucggacaagcaucuguggcuuuacag

SEQ ID NO: 148 - RNA, 55 nt, Artificial Sequence
  Synthetic Construct
aaaacucgagugagcgcggguccaggugcggucguugacuguaaagccacagaug

SEQ ID NO: 149 - RNA, 55 nt, Artificial Sequence
  Synthetic Construct
uuuuacuaguaggcguggguccaggugcggucgcuaagcaucuguggcuuuacag

SEQ ID NO: 150 - RNA, 55 nt, Artificial Sequence
  Synthetic Construct
aaaacucgagugagcgacgcaggagacacugucggauacuguaaagccacagaug

SEQ ID NO: 151 - RNA, 55 nt, Artificial Sequence
  Synthetic Construct
uuuuacuaguaggcggcgcaggagacacugucggacagcaucuguggcuuuacag

SEQ ID NO: 152 - RNA, 55 nt, Artificial Sequence
  Synthetic Construct
aaaacucgagugagcgcgcccacgucugggccguuauacuguaaagccacagaug

SEQ ID NO: 153 - RNA, 55 nt, Artificial Sequence
  Synthetic Construct
uuuuacuaguaggcgugcccacgucugggccguuacagcaucuguggcuuuacag

SEQ ID NO: 154 - RNA, 55 nt, Artificial Sequence
  Synthetic Construct
aaaacucgagugagcgacgcaccggcuuggcuacgugacuguaaagccacagaug

SEQ ID NO: 155 - RNA, 55 nt, Artificial Sequence
  Synthetic Construct
uuuuacuaguaggcggcgcaccggcuuggcuacguaagcaucuguggcuuuacag

SEQ ID NO: 156 - RNA, 55 nt, Artificial Sequence
  Synthetic Construct
aaaacucgagugagcgacgcuugauucugaaccguugacuguaaagccacagaug

SEQ ID NO: 157 - RNA, 55 nt, Artificial Sequence
  Synthetic Construct
uuuuacuaguaggcggcgcuugauucugaaccgcuaagcaucuguggcuuuacag

SEQ ID NO: 158 - RNA, 55 nt, Artificial Sequence
  Synthetic Construct
aaaacucgagugagcgcggcuuaaggagguccgauugacuguaaagccacagaug

SEQ ID NO: 159 - RNA, 55 nt, Artificial Sequence
  Synthetic Construct
uuuuacuaguaggcguggcuuaaggagguccgacuaagcaucuguggcuuuacag

SEQ ID NO: 160 - RNA, 55 nt, Artificial Sequence
  Synthetic Construct
aaaacucgagugagcgaccgugcugucacuggacgagacuguaaagccacagaug

SEQ ID NO: 161 - RNA, 55 nt, Artificial Sequence
  Synthetic Construct
uuuuacuaguaggcggccgugcugucacuggacgaaagcaucuguggcuuuacag

SEQ ID NO: 162 - RNA, 6 nt, Homo sapiens
ggggcc

SEQ ID NO: 163 - RNA, 3356 nt, Homo sapiens
acguaaccuacggugucccgcuaggaaagagaggugcgucaaacagcgacaaguuccgcccacguaaaagaugacgcuuggugugucagccgucccugcugcccgguugcuucucuuuugggggcggggucuagcaagagcagguguggguuuaggagauaucuccggagcauuuggauaaugugacaguuggaaugcagugaugucgacucuuugcccaccgccaucuccagcuguugccaagacagagauugcuuuaaguggcaaaucaccuuuauuagcagcuacuuuugcuuacugggacaauauucuugguccuagaguaaggcacauuugggcuccaaagacagaacagguacuucucagugauggagaaauaacuuuucuugccaaccacacucuaaauggagaaauccuucgaaaugcagagaguggugcuauagauguaaaguuuuuugucuugucugaaaagggagugauuauuguuucauuaaucuuugauggaaacuggaauggggaucgcagcacauauggacuaucaauuauacuuccacagacagaacuuaguuucuaccucccacuucauagaguguguguugauagauuaacacauauaauccggaaaggaagaauauggaugcauaaggaaagacaagaaaauguccagaagauuaucuuagaaggcacagagagaauggaagaucagggucagaguauuauuccaaugcuuacuggagaagugauuccuguaauggaacugcuuucaucuaugaaaucacacaguguuccugaagaaauagauauagcugauacaguacucaaugaugaugauauuggugacagcugucaugaaggcuuucuucucaaugccaucagcucacacuugcaaaccuguggcuguuccguuguaguagguagcagugcagagaaaguaaauaagauagucagaacauuaugccuuuuucugacuccagcagagagaaaaugcuccagguuaugugaagcagaaucaucauuuaaauaugagucagggcucuuuguacaaggccugcuaaaggauucaacuggaagcuuugugcugccuuuccggcaagucauguaugcuccauaucccaccacacacauagauguggaugucaauacugugaagcagaugccacccugucaugaacauauuuauaaucagcguagauacaugagauccgagcugacagccuucuggagagccacuucagaagaagacauggcucaggauacgaucaucuacacugacgaaagcuuuacuccugauuugaauauuuuucaagaugucuuacacagagacacucuagugaaagccuuccuggaucaggucuuucagcugaaaccuggcuuaucucucagaaguacuuuccuugcacaguuucuacuuguccuucacagaaaagccuugacacuaauaaaauauauagaagacgauacgcagaagggaaaaaagcccuuuaaaucucuucggaaccugaagauagaccuugauuuaacagcagagggcgaucuuaacauaauaauggcucuggcugagaaaauuaaaccaggccuacacucuuuuaucuuuggaagaccuuucuacacuagugugcaagaacgagauguucuaaugacuuuuuaaauguguaacuuaauaagccuauuccaucacaaucaugaucgcugguaaaguagcucagugguguggggaaacguuccccuggaucauacuccagaauucugcucucagcaauugcaguuaaguaaguuacacuacaguucucacaagagccugugaggggaugucaggugcaucauuacauugggugucucuuuuccuagauuuaugcuuuugggauacagaccuauguuuacaauauaauaaauauuauugcuaucuuuuaaagauauaauaauaggauguaaacuugaccacaacuacuguuuuuuugaaauacaugauucaugguuuacaugugucaaggugaaaucugaguuggcuuuuacagauaguugacuuucuaucuuuuggcauucuuugguguguagaauuacuguaauacuucugcaaucaacugaaaacuagagccuuuaaaugauuucaauuccacagaaagaaagugagcuugaacauaggaugagcuuuagaaagaaaauugaucaagcagauguuuaauuggaauugauuauuagauccuacuuuguggauuuagucccugggauucagucuguagaaaugucuaauaguucucuauaguccuuguuccuggugaaccacaguuaggguguuuuguuuauuuuauuguucuugcuauuguugauauucuauguaguugagcucuguaaaaggaaauuguauuuuauguuuuaguaauuguugccaacuuuuuaaauuaauuuucauuauuuuugagccaaauugaaaugugcaccuccugugccuuuuuucuccuuagaaaaucuaauuacuuggaacaaguucagauuucacuggucagucauuuucaucuuguuuucuucuugcuaagucuuaccauguaccugcuuuggcaaucauugcaacucugagauuauaaaaugccuuagagaauauacuaacuaauaagaucuuuuuuucagaaacagaaaauaguuccuugaguacuuccuucuugcauuucugccuauguuuuugaaguuguugcuguuugccugcaauaggcuauaaggaauagcaggagaaauuuuacugaagugcuguuuuccuaggugcuacuuuggcagagcuaaguuaucuuuuguuuucuuaaugcguuuggaccauuuugcuggcuauaaaauaacugauuaauauaauucuaacacaauguugacauuguaguuacacaaacacaaauaaauauuuuauuuaaaauucuggaaguaauauaaaagggaaaauauauuuauaagaaagggauaaagguaauagagcccuucugccccccacccaccaaauuuacacaacaaaaugacauguucgaaugugaaaggucauaauagcuuucccaucaugaaucagaaagauguggacagcuugauguuuuagacaaccacugaacuagaugacuguuguacuguagcucagucauuuaaaaaauauauaaauacuaccuuguagugucccauacuguguuuuuuacaugguagauucuuauuuaagugcuaacugguuauuuucuuuggcugguuuauuguacuguuauacagaauguaaguuguacagugaaauaaguuauuaaagcauguguaaacauuguuauauaucuuuucuccuaaauggagaauuuugaauaaaauauauuugaaauuuuaaaaaaaaaaaaaaaaaa

SEQ ID NO: 164 - RNA, 4 nt, Homo sapiens
ccug

SEQ ID NO: 165 - RNA, 3261 nt, Homo sapiens
gggcggggcugcgguugcggugccugcgcccgcggcggcggaggcgcaggcgguggcgaguggauaucuccggagcauuuggauaaugugacaguuggaaugcagugaugucgacucuuugcccaccgccaucuccagcuguugccaagacagagauugcuuuaaguggcaaaucaccuuuauuagcagcuacuuuugcuuacugggacaauauucuugguccuagaguaaggcacauuugggcuccaaagacagaacagguacuucucagugauggagaaauaacuuuucuugccaaccacacucuaaauggagaaauccuucgaaaugcagagaguggugcuauagauguaaaguuuuuugucuugucugaaaagggagugauuauuguuucauuaaucuuugauggaaacuggaauggggaucgcagcacauauggacuaucaauuauacuuccacagacagaacuuaguuucuaccucccacuucauagaguguguguugauagauuaacacauauaauccggaaaggaagaauauggaugcauaaggaaagacaagaaaauguccagaagauuaucuuagaaggcacagagagaauggaagaucagggucagaguauuauuccaaugcuuacuggagaagugauuccuguaauggaacugcuuucaucuaugaaaucacacaguguuccugaagaaauagauauagcugauacaguacucaaugaugaugauauuggugacagcugucaugaaggcuuucuucucaaugccaucagcucacacuugcaaaccuguggcuguuccguuguaguagguagcagugcagagaaaguaaauaagauagucagaacauuaugccuuuuucugacuccagcagagagaaaaugcuccagguuaugugaagcagaaucaucauuuaaauaugagucagggcucuuuguacaaggccugcuaaaggauucaacuggaagcuuugugcugccuuuccggcaagucauguaugcuccauaucccaccacacacauagauguggaugucaauacugugaagcagaugccacccugucaugaacauauuuauaaucagcguagauacaugagauccgagcugacagccuucuggagagccacuucagaagaagacauggcucaggauacgaucaucuacacugacgaaagcuuuacuccugauuugaauauuuuucaagaugucuuacacagagacacucuagugaaagccuuccuggaucaggucuuucagcugaaaccuggcuuaucucucagaaguacuuuccuugcacaguuucuacuuguccuucacagaaaagccuugacacuaauaaaauauauagaagacgauacgcagaagggaaaaaagcccuuuaaaucucuucggaaccugaagauagaccuugauuuaacagcagagggcgaucuuaacauaauaauggcucuggcugagaaaauuaaaccaggccuacacucuuuuaucuuuggaagaccuuucuacacuagugugcaagaacgagauguucuaaugacuuuuuaaauguguaacuuaauaagccuauuccaucacaaucaugaucgcugguaaaguagcucagugguguggggaaacguuccccuggaucauacuccagaauucugcucucagcaauugcaguuaaguaaguuacacuacaguucucacaagagccugugaggggaugucaggugcaucauuacauugggugucucuuuuccuagauuuaugcuuuugggauacagaccuauguuuacaauauaauaaauauuauugcuaucuuuuaaagauauaauaauaggauguaaacuugaccacaacuacuguuuuuuugaaauacaugauucaugguuuacaugugucaaggugaaaucugaguuggcuuuuacagauaguugacuuucuaucuuuuggcauucuuugguguguagaauuacuguaauacuucugcaaucaacugaaaacuagagccuuuaaaugauuucaauuccacagaaagaaagugagcuugaacauaggaugagcuuuagaaagaaaauugaucaagcagauguuuaauuggaauugauuauuagauccuacuuuguggauuuagucccugggauucagucuguagaaaugucuaauaguucucuauaguccuuguuccuggugaaccacaguuaggguguuuuguuuauuuuauuguucuugcuauuguugauauucuauguaguugagcucuguaaaaggaaauuguauuuuauguuuuaguaauuguugccaacuuuuuaaauuaauuuucauuauuuuugagccaaauugaaaugugcaccuccugugccuuuuuucuccuuagaaaaucuaauuacuuggaacaaguucagauuucacuggucagucauuuucaucuuguuuucuucuugcuaagucuuaccauguaccugcuuuggcaaucauugcaacucugagauuauaaaaugccuuagagaauauacuaacuaauaagaucuuuuuuucagaaacagaaaauaguuccuugaguacuuccuucuugcauuucugccuauguuuuugaaguuguugcuguuugccugcaauaggcuauaaggaauagcaggagaaauuuuacugaagugcuguuuuccuaggugcuacuuuggcagagcuaaguuaucuuuuguuuucuuaaugcguuuggaccauuuugcuggcuauaaaauaacugauuaauauaauucuaacacaauguugacauuguaguuacacaaacacaaauaaauauuuuauuuaaaauucuggaaguaauauaaaagggaaaauauauuuauaagaaagggauaaagguaauagagcccuucugccccccacccaccaaauuuacacaacaaaaugacauguucgaaugugaaaggucauaauagcuuucccaucaugaaucagaaagauguggacagcuugauguuuuagacaaccacugaacuagaugacuguuguacuguagcucagucauuuaaaaaauauauaaauacuaccuuguagugucccauacuguguuuuuuacaugguagauucuuauuuaagugcuaacugguuauuuucuuuggcugguuuauuguacuguuauacagaauguaaguuguacagugaaauaaguuauuaaagcauguguaaacauuguuauauaucuuuucuccuaaauggagaauuuugaauaaaauauauuugaaauuuuaaaaaaaaaaaaaaaaaa

SEQ ID NO: 166 - RNA, 1957 nt, Homo sapiens
acguaaccuacggugucccgcuaggaaagagaggugcgucaaacagcgacaaguuccgcccacguaaaagaugacgcuugauaucuccggagcauuuggauaaugugacaguuggaaugcagugaugucgacucuuugcccaccgccaucuccagcuguugccaagacagagauugcuuuaaguggcaaaucaccuuuauuagcagcuacuuuugcuuacugggacaauauucuugguccuagaguaaggcacauuugggcuccaaagacagaacagguacuucucagugauggagaaauaacuuuucuugccaaccacacucuaaauggagaaauccuucgaaaugcagagaguggugcuauagauguaaaguuuuuugucuugucugaaaagggagugauuauuguuucauuaaucuuugauggaaacuggaauggggaucgcagcacauauggacuaucaauuauacuuccacagacagaacuuaguuucuaccucccacuucauagaguguguguugauagauuaacacauauaauccggaaaggaagaauauggaugcauaaggaaagacaagaaaauguccagaagauuaucuuagaaggcacagagagaauggaagaucagggucagaguauuauuccaaugcuuacuggagaagugauuccuguaauggaacugcuuucaucuaugaaaucacacaguguuccugaagaaauagauauagcugauacaguacucaaugaugaugauauuggugacagcugucaugaaggcuuucuucucaaguaagaauuuuucuuuucauaaaagcuggaugaagcagauaccaucuuaugcucaccuaugacaagauuuggaagaaagaaaauaacagacugucuacuuagauuguucuagggacauuacguauuugaacuguugcuuaaauuuguguuauuuuucacucauuauauuucuauauauauuugguguuauuccauuugcuauuuaaagaaaccgaguuuccaucccagacaagaaaucauggccccuugcuugauucugguuucuuguuuuacuucucauuaaagcuaacagaauccuuucauauuaaguuguacuguagaugaacuuaaguuauuuaggcguagaacaaaauuauucauauuuauacugaucuuuuuccauccagcaguggaguuuaguacuuaagaguuugugcccuuaaaccagacucccuggauuaaugcuguguacccgugggcaaggugccugaauucucuauacaccuauuuccucaucuguaaaauggcaauaauaguaauaguaccuaauguguaggguuguuauaagcauugaguaagauaaauaauauaaagcacuuagaacagugccuggaacauaaaaacacuuaauaauagcucauagcuaacauuuccuauuuacauuucuucuagaaauagccaguauuuguugagugccuacauguuaguuccuuuacuaguugcuuuacauguauuaucuuauauucuguuuuaaaguuucuucacaguuacagauuuucaugaaauuuuacuuuuaauaaaagagaaguaaaaguauaaaguauucacuuuuauguucacagucuuuuccuuuaggcucaugauggaguaucagaggcaugaguguguuuaaccuaagagccuuaauggcuugaaucagaagcacuuuaguccuguaucuguucagugucagccuuucauacaucauuuuaaaucccauuugacuuuaaguaagucacuuaaucucucuacaugucaauuucuucagcuauaaaaugaugguauuucaauaaauaaauacauuaauuaaaugauauuauacugacuaauugggcuguuuuaaggcucaauaagaaaauuucugugaaaggucucuagaaaauguagguuccuauacaaauaaaagauaacauugugcuuauaaaaaaaa